(19) United States
(12) Patent Application Publication
(10) Pub. No.: US 2005/0171131 A1
(43) Pub. Date: Aug. 4, 2005

(54) Diaminopyrimidine derivatives as growth hormone secrectgogue receptor (GHS-R) antagonists

(76) Inventors: Christi Kosogof, Crystal Lake, IL (US); Bo Liu, Waukegan, IL (US); Gang Liu, Gurnee, IL (US); Mei Liu, Gurnee, IL (US); Lissa T. J. Nelson, Highland Park, IL (US); Michael D. Serby, Mundelein, IL (US); Hing L. Sham, Vernon Hills, IL (US); Bruce G. Szczepankiewicz, Lake Villa, IL (US); Zhili Xin, Lake Bluff, IL (US); Hongyu Zhao, Buffalo Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/947,823

(22) Filed: Sep. 23, 2004

Related U.S. Application Data
(60) Provisional application No. 60/506,663, filed on Sep. 26, 2003.

(51) Int. Cl. A61K 31/506; C07D 43/02

(52) U.S. Cl. 514/275; 544/329

(57) ABSTRACT

The present invention is related to compounds of formula (I), 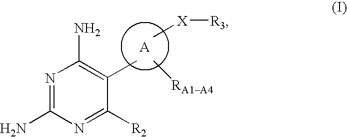 or a therapeutically suitable salt or prodrug thereof, the preparation of the compounds, compositions containing the compounds and the use of the compounds in the prevention or treatment of disorders regulated by the action of ghrelin receptor, including Prader-Willi syndrome, eating disorder, weight gain, weight-loss maintainance following diet and exercise, obesity, and disorders associated with obesity such as noninsulin dependent diabetes mellitus.

This application claims priority from U.S. Provisional Patent Application Ser. No. 60/506,663, filed Sep. 26, 2003, which is incorporated herein by reference.

### TECHNICAL FIELD

The present invention is directed to compounds that are antagonists of the growth hormone secrectgogue receptor (GHS-R), the preparation of the compounds, compositions containing the compounds and the use of the compounds in the prevention or treatment of disorders regulated by the activation of GHSR, including Prader-Willi syndrome, eating disorder, weight gain, weight-loss maintainance following diet and exercise, obesity, and disorders associated with obesity such as non-insulin dependent diabetes mellitus.

### BACKGROUND OF THE INVENTION

Obesity is a common and very serious public health problem as it increases a person's risk for a number of serious conditions, including diabetes, heart disease, stroke, high blood pressure, and some types of cancers. Considerable increase in the number of obese individuals over the past two decades has created profound public health implications. Although studies have demonstrated that reduction in obesity by diet and exercise reduces the associated risk factors dramatically, these treatments are largely unsuccessful considering obesity is strongly associated with genetically inherited factors that contribute to increased appetite, preferences for highly caloric foods, reduced physical activity, and increased lipogenic metabolism.

Growth hormone (GH) is not only of importance for linear body growth but is also of major importance for the maintenance of body composition, metabolism and heart function in adult life. GH release from the anterior pituitary is regulated by the stimulatory peptide GH-releasing hormone (GHRH) and the inhibitory peptide somatostatin, Frohman, L., Jansson, J.-O., Endocr. Rev. (1986) 7:223-253. Early research identified small GHreleasing peptides (GHRPs) derived from the pentapeptide met-enkephalin, Momany, F., et. al., Endocrinology (1981) 108:31-39. Further efforts led to the development of a number of peptidyl and non-peptidyl growth hormone secrectgogues (GHSs), including the orally-active, non-peptidyl GH secretagogue MK677, Svensson, J., et. al., J. Clin. Endocrinol. Metab. (1998) 83:362-369. Later efforts cloned a seven-transmembrane G-protein coupled receptor (GPCR) that was a target for the GHSs, Howard, A., et. al., Science (1996) 273:974-977.

This GHS-receptor (GHS-R) is localized in the hypothalamus and in the pituitary, but also in other brain areas such as the hippocampus as well as the pancreas. Recently, an endogenous ligand for the GHS-R, ghrelin, an acylated peptide consisting of 28 amino acids was isolated, Kojima, M., et. al., Nature (1999) 402:656-660. Since then, ghrelin has been found to be localized in the hypothalamic-pituitary area where it stimulates the release of GH to the circulation, but is also found in the highest concentration in the stomach.

Biological evidence indicates that ghrelin has an important role in the regulation of metabolism and energy expenditure. Ghrelin was found to stimulate food intake and weight gain when administered either systemically or intraventricularly in rodents, Nakazato M, et. al., Nature 2001;409:194-198; Asakawa A, et. al., Gastroenterology (2001) 120:337-345. Ghrelin was also found to be more potent than any other orexigenic peptide except neuropeptide Y (NPY). The orexigenic activity of centrally administered ghrelin is thought to be mediated by brain NPY and AGRP, two neuropeptides with potent orexigenic actions, Kamegai, J., et. al., Endocrinology (2000) 141:4797-4800. It was also recognized that the appetite activity of centrally administered ghrelin can be blocked by co-administration of a NPY-Y1 receptor antagonist. In addition, ghrelin was found to reverse leptin-induced inhibition of food intake, Shintani, M., et. al., Diabetes (2001) 50:227-232. Ghrelin exerts its actions, in the arcuate nucleus and paraventricular nucleus to influence the interplay of NPY, AGRP and a-MSH circuits. Ghrelin may also act via afferent vagal pathways that terminate in the hypothalamus. In obese patients, the increase in the plasma ghrelin level with diet-induced weight loss is consistent with the hypothesis that ghrelin has a role in the long-term regulation of body weight. Gastric bypass in obese patients is associated with markedly suppressed ghrelin levels, possibly contributing to the weight-reducing effect of the procedure, Cummings, D. E., et. al., N Engl J Med (2002) 346:1623-30.

Intracerebroventricular treatment with the anti-ghrelin antiserum against the N-terminal region twice a day for 5 days in rats decreased significantly both daily food intake and body weight, Murakami, N., et. al., Journal of Endocrinology (2002) 174, 283-288. Transgenic (Tg) rats expressing an antisense ghrelin receptor mRNA under the control of the promoter for tyrosine hydroxylase (TH) selectively attenuated ghrelin receptor protein expression in the arcuate nucleus (Arc). Tg rats had lower body weight and less adipose tissue than did control rats. Daily food intake was reduced, and the stimulatory effect of GHS treatment on feeding was abolished in Tg rats,. Shuto, Y., et. al., J. Clin. Invest. (2002) 109:1429-1436. More recently, a peptide-based GHS-R antagonist, [D-Lys-3]-GHRP, was found to decrease energy intake in lean mice, in mice with diet induced obesity, and in ob/ob obese mice. It also reduced the rate of gastric emptying. Repeated aministration of this GHS-R antagonist decreased body weight and improved glycemic control in ob/ob mice, Asakawa, A. et. al., Gut, (2003), 52:947-952. These data suggest that GHS-R antagonists may be beneficial in the treatment of Prader-Willi syndrome, eating disorder, weight gain, weight-loss maintainance following diet and exercise, obesity, and disorders associated with obesity such as non-insulin dependent diabetes mellitus.

Diaminopyrimidines have been investigated as dihydrofolate reductase (DHFR) inhibitors for the indication of anti-malaria, anti-imflammation and anti-neoplasty. Recent publications indicate that compounds possessing dihydrofolate reductase inhibitory properties may be useful as medicaments when coadministering folate supplements such as folic acid and/or vitamin B-12; Hanauske A. R., Chen, V., Paoletti, P., Niyikiza, C. Oncologist: 2001;6(4):363-73. Morgan, S. L., Baggott, J. E., Vaughn, W. H., Young, P. K., Austin, J. V., Krumdieck, C. L., Alarcon, G. S. Arthritis Rheum: 1990 January;33(1):9-18. Alati, T., Worzalla, J. F., Shih, C., Bewley, J. R., Lewis, S., Moran, R. G., Grindey, G. B. Cancer Res: 1996 May 15;56(10):2331-5. Therefore, compounds of the present invention which antagonize the action of ghrelin, may inhibit the function of DHFR, and may be suitable for drug development as anti-obesity therapeutical agents when administered in conjunction with folate supplements such as folic acid and/or vitamin B-12.

### SUMMARY OF THE INVENTION

The principle embodiment of the present invention is directed to a compound of formula (I), 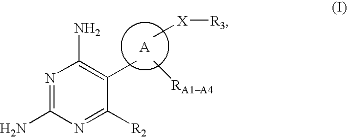
or a therapeutically suitable salt or prodrug thereof, wherein
A is a member selected from the group consisting of aryl, heteroaryl, and heterocycle;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl;
R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;

R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle;
R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;

R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl;
R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle;
X is a member selected from the group consisting of —O—, —NRN—, —CH₂NH—; and
R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

According to another embodiment, the present invention is directed to a pharmaceutical composition comprising a therapeutically effective amount of a compound of formula (I) and a pharmaceutically suitable carrier.

According to another embodiment, the present invention is directed to a method of treating a disorder regulated by GHS-Rs in a mammal, comprising administring of a compound of formula (I).

According to another embodiment, the present invention is directed to a method of treating disorders regulated by the activation of GHS-R, including Prader-Willi syndrome, eating disorder, weight gain, weight-loss maintainance following diet and exercise, obesity, and disorders associated with obesity such as non-insulin dependent diabetes mellitus in a mammal comprising administrating a compound of formula (I).

According to another embodiment, the present invention is directed to a pharmaceutical composition comprising a therapeutically effective amount of a compound of formula (I) coadminstered with an effective amount of a folate supplement.

### DETAILED DESCRIPTION OF THE INVENTION

The principle embodiment of the present invention is directed to a compound of formula (I), 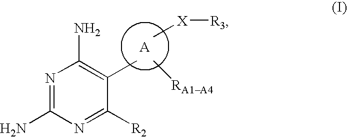
or a therapeutically suitable salt or prodrug thereof, wherein A is a member selected from the group consisting of aryl, heteroaryl, and heterocycle; R₂ is a member selected from the group consisting of alkenyl, alkenyloxyalkyl, alkoxy, alkoxyalkoxy, alkoxyalkoxyalkyl, alkoxyalkyl, o0 alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl; R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and RD together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl,, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl; R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle; X is a member selected from the group consisting of —O—, —NR_{N}—, —CH₂NH—; and R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

In another embodiment of the present invention there is disclosed a compound of formula (I), A is a member selected from the group consisting of aryl, heteroaryl, and heterocycle; X is —NR_{N}—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl; R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{A1}, R_{A2}, R_{A3}, and R_{A4} ae each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl; R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle; and R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

In another embodiment of the present invention there is disclosed a compound of formula (I), A is a member selected from the group consisting of aryl, heteroaryl, and heterocycle; X is —NR_{N}—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl; R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, and (R_{E}R_{F}N)alkyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, and arylalkyl; R_{G} and R_{H} are each independently a member selected from the group consisting of hydrogen, alkoxycarbonyl,-alkyl, alkylcarbonyl, arylalkyl, and arylcarbonyl; R_{J} and R_{K} are each independently a member selected from the group consisting of hydrogen, alkoxycarbonyl, alkyl, and alkylcarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl; R_{L} and R_{M} are each independently a member selected from the group consisting of hydrogen, alkoxycarbonyl, alkyl, and alkylcarbonyl.

In another embodiment of the present invention there is dislosed a compound of formula (I), wherein A is a member selected from the group consisting of aryl, heteroaryl, and heterocycle; X is —NR_{N}—; R₂ is a member selected from the group consisting of alkenyl, alkenyloxyalkyl, alkoxyalkoxyalkyi; alkoxyalkyl, alkyl, arylalkoxyalkyl, arylalkyl, arylalkylalkenyl, arylallcylthioalkyl, aryloxyalkyl, arylthio, arylthioalkyl, carboxy, carboxyalkyl, cyanoaLkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl; R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (R_{G}R_{H}N)alkyl; R_{C} and R_{D} are each independently a member selected from the group consisting of hydrogen, alkyl, aryl, arylalkyl, cycloalkylalkoxyalkyl, heteroarylalkyl, heterocyclealkyl, heterocyclealkoxyalkyl, and (R_{E}R_{F}N)alkyl; R_{E} and R_{F} are each independently a member selected from the group consisting of hydrogen, alkoxycarbonyl; R_{G} and R_{H} are each independently a member selected from the group consisting of hydrogen, and alkoxycarbonyl; R_{A1}, R_{A2}, R_{A3}, and R_{A4} are each independently a member selected from the group consisting of hydrogen, alkyl, aryl, and cycloalkyl.

In another embodiment of the present invention there is disclosed a compound of formula (I), wherein A is a member selected from the group consisting of aryl, heteroaryl, and heterocycle; X is —CH₂NH—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl; R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl; R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle; X is a member selected from the group consisting of —O—, —NRN—, —CH₂NH—; and R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

In another embodiment of the present invention there is disclosed a compound of formula (I), wherein A is a member selected from the group consisting of aryl, heteroaryl, and heterocycle; X is —CH₂NH—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl; R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, and (R_{E}R_{F}N)alkyl, or R_{C} and R_{D} together with the nitrogen atom to which they are attached form a heterocycle; R_{E} and R_{F} are each independently a member selected from the group consisting of hydrogen, alkoxycarbonyl, alkyl, and alkylcarbonyl; R_{G} and R_{H} are each independently a member selected from the group consisting of hydrogen, alkoxycarbonyl, alkyl, alkylcarbonyl, arylalkyl, and arylcarbonyl; R_{J} and R_{K} are each independently a member selected from the group consisting of hydrogen, alkoxycarbonyl, alkyl, and alkylcarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl; R_{L} and R_{M} are each independently a member selected from the group consisting of hydrogen, alkoxycarbonyl, alkyl, and alkylcarbonyl.

In another embodiment of the present invention there is disclosed a compound of formula (I), wherein A is a member selected from the group consisting of aryl, heteroaryl, and heterocycle; X is —CH₂NH—; R₂ is a member selected from the group consisting of alkenyl, alkenyloxyalkyl, alkoxyalkoxyalkyl, alkoxyalkyl, alkyl, arylalkoxyalkyl, arylalkyl, arylalkylalkenyl, arylalkylthioalkyl, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl; R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (R_{G}R_{H}N)alkyl; R_{C} and R_{D} are each independently a member selected from the group consisting of hydrogen, aryl, arylalkyl, cycloalkylalkoxyalkyl, heteroarylalkyl, heterocyclealkyl, heterocyclealkoxyalkyl, and (R_{E}R_{F}N)alkyl; R_{E} and R_{F} are each independently a member selected from the group consisting of hydrogen, and alkoxycarbonyl; R_{G} and R_{H} are each independently a member selected from the group consisting of hydrogen, and alkoxycarbonyl; R_{A1}, R_{A2}, R_{A3}, and RA₄ are each independently a member selected from the group consisting of hydrogen, alkyl, aryl, and cycloalkyl.

In another embodiment of the present invention there is disclosed a compound offormula (I), wherein A is a member selected from the group consisting of aryl, heteroaryl, and heterocycle; X is —O—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl; R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl; R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle; X is a member selected from the group consisting of —O—, —NR_{N}—, —CH₂NH—; and R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

In another embodiment of the present invention there is disclosed a compound of formula (I), wherein A is a member selected from the group consisting of aryl, heteroaryl, and heterocycle; X is —O—; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl; R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, and arylalkyl; R_{G} and R_{H} are each independently a member selected from the group consisting of hydrogen, alkoxycarbonyl, alkyl, alkylcarbonyl, arylalkyl, and arylcarbonyl; R_{J} and R_{K} are each independently a member selected from the group consisting of hydrogen, alkoxycarbonyl, alkyl, and alkylcarbonyl; R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl; R_{L} and R_{M} are each independently a member selected from the group consisting of hydrogen, alkoxycarbonyl, alkyl, and alkylcarbonyl.

In another embodiment of the present invention there is disclosed a compound of formula (I), wherein A is a member selected from the group consisting of aryl, heteroaryl, and heterocycle; X is —O—; R₂ is a member selected from the group consisting of alkenyl, alkenyloxyalkyl, alkoxyalkoxyalkyl, alkoxyalkyl, alkyl, arylalkoxyalkyl, arylalkyl, arylalkylalkenyl, arylalkylthioalkyl, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl; R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (RcR_{H}N)alkyl; R_{C} and R_{D} are each independently a member selected from the group consisting of hydrogen, alkyl, aryl, arylalkyl, cycloalkylalkoxyalkyl, heteroarylalkyl, heterocyclealkyl, heterocyclealkoxyalkyl, and (R_{E}R_{F}N)alkyl; R_{E} and R_{F} are each independently a member selected from the group consisting of hydrogen, and alkoxycarbonyl; R_{G} and R_{H} are each independently a member selected from the group consisting of hydrogen, and alkoxycarbonyl; R_{A1}, R_{A2}, R_{A3}, and R_{A4} are each independently a member selected from the group consisting of hydrogen, alkyl, aryl, and cycloalkyl.

According to another embodiment, the present invention is directed to a pharmaceutical composition comprising a therapeutically effective amount of a compound of formula (I) and a pharmaceutically suitable carrier.

According to another embodiment, the present invention is directed to amethod of treating a disorder regulated by GHS-Rs in a mammal, comprising administring of a compound of formula (I).

According to another embodiment, the present invention is directed to a method of treating disorders regulated by the activation of GHS-R, including Prader-Willi syndrome, eating disorder, weight gain, weight-loss maintainance following diet and exercise, obesity, and disorders associated with obesity such as non-insulin dependent diabetes mellitus in a mammal comprising administrating a compound of formula (I).

According to another embodiment, the present invention is directed to a pharmaceutical composition comprising a therapeutically effective amount of a compound of formula (I) coadininstered with an effective amount of a folate supplement.

### DEFINITIONS

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenyloxy” as used herein, means an alkenyl group, as defined herein, appended to the parent molecular moiety through an oxygen atom.

The term “alkenyloxyalkyl” as used herein, means an alkenyloxy group, as defined herein, appended to the parent molecular moiety through an alkyl group, as defined herein.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, n-butoxy, tert-butoxy, pentyloxy, and hexyloxy.

The term “alkoxyalkoxy” as used herein, means an alkoxy group, as defined herein, appended to the parent molecular moiety through an alkoxy group. Representative example of alkoxyalkoxy include, but are not limited to, 2-(methoxy)ethoxy, 2-(ethoxy)ethoxy, 3-(methoxy)propoxy, and 2-(n-butoxy)ethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, 2-(methoxy)ethoxymethyl, 2-(ethoxy)ethoxymethyl, 3-(methoxy)propoxymethyl, 2-(n-butoxy)ethoxymethyl, and 2-(tert-butoxy)ethoxymethyl.

The term “alkoxyalkyl” as used herein, means an alkoxy group, as defined herein, appended to the parent molecular moiety through an alkyl group, as defined herein. Representative examples of alkoxyalkyl include, but are not limited to, n-butoxymethyl, tert-butoxymethyl, 2-(ethoxy)ethyl, 2-methoxyethyl, and methoxymethyl.

The term “alkoxyalkylcarbonyl” as used herein, means an alkoxyalkyl group, as defined herein, appended to the parent molecular moiety through a carbonyl group, as defined herein. Representative examples of alkoxyalkylcarbonyl include, but are not limited to, n-butoxymethylcarbonyl, tert-butoxymethylcarbonyl, 2-(ethoxy)ethylcarbonyl, 2-methoxyethylcarbonyl, and methoxymethylcarbonyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through a alkyl group, as defined herein.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl, and tert-butoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined 1S herein, appended to the parent molecular moiety through an alkyl group, as defined herein. Representative examples of alkylcarbonylalkyl include, but are not limited to, 2-oxopropyl, 3-oxobutyl, 3-oxopentyl, and 4-oxopentyl.

The term “alkylcarbonyloxy” as used herein, means an alkylcarbonyl group, as defined herein, appended to the parent molecular moiety through an oxygen atom, as defined herein. Representative examples of alkylcarbonyloxy include, but are not limited to, acetyloxy, propionyloxy, 3-oxobutyl, and butyryloxy.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio and ethylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and ethylthiomethyl.

The term “alkylthioalkylcarbonyl” as used herein, means an alkylthioalkyl group, as defined herein, appended to the parent molecular moiety through a carbonyl group, as defined herein. Representative examples of alkylthioalkylcarbonyl include, but are not limited to, methylthiomethylcarbonyl and ethylthiomethylcarbonyl.

The term “alkylthiocarbonyl” as used herein, means an alkylthio group, as defined herein, appended to the parent molecular moiety through a carbonyl group, as defined herein. Representative examples of alkylthiocarbonyl include, but are not limited to, methylthiocarbonyl and ethylthiocarbonyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkynyloxy” as used herein, means an alkynyl group, as defined herein, appended to the parent molecular moiety through an oxygen atom. Representative examples of alkynyloxy include, but are not limited, to but-3-ynyloxy and hex-4-ynyloxy.

The term “alkynyloxyalkyl” as used herein, means an alkynyloxy group, as defined herein, appended to the parent molecular moiety through an alkyl group, as defined herein. Representative examples of alkynyloxyalkyl include, but are not limited, to but-3-ynyloxymethyl and hex-4-ynyloxymethyl.

The term “aryl” as used herein, means a phenyl group, or a bicyclic or a tricyclic fused ring system wherein one or more of the fused rings is a phenyl group. Bicyclic fused ring systems are exemplified by a phenyl group appended to the parent molecular moiety, which is fused to a cycloalkyl group, as defined herein, a phenyl group, a heteroaryl, as defined herein, or a heterocycle as defined herein. Tricyclic fused ring systems are exemplified by an aryl bicyclic fused ring system fused to a cycloalkyl group, as defined herein, a phenyl group, a heteroaryl, as defined herein, or a heterocycle as defined herein. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 0, 1, 2, 3, 4, or 5 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl, hydroxyhaloalkyl, mercapto, nitro, —NZ₅Z₆ and (NZ₅Z₆)alkyl. Representative examples include, but are not limited to, 2-bromophenyl, 2-chlorophenyl, 3-chlorophenyl, 4-chlorophenyl, 3-cyanophenyl, 4-cyanophenyl, 2,3-dichlorophenyl, 3,4-dichlorophenyl, 2,5-dichlorophenyl, 2,4-dimethylphenyl, 3,5-dimethylphenyl, 2-fluoro-3-methylphenyl, 2-fluorophenyl, 3-fluorophenyl, 4-fluorophenyl, 2-methoxyphenyl, 3-methoxyphenyl, 4-methoxyphenyl, 2-methylphenyl, 3-methylphenyl, 4-(methylthio)phenyl, 4-nitrophenyl, 4-(trifluoromethoxy)phenyl, and 3-(trifluoromethyl)phenyl.

The term “arylalkenyl,” as used herein, means an aryl group, as defined herein, appended to the parent molecular moiety through an alkenyl group, as defined herein.

The term “arylalkenyloxy,” as used herein, means an arylalkenyl group, as defined herein, appended to the parent molecular moiety through an oxy group, as defined herein.

The term “arylalkenyloxyalkyl,” as used herein, means an arylalkenyloxy group, as defined herein, appended to the parent molecular moiety through an alkyl group, as defined herein.

The term “arylalkoxy” as used herein, means an aryl group, as defined herein, appended to the parent molecular moiety through an alkoxy group, as defined herein. Representative examples of arylalkoxy include, but are not limited to, benzyloxy, 2-bromobenzyloxy, 2-chlorobenzyloxy, 3-chlorobenzyloxy, 4-chlorobenzyloxy, 2-(4-chlorophenyl)ethoxy, 3-cyanobenzyloxy, 4-cyanobenzyloxy, 2,3-dichlorobenzyloxy, 2,5-dichlorobenzyloxy, 2,4-dimethylbenzyloxy, 3,5-dimethylbenzyloxy, 2-fluoro-3-methylbenzyloxy, 2-fluorobenzyloxy, 4-fluorobenzyloxy, 2-methoxybenzyloxy, 3-methoxybenzyloxy, 4-methoxybenzyloxy, 2-methylbenzyloxy, 3-methylbenzyloxy, 4-(methylthio)benzyloxy, 4-nitrobenzyloxy, 4-(trifluoromethoxy)benzyloxy, and 3-(trifluoromethyl)benzyloxy.

The term “arylalkoxyalkyl” as used herein, means an arylalkoxy group, as defined herein, appended to the parent molecular moiety through an alkyl group, as defined herein. Representative examples of arylalkoxyalkyl include, but are not limited to, benzyloxymethyl, 2-bromobenzyloxymethyl, 2-chlorobenzyloxymethyl, 3-chlorobenzyloxymethyl, 4-chlorobenzyloxymethyl, 4-cyanobenzyloxymethyl, 2,3-dichlorobenzyloxymethyl, 2,5-dichlorobenzyloxymethyl, 2,4-dimethylbenzyloxymethyl, 3,5-dimethylbenzyloxymethyl, 2-fluoro-3-methylbenzyloxymethyl, 2-fluorobenzyloxymethyl, 4-fluorobenzyloxymethyl, 2-methoxybenzyloxymethyl, 3-methoxybenzyloxymethyl, 4-methoxybenzyloxymethyl, 2-methylbenzyloxymethyl, 3-methylbenzyloxymethyl, 4-(methylthio)benzyloxymethyl, 4-nitrobenzyloxymethyl, 4-(trifluoromethoxy)benzyloxymethyl, and 3-(trifluoromethyl)benzyloxymethyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 1-phenylethyl, 3-phenylpropyl, 4-phenylbutyl, 2-naphth-2-ylethyl, 2-bromobenzyl, 4-cyanobenzyl, 1-(4-cyanophenyl)ethyl, 2-chlorobenzyl, 3-chlorobenzyl, 4-chlorobenzyl, 1-(4-chlorophenyl)ethyl, 2-(4-chlorophenyl)ethyl, 2,3-dichlorobenzyl, 2,5-dichlorobenzyl, 2,4-dimethylbenzyl, 3,5-dimethylbenzyl, 2-fluoro-3-methylbenzyl, 2-fluorobenzyl, 4-fluorobenzyl, 2-methoxybenzyl, 3-methoxybenzyl, 4-methoxybenzyl, 2-methylbenzyl, 3-methylbenzyl, 44methylthio)benzyl, 4-nitrobenzyl, 1-(4-nitrophenyl)ethyl, 2-(4-chlorophenyl)ethyl, 4-(trifluoromethoxy)benzyl, and 3-(trifluoromethyl)benzyl.

The term “arylalkylalkenyl,” as used herein, means an arylalkyl group, as defined herein, appended to the parent molecular moiety through an alkenyl group, as defined herein.

The term “arylalkylthio” as used herein, means an arylalkyl group, as defined herein, appended to the parent molecular moiety through a sulfur atom. Representative examples of arylalkylthio include, but are not limited to, benzylthio, 2-phenylethylthio, 1-phenylethylthio, 3-phenylpropylthio, 4-phenylbutylthio, 2-naphth-2-ylethylthio, 2-bromobenzylthio, 4-cyanobenzylthio, 1-(4-cyanophenyl)ethyl, 2-chlorobenzylthio, 3-chlorobenzylthio, 4-chlorobenzylthio, 1-(4-chlorophenyl)ethylthio, 2-(4-chlorophenyl)ethylthio, 2,3-dichlorobenzylthio, 2,5-dichlorobenzylthio, 2,4-dimethylbenzylthio, 3,5-dimethylbenzylthio, 2-fluoro-3-methylbenzylthio, 2-fluorobenzylthio, 4-fluorobenzylthio, 2-methoxybenzylthio, 3-methoxybenzylthio, 4-methoxybenzylthio, 2-methylbenzylthio, 3-methylbenzylthio, 4-(methylthio)benzylthio, 4-nitrobenzylthio, 1-(4-nitrophenyl)ethylthio, 2-(4-chlorophenyl)ethylthio, 4-(trifluoromethoxy)benzylthio, and 3-(trifluoromethyl)benzylthio.

The term “arylalkylthioalkyl” as used herein, means an arylalkylthio group, as defined herein, appended to the parent molecular moiety through an alkyl group, as defined herein. Representative examples of arylalkylthio include, but are not limited to, benzylthiomethyl, 2-phenylethylthiomethyl, 1-phenylethylthiomethyl, 3-phenylpropylthiomethyl, 4-phenylbutylthiomethyl, 2-naphth-2-ylethylthiomethyl, 2-bromobenzylthiomethyl, 4-cyanobenzylthiomethyl, 1-(4-cyanophenyl)ethylmethyl, 2-chlorobenzylthiomethyl, 3-chlorobenzylthiomethyl, 4-chlorobenzylthiomethyl, 1-(4-chlorophenyl)ethylthiomethyl, 2-(4-chlorophenyl)ethylthiomethyl, 2,3-dichlorobenzylthiomethyl, 2,5-dichlorobenzylthiomethyl, 2,4-dimethylbenzylthiomethyl, 3,5-dimethylbenzylthiomethyl, 2-fluoro-3-methylbenzylthiomethyl, 2-fluorobenzylthiomethyl, 4-fluorobenzylthiomethyl, 2-methoxybenzylthiomethyl, 3-methoxybenzylthiomethyl, 4-methoxybenzylthiomethyl, 2-methylbenzylthiomethyl, 3-methylbenzylthiomethyl, 4-(methylthio)benzylthiomethyl, 4-nitrobenzylthiomethyl, 1-(4-nitrophenyl)ethylthiomethyl, 2-(4-chlorophenyl)ethylthiomethyl, 4-(trifluoromethoxy)benzylthiomethyl, and 3-(trifluoromethyl)benzylthiomethyl.

The term “arylcarbonyl” as used herein, means an aryl group, as defined herein, appended to the parent molecular moiety through a carbonyl group, as defined herein. Representative examples of arylcarbonyl include, but are not limited to, benzoyl, naphthoyl, 2-bromo benzoyl, 2-chlorobenzoyl, 3-chlorobenzoyl, 4-chlorobenzoyl, 3-cyanobenzoyl, 4-cyanobenzoyl, 2,3-dichlorobenzoyl, 3,4-dichlorobenzoyl, 2,5-dichlorobenzoyl, 2,4-dimethylbenzoyl, 3,5-dimethylbenzoyl, 2-fluoro-3-methylbenzoyl, 2-fluorobenzoyl, 3-fluorobenzoyl, 4-fluorobenzoyl, 2-methoxybenzoyl, 3-methoxybenzoyl, 4-methoxybenzoyl, 2-methylbenzoyl, 3-methylbenzoyl, 4-(methylthio)benzoyl, 4-nitrobenzoyl, 4-(trifluoromethoxy)benzoyl, and 3-(trifluoromethyl)benzoyl.

The term “aryloxy” as used herein, means an aryl group, as defined herein, appended to the parent molecular moiety through an oxygen atom. Representative examples of aryloxy include, but are not limited to, 2-bromophenoxy, 2-chlorophenoxy, 3-chlorophenoxy, 4-chlorophenoxy, 4-cyanophenoxy, 2,3-dichlorophenoxy, 3,4-dichlorophenoxy, 2,5-dichlorophenoxy, 2,4-dimethylphenoxy, 3,5-dimethylphenoxy, 2-fluoro-3-methylphenoxy, 2-fluorophenoxy, 3-fluorophenoxy, 4-fluorophenoxy, 2-methoxyphenoxy, 3-methoxyphenoxy, 4-methoxyphenoxy, 2-methylphenoxy, 3-methylphenoxy, 4-(methylthio)phenoxy, 3-nitrophenoxy, 4-nitrophenoxy, 4-(trifluoromethoxy)phenoxy, and 3-(trifluoromethyl)phenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-(2-bromophenoxy)ethyl, 2-(2-chlorophenoxy)ethyl, 3-chlorophenoxymethyl, 4-chlorophenoxymethyl, 4-cyanophenoxymethyl, 2,3-dichlorophenoxymethyl, 3,4-dichlorophenoxymethyl, 2,5-dichlorophenoxymethyl, 2,4-dimethylphenoxymethyl, 3,5-dimethylphenoxymethyl, 2-fluoro-3-methylphenoxymethyl, 2-fluorophenoxymethyl, 3-fluorophenoxymethyl, 4-fluorophenoxymethyl, 2-methoxyphenoxymethyl, 3-methoxyphenoxymethyl, 4-methoxyphenoxymethyl, 2-methylphenoxymethyl, 3-methylphenoxymethyl, 4-(methylthio)phenoxymethyl, 3-nitrophenoxymethyl, 4-nitrophenoxymethyl, 4-(trifluoromethoxy)phenoxymethyl, and 3-(trifluoromethyl)phenoxymethyl.

The term “arylsulfonyl” as used herein, means an aryl group, as defined herein, appended to the parent molecular moiety through a sulfonyl group, as defined herein. Representative examples of arylsulfonyl include, but are not limited to, phenylsulfonyl, naphthylsulfonyl, 2-bromophenylsulfonyl, 2-chlorophenylsulfonyl, 3-chlorophenylsulfonyl, 4-chlorophenylsulfonyl, 3-cyanophenylsulfonyl, 4-cyanophenylsulfonyl, 2,3-dichlorophenylsulfonyl, 3,4-dichlorophenylsulfonyl, 2,5-dichlorophenylsulfonyl, 2,4-dimethylphenylsulfonyl, 3,5-dimethylphenylsulfonyl, 2-fluoro-3-methylphenylsulfonyl, 2-fluorophenylsulfonyl, 3-fluorophenylsulfonyl, 4-fluorophenylsulfonyl, 2-methoxyphenylsulfonyl, 3-methoxyphenylsulfonyl, 4-methoxyphenylsulfonyl, 2-methylphenylsulfonyl, 3-methylphenylsulfonyl, 4-(methylthio)phenylsulfonyl, 4-nitrophenylsulfonyl, 4-(trifluoromethoxy)phenylsulfonyl, and 3-(trifluoromethyl)phenylsulfonyl.

The term “arylthio” as used herein, means an aryl group, as defined herein, appended to the parent molecular moiety through a sulfur atom. Representative examples of arylthio include, but are not limited to, 2-bromophenylthio, 2-chlorophenylthio, 3-chlorophenylthio, 4-chlorophenylthio, 4-cyanophenylthio, 2,3-dichlorophenylthio, 3,4-dichlorophenylthio, 2,5-dichlorophenylthio, 2,4-dimethylphenylthio, 3,5-dimethylphenylthio, 2-fluoro-3-methylphenylthio, 2-fluorophenylthio, 3-fluorophenylthio, 4-fluorophenylthio, 2-methoxyphenylthio, 3-methoxyphenylthio, 4-methoxyphenylthio, 2-methylphenylthio, 3-methylphenylthio, 4-(methylthio)phenylthio, 3-nitrophenylthio, 4-nitrophenylthio, 4-(trifluoromethoxy)phenylthio, and 3-(trifluoromethyl)phenylthio.

The term “arylthioalkyl” as used herein, means an arylthio group, as defined herein, appended to the parent molecular moiety through an alkyl group, as defined herein. Representative examples of arylthioalkyl include, but are not limited to, 2-bromophenylthiomethyl, 2-chlorophenylthiomethyl, 3-chlorophenylthiomethyl, 4-chlorophenylthiomethyl, 4-cyanophenylthiomethyl, 2,3-dichlorophenylthiomethyl, 3,4-dichlorophenylthiomethyl, 2,5-dichlorophenylthiomethyl, 2,4-dimethylphenylthiomethyl, 3,5-dimethylphenylthiomethyl, 2-fluoro-3-methylphenylthiomethyl, 2-fluorophenylthiomethyl, 3-fluorophenylthiomethyl, 4-fluorophenylthiomethyl, 2-methoxyphenylthiomethyl, 3-methoxyphenylthiomethyl, 4-methoxyphenylthiomethyl, 2-methylphenylthiomethyl, 3-methylphenylthiomethyl, 4-(methylthio)phenylthiomethyl, 3-nitrophenylthiomethyl, 4-nitrophenylthiomethyl, 4-(trifluoromethoxy)phenylthiomethyl, and 3-(trifluoromethyl)phenylthiomethyl.

The term “carbonyl” as used herein, means a —C(═O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cycloalkyl group, as defined herein, which contains 1 or 2 double bonds. The cycloalkenyl groups of the present invention may exist as a monocyclic, bicyclic, or tricyclic ring system. Monocyclic ring systems are exemplified by a cyclic hydrocarbon group containing from 3 to 8 carbon atoms containing 1 or 2 double bonds. Examples of monocyclic ring systems include cyclobutenyl, cyclopentenyl, cyclohexenyl, cycloheptenyl, and cyclooctenyl. Bicyclic fused cycloalkenyl ring systems are exemplified by a cycloalkenyl group, as defined herein, appended to the parent molscular moiety, which is fused to a phenyl group. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptene, bicyclo[2.2.1]heptene, bicyclo[2.2.2]octene, bicyclo[3.2.2]nonene, bicyclo[3.3.1 ]nonene, and bicyclo[4.2.1]nonene. Tricyclic ring systems are exemplified by a bicyclic ring system in which two nonadjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonene and tricyclo[3.3.1.1^{3,7}]decene.

The cycloalkenyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, NZ₅Z₆ and (NZ₅Z₆)alkyl.

The term “cycloalkenylalkoxy” as used herein, means a cycloalkenyl group, as defined herein, appended to the parent molecular moiety through an alkoxy group, as defined herein. Representative examples of cycloalkenylalkoxy include, but are not limited to, cyclopropenylmethoxy, cyclobutenylnethoxy, cyclopentenylmethoxy, cyclohexenylmethoxy, cycloheptenylmethoxy, and cyclooctenylmethoxy.

The term “cycloalkenylalkoxyalkyl” as used herein, means a cycloalkenylalkoxy group, as defined herein, appended to the parent molecular moiety through an alkyl group, as defined herein. Representative examples of cycloalkenylalkoxyalkyl include, but are not limited to, cyclopropenylmethoxymethyl, cyclobutenylmethoxymethyl, cyclopentenylmethoxymethyl, cyclohexenylmethoxymethyl, cycloheptenylmethoxymethyl, and cyclooctenylmethoxymethyl.

The term “cycloalkenylalkyl” as used herein, means a cycloalkenyl group, as defined herein, appended to the parent molecular moiety through an alkyl group, as defined herein. Representative examples of cycloalkenylalkyl include, but are not limited to, cyclopropenylmethyl, cyclobutenylmethyl, cyclopentenylmethyl, cyclohexenylmethyl, cycloheptenylmethyl, and cyclooctenylmethyl.

The term “cycloalkenylalkylthio” as used herein, means a cycloalkenylalkyl group, as defined herein, appended to the parent molecular moiety through a sulfur atom. Representative examples of cycloalkenylalkylthio include, but are not limited to, cyclopropenylmethylthio, cyclobutenylmethylthio, cyclopentenylmethylthio, cyclohexenylmethylthio, cycloheptenylmethylthio, and cyclooctenylmethylthio.

The term “cycloalkenylalkylthioalkyl” as used herein, means a cycloalkenylalkylthio group, as defined herein, appended to the parent molecular moiety through an alkyl group, as defined herein. Representative examples of cycloalkenylalkylthioalkyl include, but are not limited to, cyclopropenylmethylthiomethyl, cyclobutenylmethylthiomethyl, cyclopentenylmethylthiomethyl, cyclohexenylmethylthiomethyl, cycloheptenylmethylthiomethyl, and cyclooctenylmethylthiomethyl.

The term “cycloalkenyloxy” as used herein, means a cycloalkenyl group, as defined herein, appended to the parent molecular moiety through an oxygen atom. Representative examples of cycloalkenyloxy include, but are not limited to, cyclopropenybxy, cyclobutenyloxy, cyclopentenyloxy, cyclohexenyloxy, cycloheptenyloxy, and cyclooctenyloxy.

The term “cycloalkenyloxyalkyl” as used herein, means a cycloalkenyloxy group, as defined herein, appended to the parent molecular moiety through an alkyl group, as defined herein. Representative examples of cycloalkenyloxyalkyl include, but are not limited to, cyclopropenyloxymethyl, cyclobutenyloxymethyl, cyclopentenyloxymethyl, cyclohexenyloxymethyl, cycloheptenyloxymethyl, and cyclooctenyloxymethyl.

The term “cycloalkenylthio” as used herein, means a cycloalkenyl group, as defined herein, appended to the parent molecular moiety through a sulfur atom. Representative examples of cycloalkenylthio include, but are not limited to, cyclopropenylthio, cyclobutenylthio, cyclopentenylthio, cyclohexenylthio, cycloheptenylthio, and cyclooctenylthio.

The term “cycloalkenylthioalkyl” as used herein, means a cycloalkenylthio group, as defined herein, appended to the parent molecular moiety through an alkyl group, as defined herein. Representative examples of cycloalkenylthioalkyl include, but are not limited to, cyclopropenylthiomethyl, cyclobutenylthiomethyl, cyclopentenylthiomethyl, cyclohexenylthiomethyl, cycloheptenylthiomethyl, and cyclooctenylthiomethyl.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic fused ring systems are exemplified by a cycloalkyl group appended to the parent molecular moiety, which is fused to an additional cycloalkyl group, as defined herein, a phenyl group, a heteroaryl, as defined herein, or a heterocycle as defined herein. Tricyclic fused ring systems are exemplified by a cycloalkyl bicyclic fused ring system fused to an additional cycloalkyl group, as defined herein, a phenyl group, a heteroaryl, as defined herein, or a heterocycle as defined herein. The additional fused cycloalkyl group may be substituted but may not be fused to another ring. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, NZ₅Z₆ and (NZ₅Z₆)alkyl.

The term “cycloalkylalkoxy” as used herein, means a cycloalkyl group, as defined herein, appended to the parent molecular moiety through an alkoxy group, as defined herein. Representative examples of cycloalkylalkoxy include, but are not limited to, cyclopropylmethoxy, cyclobutylmethoxy, cyclopentylmethoxy, cyclohexylmethoxy, 2-cyclohexylethoxy, cycloheptylmethoxy, and cyclooctylmethoxy.

The term “cycloalkylalkoxyalkyl” as used herein, means a cycloalkylalkoxy group, as defined herein appended to the parent molecular moiety through an alkyl group, as defined herein. Representative examples of cycloalkoxyalkyl include, but are not limited to, cyclopropylmethoxymethyl, cyclobutylmethoxymethyl, cyclopentylmethoxymethyl, cyclohexylmethoxymethyl, (2-cyclohexylethoxy)methyl, cycloheptylmethoxymethyl, and cyclooctylmethoxymethyl.

The term “cycloalkylalkyl” as used herein, means a cycloalkyl group, as defined herein, appended to the parent molecular moiety through an alkyl group as defined herein. Representative examples of cycloalkylalkyl include, but are not limited to, cyclopropylmethyl, cyclobutylmethyl, cyclopentylmethyl, cyclohexylmethyl, 2-cyclohexylethyl, cycloheptylmethyl, and cyclooctylmethyl.

The term “cycloalkylalkylthio” as used herein, means a cycloalkylalkyl group, as defined herein, appended to the parent molecular moiety through a sulfur atom. Representative examples of cycloalkylalkylthio include, but are not limited to, cyclopropylmethylthio, cyclobutylmethylthio, cyclopentylmethylthio, cyclohexylmethylthio, 2-cyclohexylethylthio, cycloheptylmethylthio, and cyclooctylmethylthio.

The term “cycloalkylalkylthioalkyl” as used herein, means a cycloalkylalkylthio group, as defined herein, appended to the parent molecular moiety through an alkyl group, as defined herein. Representative examples of cycloalkylalkylthioalkyl include, but are not limited to, cyclopropylmethylthiomethyl, cyclobutylmethylthiomethyl, cyclopentylmethylthiomethyl, cyclohexylmethylthiomethyl, 2-cyclohexylethylthiomethyl, cycloheptylmethylthiomethyl, and cyclooctylmethylthiomethyl.

The term “cycloalkylcarbonyl” as used herein, means a cycloalkyl group, as defined herein, appended to the parent molecular moiety through a carbonyl group as defined herein. Representative examples of cycloalkylcarbonyl include, but are not limited to, cyclopropylcarbonyl, cyclobutylcarbonyl, cyclopentylcarbonyl, cyclohexylcarbonyl, cycloheptylcarbonyl, and cyclooctylcarbonyl.

The term “cycloalkyloxy” as used herein, means a cycloalkyl group, as defined herein, appended to the parent molecular moiety through an oxygen atom, examples of cycloalkyloxy include cyclopropyloxy, cyclobutyloxy, cyclopentyloxy, cyclohexyloxy, cycloheptyloxy, and cyclooctyloxy.

The term “cycloalkyloxyalkyl” as used herein, means a cycloalkyloxy group, as defined herein, appended to the parent molecular moiety through an alkyl group, as defined herein. Representative examples of cycloalkyloxyalkyl include, but are not limited to, cyclopropyloxymethyl, cyclobutyloxymethyl, cyclopentyloxymethyl, cyclohexyloxymethyl, cycloheptyloxymethyl, and cyclooctyloxymethyl.

The term “cycloalkylthio” as used herein, means a cycloalkyl group, as defined herein, appended to the parent molecular moiety through a sulfur atom, examples of cycloalkylthio include cyclopropylthio, cyclobutylthio, cyclopentylthio, cyclohexylthio, cycloheptylthio, and cyclooctylthio.

The term “cycloalkylthioalkyl” as used herein, means a cycloalkylthio group, as defined herein, appended to the parent molecular moiety through an alkyl group, as defined herein. Representative examples of cycloalkylthioalkyl include cyclopropylthiomethyl, cyclobutylthiomethyl, cyclopentylthiomethyl, cyclohexylthiomethyl, cycloheptylthiomethyl, and cyclooctylthiomethyl.

The term “formyl,” as used herein, means a —C(O)H group.

The term “formylalkyl” as used herein, means a fornyl group, as defined herein, appended to the parent molecular moiety through an alkyl group as defined herein. Representative examples of formylalkyl include, but are not limited to, cyclopropylmethyl, cyclobutylmethyl, cyclopentylmethyl, cyclohexylmethyl, 2-oxoethyl, 3-oxopropyl, and 4-oxobutyl.

The term “halo” or “halogen,” as used herein, means —Cl, —Br, —I or —F.

The term “haloalkoxy,” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, pentafluoroethoxy, and 2-chloro-3-fluoropentoxy.

The term “haloalkyl,” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylcarbonyl,” as used herein, means a haloalkyl group, as defined herein, appended to the parent molecular moiety through a carbonyl group, as defined herein. Representative examples of haloalkylcarbonyl include, but are not limited to, chloromethylcarbonyl, 2-fluoroethylcarbonyl, trifluoromethylcarbonyl, pentafluoroethylcarbonyl, and 2-chloro-3-fluoropentylcarbonyl.

The term “haloalkylsulfonyl,” as used herein, means a haloalkyl group, as defined herein, appended to the parent molecular moiety through a sulfonyl group, as defined herein. Representative examples of haloalkylsulfonyl include, but are not limited to, chloromethylsulfonyl, 2-fluoroethylsulfonyl, trifluoromethylsulfonyl, pentafluoroethylsulfonyl, and 2-chloro-3-fluoropentylsulfonyl.

The term “heteroaryl,” as used herein, means an aromatic monocyclic ring or an aromatic bicyclic ring. The aromatic monocyclic rings are five or six membered rings wherein 1, 2, 3, or 4 atoms are independently a member selected from the group consisting of N, O, and S. The five membered aromatic monocyclic rings have two double bonds and the six membered aromatic monocyclic rings have three double bonds. The heteroaryl bicyclic rings are exemplified by a heteroaryl monocyclic ring appended to the parent molecular moiety, which is fused to a phenyl group, or another heteroaryl group as herein defined. The heteroaryl monocyclic rings and the heteroaryl bicyclic rings are connected to the parent molecular moiety through a carbon or nitrogen atom. The aromatic monocyclic rings and the aromatic bicyclic rings are connected to the parent molecular moiety through a carbon or nitrogen atom. Representative examples of heteroaryl include, but are not limited to, benzimidazole, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl, and triazinyl.

The heteroaryl groups of the present invention are substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl, hydroxyhaloalkyl, mercapto, nitro, —NZ₅Z₆ and (NZ₅Z₆)alkyl.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylnethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-yhnethoxy.

The term “heteroarylalkoxyalkyl” as used herein, means a heteroarylalkoxy group, as defined herein, appended to the parent molecular moiety through an alkyl group, as defined herein. Representative examples of heteroarylalkoxyalkyl include, but are not limited to, fur-3-ylmethoxymethyl, 1H-imidazol-2-ylmethoxymethyl, 1H-imidazol-4-ylmethoxymethyl, pyridin-3-ylmethoxymethyl, 6-chloropyridin-3-ylmethoxymethyl, pyridin-4-ylmethoxymethyl, (6-(trifluoromethyl)pyridin-3-yl)methoxymethyl, (6-(cyano)pyridin-3-yl)methoxymethyl, (2-(cyano)pyridin-4-yl)methoxymethyl, (5-(cyano)pyridin-2-yl)methoxymethyl, (2-(chloro)pyridin-4-yl)methoxymethyl, pyrimidin-5-ylmethoxymethyl, 2-(pyrimidin-2-yl)propoxymethyl, thien-2-ylmethoxymethyl, and thien-3-ylmethoxymethyl.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-yl)methylthio, pyrimidin-5-ylmethylthio, 2-(pyrimidin-2-yl)propylthio, thien-2-ylmethylthio, and thien-3-ylmethylthio.

The term “heteroarylalkylthioalkyl” as used herein, means a heteroarylalkylthio group, as defined herein, appended to the parent molecular moiety through an alkyl group, as defined herein. Representative examples of heteroarylalkylthioalkyl include, but are not limited to, fur-3-ylmethylthiomethyl, 1H-imidazol-2-ylmethylthiomethyl, 1H-imidazol-4-ylmethylthiomethyl, pyridin-3-ylmethylthiomethyl, 6-chloropyridin-3-ylmethylthiomethyl, pyridin-4-ylmethylthiomethyl, (6-(trifluoromethyl)pyridin-3-yl)methylthiomethyl, (6-(cyano)pyridin-3-yl)methylthiomethyl, (2-(cyano)pyridin-4-yl)methylthiomethyl, (5-(cyano)pyridin-2-yl)methylthiomethyl, (2-(chloro)pyridin-4-yl)methylthiomethyl, pyrimidin-5-ylmethylthiomethyl, 2-(pyrimidin-2-yl)propylthiomethyl, thien-2-ylmethylthiomethyl, and thien-3-ylmethylthiomethyl.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, fur-3-yloxymethyl, 1H-imidazol-2-yloxymethyl, 1H-imidazol-4-yloxymethyl, pyridin-3-yloxymethyl, 6-chloropyridin-3-yloxymethyl, pyridin-4-yloxymethyl, (6-(trifluoromethyl)pyridin-3-yl)oxymethyl, (6-(cyano)pyridin-3-yl)oxymethyl, (2-(cyano)pyridin-4-yl)oxymethyl, (5-(cyano)pyridin-2-yl)oxymethyl, (2-(chloro)pyridin-4-yl)oxymethyl, pyrimidin-5-yloxymethyl, pyrimidin-2-yloxymethyl, thien-2-yloxymethyl, and thien-3-yloxymethyl.

The term “heteroarylthio” as used herein, means a heteroaryl group, as defined herein, appended to the parent molecular moiety through a sulfur atom. Representative examples of heteroarylthio include, but are not limited to, fur-3ylthio, 1H-imidazol-2-ylthio, 1H-imidazol-4-ylthio, pyridin-3-ylthio, 6-chloropyridin-3-ylthio, pyridin-4-ylthio, (6-(trifluoromethyl)pyridin-3-yl)thio, (6-(cyano)pyridin-3-yl)thio, (2-(cyano)pyridin-4-yl)thio, (5-(cyano)pyridin-2-yl)thio, (2-(chloro)pyridin-4-yl)thio, pyrimidin-5-ylthio, pyrimidin-2-ylthio, thien-2-ylthio, and thien-3-ylthio.

The term “heteroarylthioalkyl” as used herein, means a heteroarylthio group, as defined herein, appended to the parent molecular moiety through an alkyl group, as defined herein. Representative examples of heteroarylthioalkyl include, but are not limited to, fur-3-ylthiomethyl, 1H-imidazol-2-ylthiomethyl, 1H-imidazol-4-ylthiomethyl, pyridin-3-ylthiomethyl, 6-chloropyridin-3-ylthiomethyl, pyridin-4-ylthiomethyl, (6-(trifluoromethyl)pyridin-3-yl)thiomethyl, (6-(cyano)pyridin-3-yl)thiomethyl, (2-(cyano)pyridin-4-yl)thiomethyl, (5-(cyano)pyridin-2-yl)thiomethyl, (2-(chloro)pyridin-4-yl)thiomethyl, pyrimidin-5-ylthiomethyl, pyrimidin-2-ylthiomethyl, thien-2-ylthiomethyl, and thien-3-ylthiomethyl.

The term “heterocycle,” as used herein, means a non-aromatic monocyclic ring or a nom aromatic bicyclic ring. The non-aromatic monocyclic ring is a three, four, five, six, seven, or eight membered ring containing 1 or 2 heteroatoms independently a member selected from the group consisting of N, O, and S. The three membered rings have zero double bonds. The four and five membered rings have zero or one double bond. The six membered rings have zero, one, or two double bonds. The seven and eight membered rings have zero, one, two, or three double bonds. The bicyclic heterocycle rings are composed of a nonaromatic heterocyclic monocyclic ring appended to the parent molecular moiety, which is fused to a cycloalkyl group, as defined herein, or a phenyl group. Alternatively, bicyclic heterocyclic rings are composed of a non-aromatic heterocyclic monocyclic ring fused to another non-aromatic heterocyclic monocyclic ring. The heterocycle groups of the present invention can be attached to the parent molecular moiety through a carbon atom or a nitrogen atom. Representative examples of heterocycle include, but are not limited to, azetidinyl, 1,3-benzodioxolyl, 1,3-benzodioxol-4-yl, hexahydro-1H-azepinyl, hexahydroazocin-(2H)-yl, morpholinyl, piperazinyl, piperidinyl, pyrrolidinyl, tetrahydrofuranyl, tetrahydrofuran-2-yl, tetrahydrofuran-3-yl, tetrahydro-2H-pyranyl, tetrahydro-2H-pyran-2-yl, tetrahydro-2H-pyran-4-yl, tetrahydrothienyl, tetrahydrothien-2-yl, and tetrahydrothien-3-yl, and thiomorpholinyl.

The heterocycles of the present invention are substituted with 0, 1, 2, 3, or 4 substituents independently a member selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, halogen, hydroxy, hydroxyalkyl, mercapto, nitro, oxo, Z₅Z₆N— and (Z₅Z₆N)alkyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 1,3-benzodioxol-4-ylmethoxy, pyridin-3-ylmethoxy, 2-pyrimidin-2-ylpropoxy, tetrahydrofuran-2-ylmethoxy, tetrahydrofuran-3-ylmethoxy, tetrahydro-2H-pyran-2-ylmethoxy, tetrahydro-2H-pyran-4-ylmethoxy, tetrahydrothien-2-ylmethoxy, and tetrahydrothien-3-ylmethoxy.

The term “heterocyclealkoxyalkyl” as used herein, means a heterocyclealkoxy group, as defined herein, appended to the parent molecular moiety through an alkyl group, as defined herein. Represent-ative examples of heterocyclealkoxyalkyl-include, but are not limited to, 1,3-benzodioxol-4-ylmethoxymethyl, pyridin-3-ylmethoxymethyl, 2-pyrimidn-2-ylpropoxymethyl, tetrahydrofuran-2—,ylmethoxymethyl, tetrahydrofuran-3-ylmethoxymethyl, tetrahydro-2H-pyran-2-ylmethoxymethyl, tetrahydro-2H-pyran-4ylmethoxymethyl, tetrahydrothien-2-ylmethoxymethyl, and tetrahydrothien-3-ylmethoxymethyl.

The term “heterocyclealkyl” as used herein, means a heterocycle group, as defined herein, appended to the parent molecular moiety through an alkyl group, as defined herein. Representative examples of heterocyclealkyl include, but are not limited to, 1,3-benzodioxol-4-ylmethyl, pyridin-3-ylmethyl, 2-pyrimidin-2-ylpropyl, tetrahydrofuran-2-ylmethyl, tetrahydrofuran-3-ylmethyl, tetrahydro-2H-pyran-2-ylmethyl, tetrahydro-2H-pyran-4-ylmethyl, tetrahydrothien-2-ylmethyl, and tetrahydrothien-3-ylmethyl.

The term “heterocyclealkylthio” as used herein, means a heterocyclealkyl group, as defined herein, appended to the parent molecular moiety through a sulfur atom. Representative examples of heterocyclealkylthio include, but are not limited to, 1,3-benzodioxol-4-ylmethylthio, pyridin-3-ylmethylthio, 2-pyrimidin-2-ylpropylthio, tetrahydrofuran-2-ylmethylthio, tetrahydroftiran-3-ylmethylthio, tetrahydro-2H-pyran-2-ylmethylthio, tetrahydro-2H-pyran-4-ylmethylthio, tetrahydrothien-2-ylmethylthio, and tetrahydrothien-3-ylmethylthio.

The term “heterocyclealkylthioalkyl” as used herein, means a heterocyclealkylthio group, as defined herein, appended to the parent molecular moiety through an alkyl group, as defined herein. Representative examples of heterocyclealkylthioalkyl include, but are not limited to, 1,3-benzodioxol-4-ylmethylthiomethyl, pyridin-3-ylmethylthiomethyl, 2-pyrimidin-2-ylpropylthiomethyl, tetrahydrofuran-2-ylmethylthiomethyl, tetrahydrofuran-3-ylmethylthiomethyl, tetrahydro-2H-pyran-2-ylmethylthiomethyl, tetrahydro-2H-pyran-4-ylmethylthiomethyl, tetrahydrothien-2-ylmethylthiomethyl, and tetrahydrothien-3-ylmethylthiomethyl.

The term “heterocyclecarbonyl” as used herein, means a heterocycle group, as defined herein, appended to the parent molecular moiety through a carbonyl group, as defined herein. Representative examples of heterocyclecarbonyl include, but are not limited to, 1,3-benzodioxol-4-ylcarbonyl, pyridin-3-ylcarbonyl, pyrimidin-2-ylcarbonyl, tetrahydrofuran-2-ylcarbonyl, tetrahydroftiran-3-ylcarbonyl, tetrahydro-2H-pyran-2-ylcarbonyl, tetrahydro-2H-pyran-4-ylcarbonyl, tetrahydrothien-2-ylcarbonyl, and tetrahydrothien-3-ylcarbonyl.

The term “heterocycleoxy” as used herein, means a heterocycle group, as defined herein, appended to the parent molecular moiety through an oxygen atom. Representative examples of heterocycleoxy include, but are not limited to, 1,3-benzodioxol-4-yloxy, pyridin-3-yloxy, 2-pyrimidin-2-yloxy, tetrahydroftiran-2-yloxy, tetrahydrofuran-3-yloxy, tetrahydro-2H-pyran-2-yloxy, tetrahydro-2H-pyran-4-yloxy, tetrahydrothien-2-yloxy, and tetrahydrothien-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, 1,3-benzodioxol-4-yloxymethyl, pyridin-3-yloxymethyl, 2-pyrimidin-2-yloxymethyl, tetrahydrofuran-2-yloxymethyl, tetrahydrofuran-3-yloxymethyl, tetrahydro-2H-pyran-2-yloxymethyl, tetrahydro-2H-pyran-4-yloxymethyl, tetrahydrothien-2-yloxymethyl, and tetrahydrothien-3-yloxymethyl.

The term “heterocyclethio” as used herein, means a heterocycle group, as defined herein, appended to the parent molecular moiety through a sulfur atom. Representative examples of heterocyclethio include, but are not limited to, 1,3-benzodioxol-4-ylthio, pyridin-3-ylthio, 2-pyrimidin-2-ylthio, tetrahydrofuran-2-ylthio, tetrahydrofuran-3-ylthio, tetrahydro-2H-pyran-2-ylthio, tetrahydro-2H-pyran-4-ylthio, tetrahydrothien-2-ylthio, and tetrahydrothien-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, 1,3-benzodioxol-4-ylthiomethyl, pyridin-3-ylthiomethyl, 2-pyrimidin-2-ylthiomethyl, tetrahydrofuran-2-ylthiomethyl, tetrahydrofuran-3-ylthiomethyl, tetrahydro-2H-pyran-2-ylthiomethyl, tetrahydro-2H-pyran-4-ylthiomethyl, tetrahydrothien-2-ylthiomethyl, and tetrahydrothien-3-ylthiomethyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, 2-hydroxyethyl, 2-hydroxypropyl, 1,2-dihydroxypropyl, 3-hydroxybutyl and the like.

The term “hydroxyhaloalkyl” as used herein, means at least one hydroxy group, as defined herein, appended to the parent molecular moiety through a haloalkyl group, as defined herein.

The term “R_{A}R_{B}N” as used herein, means two groups, R_{A} and R_{B}, which are appended to the parent molecular moiety through a nitrogen atom. R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl. Representative examples of R_{A}R_{B}N include, but are not limited to, amino, methylamino, acetylamino, and acetylmethylamino.

The term “R_{C}R_{D}N—” as used herein, means two groups, R_{C} and RD, which are appended to the parent molecular moiety through a nitrogen atom. R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl. Representative examples of R_{C}R_{D}N— include, but are not limited to, amino, methylamino, acetylamino, acetylmethylamino, benzylamino, benzyl(methyl)amino, dimethylamino, methylamino, ethylamino, diethylamino, cyclohexylamino, cyclohexylmethylamino, and phenylamino.

The term “(R_{C}R_{D}N)alkyl” as used herein, means a R_{C}R_{D}N— group, as defined herein, appended to the parent molecular moiety through an alkyl group, as defined herein. Representative examples of (R_{C}R_{D}N)alkyl include, but are not limited to, aminomethyl, methylaminomethyl, acetylaminomethyl, acetylmethylaminomethyl, benzylaminomethyl, benzyl(methyl)amino, dimethylaminomethyl, ethylaminomethyl, diethylaminomethyl, cyclohexylaminomethyl, cyclohexylmethylaminomethyl, butylaminomethyl, 3-methylphenylaminomethyl, and phenylaminomethyl.

The term “R_{E}R_{F}N—” as used herein, means two groups, R_{E} and R_{F}, which are appended to the parent molecular moiety through a nitrogen atom. R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (Z₁Z₂N)alkyl, and (Z₁Z₂N)carbonyl. Representative examples of R_{E}R_{F}N— include, but are not limited to, amino, methylamino, acetylamino, acetylmethylamino, benzylamino, butylamino, 3-methylphenylamino, and phenylamino.

The term “(R_{E}R_{F}N)alkyl” as used herein, means a R_{E}R_{F}N— group, as defined herein, appended to the parent molecular moiety through an alkyl group, as defined herein. Representative examples of (R_{E}R_{F}N)alkyl include, but are not limited to, aminomethyl, methylaminomethyl, acetylaminomethyl, acetylmethylaminomethyl, benzylaminomethyl, butylaminomethyl, 3-methylphenylaminomethyl, and phenylaminomethyl.

The term “(R_{E}R_{F}N)carbonyl” as used herein, means a R_{E}R_{F}N— group, as defined herein, appended to the parent molecular moiety through a carbonyl group, as defined herein. Representative examples of (R_{E}R_{F}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, and phenylaminocarbonyl.

The term “(R_{E}R_{F}N)carbonylalkenyl” as used herein, means a (R_{E}R_{F}N)carbonyl group, as defined herein, appended to the parent molecular moiety through an alkenyl group, as defined herein.

The term “(R_{E}R_{F}N)carbonylalkyl” as used herein, means a (R_{E}R_{F}N)carbonyl group, as defined herein, appended to the parent molecular moiety through an alkyl group, as defined herein. Representative examples of (R_{E}R_{F}N)carbonylalkyl include, but are not limited to, aminocarbonylmethyl, methylaminocarbonylmethyl, acetylaminocarbonylmethyl, acetylmethylaminocarbonylmethyl, 2-(benzylaminocarbonyl)ethyl, 2-(butylaminocarbonyl)ethyl, 2-(3-methylphenylaminocarbonyl)ethyl, and 2-(phenylaminocarbonyl)ethyl.

The term “(R_{E}R_{F}N)sulfonyl” as used herein, means a R_{E}R_{F}N— group, as defined herein, appended to the parent molecular moiety through a sulfonyl group, as defined herein. Representative examples of (R_{E}R_{F}N)sulfonyl include, but are not limited to, aminosulfonyl and dimethylaaminosulfonyl.

The term “(R_{E}R_{F}N)sulfonylalkyl” as used herein, means a (R_{E}R_{F}N)sulfonyl group, as defined herein, appended to the parent molecular moiety through an alkyl group, as defined herein. Representative examples of (R_{E}R_{F}N)sulfonylalkyl include, but are not limited to, aminosulfonylmethyl and dimethylaminosulfonylmethyl.

The term “R_{G}R_{H}N—” as used herein, means two groups, R_{G} and R_{H}, which are appended to the parent molecular moiety through a nitrogen atom. R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (Z₃Z₄N)alkyl, and (Z₃Z₄N)carbonyl. Representative examples of R_{G}R_{H}N— include, but are not limited to, amino, methylamino, acetylamino, acetylmethylamino, benzylamino, (2-(benzyloxy)ethyl)amino, butylamino, cyclohexylmethylamino, cycloheptylamino, dimethylamino, ethylamino, (1-ethylpropyl)amino, isobutylamino, 3-methylphenylamino, neopentylamino, 4-nitrobenzylamino, 4-nitrophenylamino, (2-(4-nitrophenyl)ethyl)amino, phenylamino, propylamino, propylaminocarbonylamino, propionylamino, (1,3-benzodioxol-4-ylmethyl)amino, (butoxyacetyl)amino, 4-chlorobenzylamino, (4-chlorobenzyl)acetylamino, (4-chlorobenzyl)formylamino, (4-chlorobenzyl)methylamino, (1-(4-chlorophenyl)ethyl)amino, (2-(4-chlorophenyl)ethyl)amino, 2-chloropyridin-4-ylmethylamino, 6-chloropyridin-3-ylmethylamino, cyclopropylmethylamino, 3,4-dichlorobenzylamino, 4-cyanobenzylamino, (4-cyanobenzyl)methylamino, 4-cyanophenylamino, (1-(4-cyanophenyl)ethyl)amino, 2-(cyano)pyridin-4-ylmethylamino, 5-(cyano)pyridin-2-ylmethylamino, 6-(cyano)pyridin-3-ylmethylamino, (2-(tert-butoxycarbonylamino)ethyl)amino, fur-3-ylmethylamino, 4-methoxybenzylamino, tetrahydrofuran-3-ylmethylamino, tetrahydro-2H-pyran-4-ylmethylamino, (4-chlorophenylcarbonyl)amino, pyridin-2-ylmethylamino, pyridin-3-ylmethylamino, pyridin-4-ylmethylamino, (1-(pyridin-4-yl)ethyl)amino, pyrimidin-5-ylmethylamino, 1H-imidazol-4-ylmethylamino, 1H-imidazol-2-ylmethylamino, thien-2-ylmethylamino, thien-3-ylmethylamino, 4-(trifluoromethoxy)benzylamino, and 6-(trifluoromethyl)pyridin-3-ylmethylamino.

The term “(R_{G}R_{H}N)alkyl” as used herein, means a R_{G}R_{H}N group, as defined herein, appended to the parent molecular moiety through an alkyl group, as defined herein. Representative examples of (R_{G}R_{H}N)alkyl include, but are not limited to, aminomethyl, methylaminomethyl, acetylaminomethyl, acetylmethylaminomethyl, benzylaminomethyl, (2-(benzyloxy)ethyl)aminomethyl, butylaminomethyl, cyclohexylmethylaminomethyl, cycloheptylaminomethyl, dimethylaminomethyl, ethylaminomethyl, (1-ethylpropyl)aminomethyl, isobutylaminomethyl, 3-methylphenylaminomethyl, neopentylaminomethyl, 4-nitrobenzylaminomethyl, 4-nitrophenylaminomethyl, (2-(4-nitrophenyl)ethyl)aminomethyl, phenylaminomethyl, propylaminomethyl, propylaminocarbonylaminomethyl, propionylaminomethyl, (1,3-benzodioxol-4-ylmethyl)aminomethyl, (butoxyacetyl)aminomethyl, 4-chlorobenzylaminomethyl, (4-chlorobenzyl)acetylaminomethyl, (4-chlorobenzyl)formylaminomethyl, (4-chlorobenzyl)methylaminomethyl, (1-(4-chlorophenyl)ethyl)aminomethyl, (2-(4-chlorophenyl)ethyl)aminomethyl, 2-chloropyridin-4-ylmethylaminomethyl, 6-chloropyridin-3-ylmethylaminomethyl, cyclopropylmethylaminomethyl, 3,4-dichlorobenzylaminomethyl, 4-cyanobenzylaminomethyl, (4-cyanobenzyl)methylaminomethyl, 4-cyanophenylaminomethyl, (1-(4-cyanophenyl)ethyl)aminomethyl, 2-(cyano)pyridin-4-ylmethylaminomethyl, 5-(cyano)pyridin-2-ylmethylaminomethyl, 6-(cyano)pyridin-3-ylmethylaminomethyl, (2-(tert-butoxycarbonylamino)ethyl)aaminomethyl, fur-3-ylmethylaminomethyl, 4-methoxybenzylaminomethyl, tetrahydrofuran-3-ylmethylaminomethyl, tetrahydro-2H-pyran-4-ylmethylaminomethyl, (4-chlorophenylcarbonyl)aminomethyl, pyridin-2-ylmethylaminomethyl, pyridin-3-ylmethylaminomethyl, pyridin-4-ylmethylaminomethyl, (1-(pyridin-4-yl)ethyl)aminomethyl, pyrimidin-5-ylmethylaminomethyl, 1H-imidazol-4-ylmethylaminomethyl, 1H-imidazol-2-ylmethylaminomethyl, thien-2-ylmethylaminomethyl, thien-3-ylmethylaminomethyl, 4-(trifluoromethoxy)benzylaminomethyl, and 6-(trifluoromethyl)pyridin-3-ylmethylaminomethyl.

The term “(R_{G}R_{H}N)carbonyl” as used herein, means a R_{G}R_{H}N— group, as defined herein, appended to the parent molecular moiety through a carbonyl group, as defined herein. Representative examples of (R_{G}R_{H}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, and phenylaminocarbonyl.

The term “(R_{G}R_{H}N)sulfonyl” as used herein, means a RCR_{H}N— group, as defined herein, appended to the parent molecular moiety through a sulfonyl group, as defined herein. Representative examples of (R_{G}R_{H}N)sulfonyl include, but are not limited to, aminosulfonyl and dimethylaminosulfonyl.

The term “R_{J}R_{K}N—” as used herein, means two groups, R_{J} and RK, which are appended to the parent molecular moiety through a nitrogen atom. R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl. Representative examples of R_{J}R_{K}N— include, but are not limited to, amino, ethylamino, benzylamino, dimethylamino, methylamino, tert-butoxycarbonylamino, and propylamino.

The term “(R_{J}R_{K}N)alkyl” as used herein, means a R_{J}R_{K}N— group, as defined herein, appended to the parent molecular moiety through an alkyl group, as defined herein. Representative examples of (R_{J}R_{K}N)alkyl include, but are not limited to, 2-aminoethyl, 2-(dimethylamino)ethyl, 2-ethylaminoethyl, and 2-(tert-butoxycarbonylamino)ethyl.

The term “(R_{J}R_{K}N)carbonyl” as used herein, means a R_{J}R_{K}N— group, as defined herein, appended to the parent molecular moiety through a carbonyl group, as defined herein. Representative examples of (R_{J}R_{K}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “(R_{J}R_{K}N)sulfonyl” as used herein, means a R_{J}R_{K}N— group, as defined herein, appended to the parent molecular moiety through a sulfonyl group, as defined herein. Representative examples of (R_{J}R_{K}N)sulfonyl include, but are not limited to, aminosulfonyl, methylaminosulfonyl, acetylaminosulfonyl, acetylmethylaminosulfonyl, benzylaminosulfonyl, butylaminosulfonyl, 3-methylphenylaminosulfonyl, propylaminosulfonyl, and phenylaminosulfonyl.

The term ” Z₁Z₂N—” as used herein, means two groups, Z₁ and Z₂, which are appended to the parent molecular moiety through a nitrogen atom. Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl. Representative examples of Z₁Z₂N— include, but are not limited to, amino, ethylamino, benzylamino, dimethylamino, methylamino, tert-butoxycarbonylamino, and propylamino.

The term “(Z₁Z₂N)alkyl” as used herein, means a Z₁Z₂N— group, as defined herein, appended to the parent molecular moiety through an alkyl group, as defined herein. Representative examples of Z₁Z₂N— include, but are not limited to, 2-aminoethyl, 2-(dimethylamino)ethyl, 2-ethylaminoethyl, and 2-(tert-butoxycarbonylamino)ethyl.

The term “(Z₁Z₂N)carbonyl” as used herein, means a Z₁Z₂N— group, as defined herein, appended to the parent molecular moiety through a carbonyl group, as defined herein. Representative examples of (Z₁Z₂N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “mercapto” as used herein, means a —SH group.

The term “nitro” as used herein, means a —NO₂ group.

The term “sulfinyl” as used herein, means a —SO— group.

The term “sulfonyl” as used herein, means a —SO₂— group.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quatemized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to the parent compounds of the present invention for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycle group. Substituents around a carbon-carbon double bond are designated as being of Z or E configuration and substituents around a cycloalkyl or heterocycle are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents. Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents which dissolve or disperse in the injectable media.

Regulation of the effects of ghrelin by the compounds of the present invention can be delayed by using a liquid suspension of crystalline or amorphous material with poor water solubility. The rate of absorption of the compounds depends upon their rate of dissolution which, in turn, depends on their crystalline form. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions which are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable nonirritating excipient which is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents which delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders that may be regulated by ghrelin are treated or prevented in a patient by administering to the patient, a therapeutically effective amount of a compound of the present invention in such an amount and for such time as is necessary to achieve the desired result. The term “therapeutically effective amount,” refers to a sufficient amount of a compound to effectively emeliorate disorders reglulated by ghrelin at a reasonable benefit/risk ratio applicable to any medical treatment. The specific therapeutically effective dose level for anyparticular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the present compounds in single or divided doses can be in amounts, for example, from 0.01 to 50 mg/kg body weight or more usually from 0.1 to 25 mg/kg body weight. In general, treatment regimens comprise administration to a patient in need of such treatment from about 10 mg to about 1000 mg of the compounds per day in single or multiple doses.

### Determination of Biological Activity

Primary Radiolabeled Ligand Competition Binding Assay

Ghrelin binding assays were performed with membrane preparations. CHO—K cells expressing human ghrelin receptor (Euroscreen) were suspended in sucrose buffer (0.25 M sucrose, 10 mM hepes pH 7.4, 1 mM PMSF, 5 kg/mL pepstain-A, 3 mM EDTA and 0.025% bacitracin) and disrupted by sonication using a vibra cell (Sonics and Materials Inc.) on 70% duty cycle in 15-second pulses on ice for 2.5 min. The homogenate was centrifuged at 60,000×g for 60 minutes and pellets were suspended in tris buffer (20 mM tris pH 7.4, 5 μg/mL pepstatin-A, 0.1 mM PMSF and 3 mM EDTA ). Binding reactions contained 1 μg membrane as determined by BCA protein assay (Pierce), 0.1 nM [¹²⁵I]-ghrelin (PerkinElmer) with or without compound addition in 100 μL of binding buffer (25 mM Hepes pH 7.4, 1 mM CaCl₂, 5 mM MgSO₄ and 0.5% protease free BSA). Incubations were carried out at room temperature for 2 hr and were terminated by filtration using Filtermate Harvester (PerkinElmer) onto GF/C filter plates (Millipore) previously soaked in 0.5% polyethylenimine for 2 hours. Bound [¹²⁵I]-ghrelin was determined by scintillation counting using Top Count NXT (PerkinElmer). The effects of compound were expressed as %inhibiton of [¹²⁵I]-ghrelin binding. Sigmoidal curves were fitted by Assay Explorer (MDL) software and IC₅₀ values were determined. GHS-R antagonist [D-Lys3]-GHRP-6 (H-His-D-Trp-D-Lys-Trp-D-Phe-Lys) was purchased from Bachem and used as a positive control.

The compounds of the present invention were found to inhibit the binding of ¹²⁵I-labeled ghrelin to ghrelin receptor with IC₅₀ in a range of about 0.0001 μM to about 10 μM in the binding assay. In a preferred range, the compounds inhibit the binding of ¹²⁵I-labeled ghrelin to ghrelin receptor with IC₅₀ in a range of about 0.0001 μM to about 1.0 μM; In a more preferred range, the compounds inhibit the binding of ¹²⁵I-labeled ghrelin to ghrelin receptor with IC₅₀ in a range of about 0.0001 μM to about 0.1 μM.

Secondary Fluorescent Calcium Indicator Assay (FLIPR)

CHO—K cells expressing human GHS receptor (Euroscreen) were plated in black 96-well plates with clear bottom (Costar) and cultured to confluency overnight in growth media (Ultra-CHO from BioWhittaker supplemented with 1% dialyzed FCS, 1% penicillin /streptomycin/fungizone, and 400 μg/mL G418 all from Life Technologies) at 37° C. in a humidified cell incubator containing 5% CO₂. Growth media was aspirated and replaced with 100 μL of Dulbecco's phosphate-buffered saline (DPBS) containing 1,000 mg/L D-glucose, 36 mg/L sodium pyruvate, without phenol red (Life Technologies) with 1.14 mM Fluo-4 AM (Molecular Probes) and 0.25 M probenecid (Sigma) for 1 to 3 hours in the dark at room temperature. The dye solution was aspirated and the cells were washed twice with DPBS using the EL-450× cell washer (BioTech). After the last wash, 100 μL of DPBS was added to each well. Cell plates were then transferred to the FLIPR unit (Molecular Probes). Compound additions were 50 μL in duplicate of 4× final concentration in DPBS containing 0.1% BSA and 4% DMSO. Fluorescence emissions from 96 wells were measured simultaneously at excitation and emission wavelength of 488 and 520 nm, respectively for 3 minutes in 1-second intervals for the first minute and 5-second intervals thereafter. During this time agonist responses, if any, were recorded in the absence of ghrelin. Next, 50 μL in duplicate of 4× concentrated ghrelin (Bachem) solution in DPBS containing 0.1% BSA and 4% DMSO were delivered within 1 second by an integrated 96-well pipettor to a final concentration of 1 nM. Fluorescence emissions were measured for another 3 minutes as above. During this time the antagonist effects of compounds on ghrelir-stimulated calcium flux were recorded and expressed as % inhibition of the maximal ghrelin response (10 nM). Sigmoidal curves were fitted by Assay Explorer (MDL) software and IC₅₀ values determined. GHS-R antagonist [D-Lys3]-GHRP-6 (H-His-D-Trp-D-Lys-Trp-D-Phe-Lys) was purchased from Bachem and used as a positive control.

For the antagonists of ghrelin receptor, the compounds of the present invention were found to inhibit the activation of ghrelin receptor with IC₅₀ in a range of about 0.001 μM to about 10 μM in the FLIPR assays. In a preferred range, the compounds inhibit the activation of ghrelin receptor with IC₅₀ in a range of about 0.001 μM to about 1.0 μM; In a more preferred range, the compounds inhibit the activation of ghrelin receptor with IC₅₀ in a range of about 0.001 μM to about 0.1 μM.

Dihydrofolate Reductase (DHFR) Inhibition Assay

Human DHFR inhibition was assayed colorimetrically by following the nonenzymatic reduction of MTS (3-[4, 5-dimethylthiazol-2-yl]-5-(3-carboxymethoxyphenyl]-2-[4-sulfophenyl-2-H-tetrazolium, inner salt), by tetrahydrofolate, to a soluble formazan. The final assay mix (total volume of 200 μL) included potassium phosphate buffer (66 mM, pH 7.0), potassium chloride (150 mM), EDTA (1.2 mM), 2-mercaptoethanol (1 mM), NADPH (40 μM), MTS (0.025 mg/μL), dihydrofolate (30 μM), BSA (0.1 mg/mL), 1% DMSO, and 0.47 μg/mL human DHFR (Sigma, St. Louis, Mo.). A reagent mix, including the assay buffer (EDTA+KPO₄⁺KCl), 2-mercaptoethanol, NADPH, and MTS was combined, protected from light, kept on ice, and added to the test plate via a Titertek Multidrop 384. DHFR was added using a Beckman Coulter Multimek 96. The reaction was initiated with the addition of the final reagent, dihydrofolate (FAH₂), using a Multimek 96. A chiller block was used on a Multimek in a darkened room in order to prevent enzyme and substrate degradation. The test plate was then immediately transferred to a Molecular Devices Spectramax Plus 384 and read kinetically at 490 um over 2 minutes. The IC₅₀ is determined based upon the amount of drug that inhibits the rate by 50% of the control without drug. Methotrexate (Sigma, St. Louis, Mo.) was used as the positive drug control in this study.

TABLE 1
Functional Activity of Representative Ghrelin Antagonists
and Their activity against Human Dihydrofolate Reductase (hDHFR)

| | h-GHS-R | Percentage of | Percentage of |
|---|---|---|---|
| | FLIPR | Inhibition of | Inhibition of |
| Compound | IC₅₀ (μM) | hDHFR at 1.0 μM | hDHFR at 10.0 μM |
| A | 0.023 | 54.6 | 103.9 |
| B | 0.003 | 82.1 | 92.8 |
| C | 0.015 | 99.7 | 98.5 |
| D | 0.037 | 99.51 | 99.66 |
| E | 0.059 | 95.6 | 100.0 |
| F | 0.010 | 101 | 108 |
| G | 0.014 | 90.8 | 98.8 |
| H | 0.016 | 100 | 99.6 |
| I | 0.014 | 65.7 | 102 |
| J | 0.180 | 70.5 | 95.5 |

The results shown in Table 1-demonstrate that compounds A, B, C, D, E, F, G, H, I and J are all both potent ghrelin receptor antagonists and potent dihydrofolate reductase inhibitors. Moreover, the compounds of the present invention were found to antagonize the function of ghrelin in a range of about 0.001 μM to about 0.1 μM and inhibiting dihydrofolate reductase in a range of about 0.0001 μM to about 0.1 μM.

### Synthetic Methods

Abbreviations which have been used in the descriptions of the scheme and the examples that follow are: BBr₃ for boron tribromide; m-CPBA for meta-chloroperoxy-benzoic acid; DMF for N,N-dimethylformamide; DMSO for dimethylsulfoxide; DEAD for diethyl azodicarboxylate; EDAC for 1-(3-dimethylaminopropyl)-3-ethylcarbodiimide hydrochloride; HATU for O-(7-azabenzotriazol-1-yl)-N,N,N′N′-tetramethyluronium hexafluorophosphate; HOBT for 1-hydroxybenzotriazole hydrate; NMP for N-methylpyrrolidinone; NCS for N-chlorosuccinimide; MeONa for sodium methoxide; MeOH for methanol; MTBE for methyl tert butyl ether; THF for tetrahydrofuran; TFA for trifluoroacetic acid; TMSCHN₂ for trimethylsilyldiazomethane; TBAF for tetra butylammonium fluoride; Pd(dppf)Cl₂ for (diphenylphospino)ferrocenyl palladium chloride; Ph₃P for triphenylphosphine; Pr₂Net for diisopropyl ethylamine; and TBTU for (benzotriazol-1-yloxy)-dimethylamino-methylene)-dimethyl-ammonium tetrafluoroborate.

The compounds and processes of the present invention will be better understood in connection with the following synthetic schemes which illustrate the methods by which the compounds of the invention may be prepared.

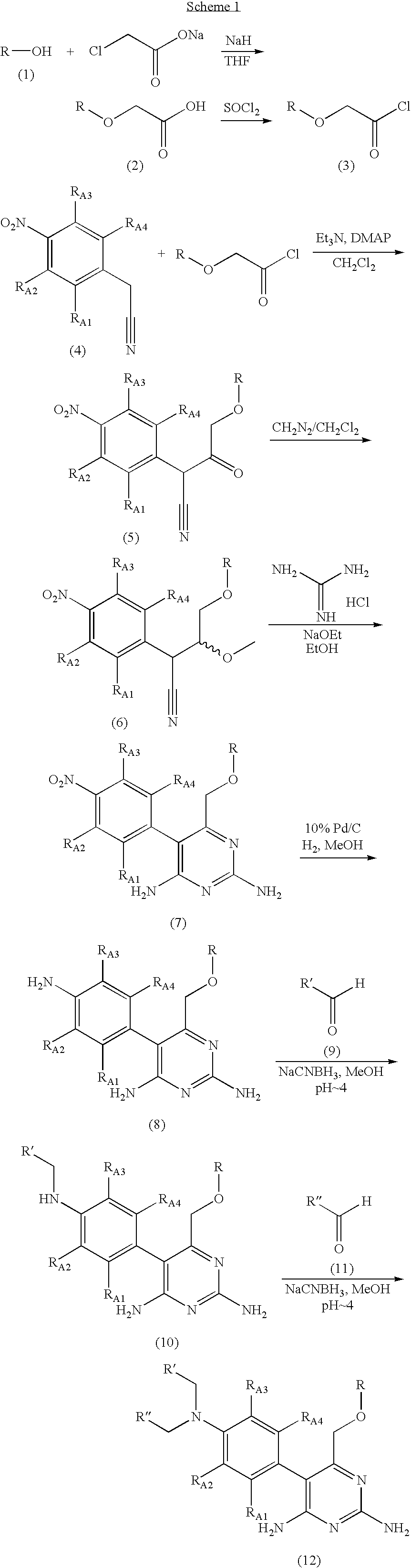

Compounds of the present invention of general formula (10), (11), (12), and (13), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 1. Phenols or alcohols of general formula (1) can be treated with sodium chloroacetate to provide acids of general formula (2). Acids of general formula (2) can be treated with thionyl chloride to provide acid chlorides of general formula (3). Acid chlorides of general formula (3) can be treated with cyano compounds of general formula (4) to provide ketones of general formula (5). Ketones of general formula (5) can be treated with diazomethane followed by treatment with guanidine to provide nitrophenylpyrimidines of general formula (7). Nitrophenylpyrimidines of general formula (7) can be reduced under conditions well known to those of skill in the art including, but not limited to, a palladium catalyst under about varying atmosphere of hydrogen to provide aminophenylpyrimidines of general formula (8). Aminophenylpyrimidines of general formula (8) can be treated with aldehydes of general formula (9) (or ketones) under reductive amination conditions to provide secondary-aminophenylpyrimidines of general formula (10). Aminophenylpyrimidines of general formula (10) can be then treated with aldehydes of general formula (11) (or ketones) under reductive amination conditions to provide tertiary-aminophenylpyrimidines of general formula (12).

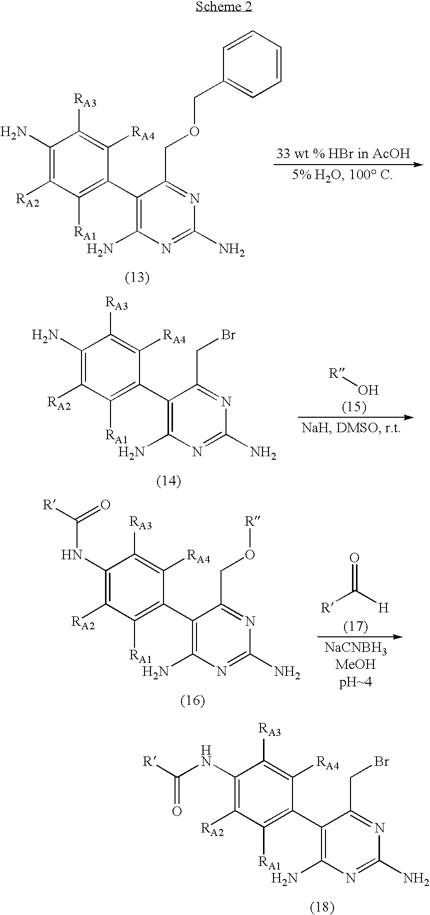

Compounds of the present invention of general formula (17), can be prepared as described in Scheme 2. Aminophenylpvrimidnes of general formula (13), prepared as described in Scheme 1, can be treated with 33 wt % HBr in AcOH at 100° C. to provide diaminopyrimidine methylbromide of general formula (14). The bromide can then be subject to standard Williamson ether synthesis to provide either compounds of general formula (16). Analogouly, other nucleophiles, such as amines, mercaptans, heterocycles, heteroaryls, can also be used to displace the bromide. Aminophenylpyrimidines of general formula (16) can be then treated with aldehydes of general formula (17) (or ketones) under reductive amination conditions to provide secondary-aminophenylpyrimidines of general formula (18).

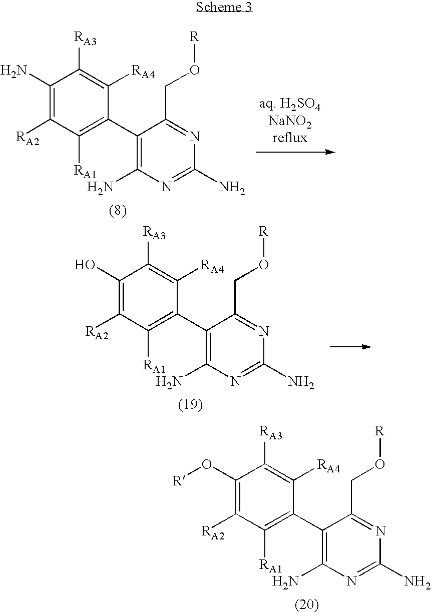

Compounds of the present invention of general formula (19) and (20), wherein R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ is hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{A1}, R_{A2}, R_{A3}, R_{A4}, R_{E}, and R_{F} are as defined in formula (I), can be prepared as described in Scheme 3. Compounds of general formula (8) can be treated with sodium nitrite and aqueous acid to provide hydroxyphenylpyrimidines of general formula (19). Hydroxyphenylpyrimidines of general formula (19) can be alkylated to provide compounds of general formula (20).

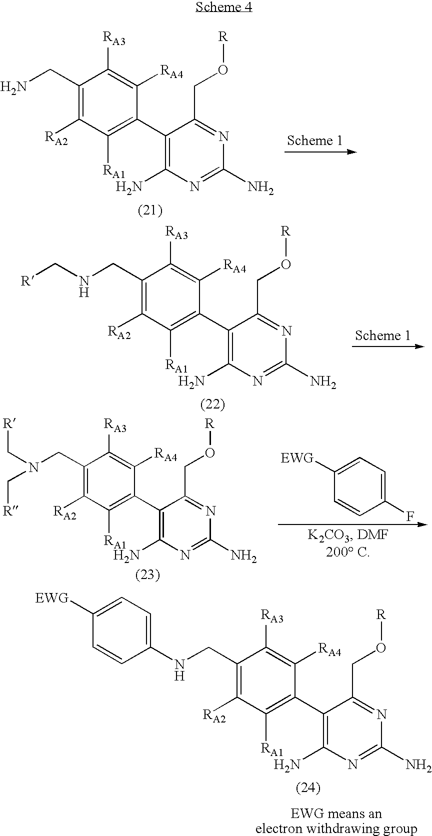

Compounds of the present invention of general formula (22), (23), (24), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 4. Aminomethylphenylpyrimidines of general formula (21), prepared as described in Examples 2 and 58 contained herein, can be alkylated/arylated as described in Scheme 1 or as described in the Examples contained herein to provide compounds of general formula (22), (23), (24).

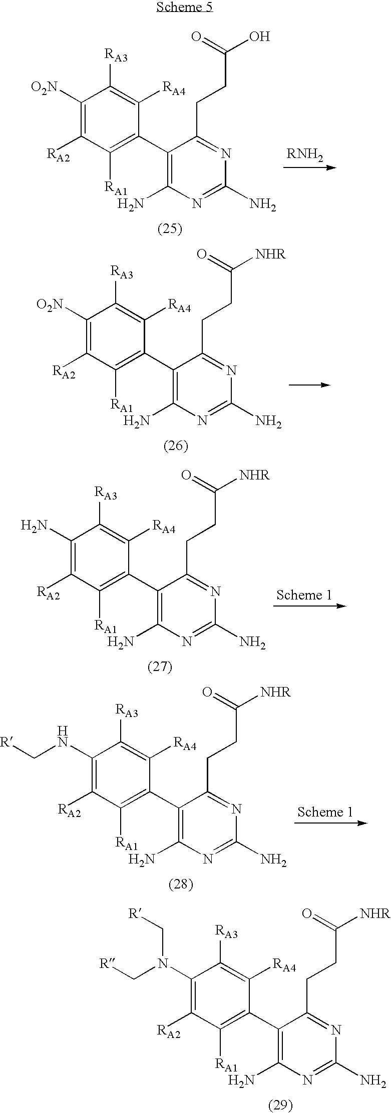

Compounds of the present invention of general formula (28), (29), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthicalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 5. Compounds of general formula (27), prepared as described in Example 57 herein, can be alkylated as described in Scheme 1 or as described in the Examples contained herein to provide compounds of general formula (28), (29).

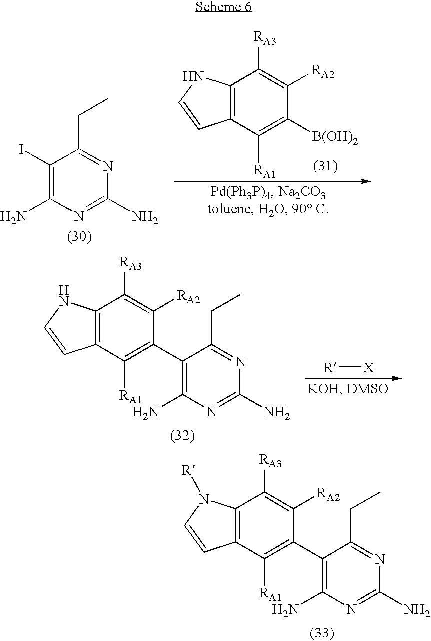

Compounds of the present invention of general formula (32) and (33), wherein R_{A1}, R_{A2}, R_{A3}, and R_{A4}, are as defined in formula (I), R′ is selected from alkoxyalkyl, alkyl, alkylthioalkyl, arylalkoxyalkyl, arylalkyl, cycloalkylalkoxyalkyl, cycloalkylalkyl, heteroarylalkoxyalkyl, heteroarylalkyl, heterocyclealkoxyalkyl, or heterocyclealkyl, can be prepared as described in Scheme 6. Compound of formula (30) when treated with compounds of formula (31) in the presence of tetrakis triphenylphosphoryl Palladium, and sodium carbonate in a mixture of toluene and water under heated conditions will provide compounds of formula (32). Compounds of formula (32) may be treated with compounds of formula R′-X wherein R′ is selected from alkoxyalkyl, alkyl, alkylthioalkyl, arylalkoxyalkyl, arylalkyl, cycloalkylalkoxyalkyl, cycloalkylalkyl, heteroarylalkoxyalkyl, heteroarylalkyl, heterocyclealkoxyalkyl, or heterocyclealkyl, and X is halo or methanesulfonyl in the presence of potassium hydroxide in DMSO will provide compounds of formula (33).

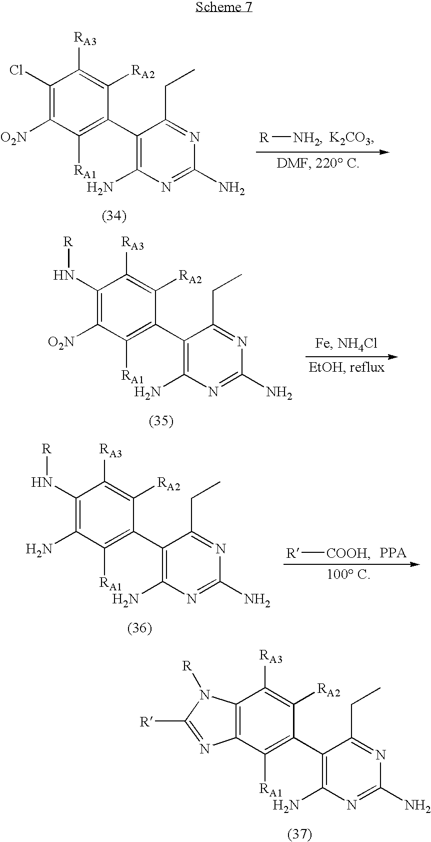

Compounds of the present invention of general formula (37), wherein R_{A1}, R_{A2}, R_{A3}, R_{A4}, and R are as defined in formula (I), and R′ is selected from alkoxyalkyl, alkyl, alkylthioalkyl, arylalkoxyalkyl, arylalkyl, cycloalkylalkoxyalkyl, cycloalkylalkyl, heteroarylalkoxyalkyl, heteroarylalkyl, heterocyclealkoxyalkyl, or heterocyclealkyl, can be prepared as described in Scheme 7. Compound of formula (34) when treated with R—NH₂, potassium carbonate in DMF under heated conditions will provide compounds of formula (35). Compounds of formula (35) when treated with iron and ammonium chloride in ethanol under heated conditions will provide compounds of formula (36). Compounds of formula (36) when treated with carboxylic acids of formula R′—C(O)—OH and poly phosphoric acid under heated conditions will provide compounds of formula (37).

The present invention will now be described in connection with certain embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments.

Compounds of the invention were named by ACD/ChemSketch version 5.01 (developed by Advanced Chemistry Development, Inc., Toronto, ON, Canada) or were given names which appeared to be consistent with ACD nomenclature.

### EXAMPLE 1

### 5-{4-[(4-chlorobenzyl)amino]phenyl}-6-ethylpyrimidine-2,4-diamine

### EXAMPLE 1A

### 2-(4-Nitro-phenyl)-3-oxo-pentanenitrile

To a solution of 8.10 g (50.0 mmol) of 4-nitrophenylacetonitrile in 100 mL of CH₂Cl₂ was added 610 mg (5 mmol) of 4-N,N-dimethylaminopyridine. The solution was cooled with an ice bath, then 8.7 mL (100 mmol) of propionyl chloride was added dropwise to avoid reflux of the solvent. After 45 minutes, the solvent was removed in vacuo, and the residue was taken up in 200 mL of 0.5 M HCl. The mixture was extracted with diethyl ether (3×50 mL), then the combined ether layers were back extracted with water (1×50 mL), brine (1×50 mL), dried over MgSO₄, filtered, and concentrated under reduced pressure to an oil.

The oil was taken up in 250 mL of methanol, and to the solution was added 200 mL of 2M NaOH. The solution was stirred for 15 minutes, then 1 L of water was added, followed by 12M HCl until precipitation was complete. The suspension was extracted with diethyl ether (2×200 mL), then the combined ether layers were back extracted with brine (1×100 mL), dried over MgSO₄, filtered, and concentrated under reduced pressure to provide the titled compound (9.3 g, 85%) as a solid. This material may be used in the next step without further purification, or maybe recrystallized from toluene to give a crystalline product.

### EXAMPLE 1B

### 6-Ethyl-5-(4-nitro-phenyl)-pyrimidine-2,4-diamine

To 1.91 g (8.75 mmol) of 2-(4-nitro-phenyl)-3-oxo-pentanenitrile from Example 1A in 20 mL of ethyl acetate was added ethereal CH₂N₂ until excess CH₂N₂ was present. The reaction was concentrated to an oil. This was taken up in 5 mL of ethanol, then treated with a premixed solution of 955 mg (10 mmol) of guanidine hydrochloride and potassium ethoxide (10 mmol) in 14 mL of ethanol. (The guanidine solution contained precipitated KCl). The reaction was stirred at reflux for 2 hours, then concentrated under reduced pressure. The residue was taken up in 20 mL of water and filtered to give a black precipitate. The precipitate was washed with 100 mL of water, recrystallized from 25 mL of ethanol. The recrystallized product was filtered, and washed with 10 mL of cold ethanol to provide the titled compound (700 mg, 27%) as green crystals.

### EXAMPLE 1C

### 5-(4-Amino-phenyl)-6-ethyl-pyrimidine-2,4-diamine

To a solution of 1.95 g (7.52 mmol) of 6-ethyl-5-(4-nitro-phenyl)-pyrimidine-2,4-diamine from Example 1B in 60 mL of glacial acetic acid was added 200 mg of 10% Pd—C. The reaction was stirred under 1 atmosphere of H₂ for 5 hours. The catalyst was filtered, and the solvent was removed under reduced pressure at 40° C. to provide a crystalline solid. The solid was dissolved in 25 mL of water, and the solution was made basic (pH=14) by the addition of 2M NaOH. The precipitate was filtered, and washed with water until the washings were pH=8. The product was dried on the filter to provide 1.55 g (90%) of light yellow crystals.

### EXAMPLE 1D

### 5-{4-[(4-Chlorobenzyl)amino]phenyl}-6-ethylpyrimidine-2,4-diamine

To a solution of 102 mg (0.445 mmol) of 5-(4-amino-phenyl)-6-ethyl-pyrimidine-2,4-diamine from Example iC in 3 mL of methanol was added a solution of 63 mg (0.45 mmol) of 4 chlorobenzaldehyde in 2 mL of methanol. The solution was stirred at ambient temperature for 10 minutes, then 1 mL of acetic acid was added, followed by 100 mg (1.59 mmol) of sodium cyanoborohydride. The solution was concentrated under reduced pressure to a volume of about 1 mL. The remainder was dissolved in 5 mL of water to whichl 10 mL of 2M NaOH was added. The formed precipitate was filtered, and washed with water until the washings were pH=8. The precipitate was recrystallized from 1 mL of ethanol to provide 46 mg (29%) of yellow crystals. ¹H NMR (300 MHz, d6-DMSO) δ 7.40 (m, 4H), 6.84 (d, 2H, J=8.5 Hz), 6.62 (d, 2H, J=8.5 Hz), 6.34 (t, 1H, J=6.1 Hz), 5.72 (s, 2H), 5.26 (bs, 2H), 4.26 (d, 2H, J=5.8 Hz), 2.10 (q, 2H, J=7.6 Hz), 0.94 (t, 3H, J=7.6 Hz); MS (ESI) m/z 354 [M+H]⁺.

### EXAMPLE 2

### 6-[(benzyloxy)methyl]-5-{4-[(4-chlorobenzyl)amino]phenyl}pyrimidine-2,4-diamine

### EXAMPLE 2A

### 4-Benzyloxy-2-(4-nitro-phenyl)-3-oxo-butyronitrile

4-Nitrophenylacetonitrile (10.0 g, 61.7 mmol), triethylamine (14.5 g, 144 mmol) and 4- (dimethylamino)pyridine (800 mg, 6.56 mmol) were dissolved in CH₂Cl₂ (150 mL). The solution was cooled to 0° C. and benzyloxyacetyl chloride (12.0 g, 64.8 mmol) was added dropwise over a 30 minutes. The reaction mixture was warmed to room temperature and stirred for 2 hours. CH₂Cl₂ was removed under reduced pressure and the mixture was dissolved in ethyl acetate (150 mL) and washed with aqueous NaHCO₃ (150 mL) and aqueous HCl (10%, 2×150 mL). The solcents were removed under reduced pressure to provide crude 4-benzyloxy-2-(4-nitro-phenyl)-3-oxo-butyronitrile (19.6 g). R_{f}=0.11 (50% ethyl acetate in hexanes)

### EXAMPLE 2B

### 6-Benzyloxymethyl-5-(4-nitro-phenyl)-pyrimidine-2,4-diamine

4-Benzyloxy-2-(4-nitro-phenyl)-3-oxo-butyronitrile (9.72 g, 31.4 mmol) was dissolved in CH₂Cl₂ (80 mL) and TMSCHN₂ (30 mL, 2M in Et₂O, 60 mmol) was added slowly. HOAc (glacial) was added dropwise until excess TMSCHN₂ was destroyed as evidenced by the cessation of N₂ evolution. The solution was concentrated under reduced pressure and the residue dissolved in 60 mL EtOH. Guanidine hydrochloride (3.605 g, 37.5 mmol) was mixed with 60 mL EtOH followed by addition of NaOEt in EtOH (14.2 mL, 37.6 mmol). After stirring the guanidine solution for 5 minutes the solution was added to the enol ether/ethanol solution resulting in a very dark, purple mixture. The reaction mixture was heated to reflux for 3 hours. The solution was concentrated under reduced pressure followed by addition of EtOAc (150 mL) and aqueous NaOH (200 mL, 0.5M). The mixture was stirred and the formed precipitate was filtered providing 6-benzyloxymethyl-5-(4-nitro-phenyl)-pyrimidine-2,4-diamine (8.78 g, 79.5%) as a light brown solid.

### EXAMPLE 2C

### 5-(4-Amino-phenyl)-6-benzyloxymethyl-pyrimidine-2,4-diamine

6-Benzyloxymethyl-5-(4-nitro-phenyl)-pyrimidine-2,4-diamine (5.00 g, 14.25 mmol) and Pd(OH)₂/C (600 mg) in MeOH (140 mL) in a heavy walled reaction vessel was charged with H₂ (60 psi) and the mixture shaken at room temperature for 14 hour. The mixture was filtered to remove the catalyst and the solution concentrated to provide 5-(4-amino-phenyl)-6-benzyloxymethyl-pyrimidine-2,4-diamine (4.34 g, 95%) as a light brown solid.

### EXAMPLE 2D

### 6-Benzyloxymethyl-5-[4-(4-chloro-benzylamino)-phenyl]-pyrimidine-2,4-diamine

5-(4-Amino-phenyl)-6-benzyloxymethyl-pyrimidine-2,4-diamine (1.62 g, 5.05 mmol) was dissolved in MeOH/NaOAc/HOAc (80 mL, 1M, pH 4). 4-Chlorobenzaldehyde (851 mg, 6.06 mmol) was added and the mixture stirred for 15 minutes. NaBH₃CN (375 mg, 6.06) was then added and the reaction mixture was stirred for 16 hours at 25° C. EtOAc (180 mL) was added and the mixture was washed with HCl (10%, 75 mL), NaOH (2M, 2×100 mL), and brine (100 mL). The crude material was purified by silica gel chromatography (EtOAc to 10% MeOH in EtOAc gradient) providing 6-benzyloxymethyl-5-[4-(4-chloro-benzylamino)-phenyl]-pyrimidine-2,4-diamine (1.56 g, 69.5%). ¹H NMR (300 MHz, DMSO-d₆) δ 3.94 (s, 2H), 4.28 (d, J=5.8 Hz, 2H), 4.32 (s, 2H), 5.49 (s, 2H), 5.87 (s, 2H), 6.37 (t, J=5.9 Hz, 1 H), 6.60 (d, J=8.5 Hz, 2H), 6.89 (d, J=8.5 Hz, 2H), 7.17 (m, 2H), 7.27 (m, 3H), and 7.40 (m, 4H). MS (ESI) positive ion 446 (M+H)⁺; negative ion 444 (M−H)⁻.

### EXAMPLE 3

### 5-{4-[(4-chlorobenzyl)amino]phenyl}-6-(methoxymethyl)pyrimidine-2,4-diamine

### EXAMPLE 3A

### [2,6-Diamino-5-(4-amino-phenyl)-pyrimidin-4-yl]-methanol

6-Benzyloxymethyl-5-(4-nitro-phenyl)-pyrimidine-2,4-diamine (550 mg, 1.57 mmol) from example 2B and Pd(OH)₂/C (550 mg) were mixed in MeOH (10 mL) under an atmosphere of nitrogen. 12 M HCl (0.75 mL, 9.0 mmol) was added to the mixture in a heavy walled vessel which was then charged with H₂ (60 psi). The mixture was shaken for 2 hours at room temperature. The catalyst was filtered, the reaction mixture washed with NaOH (2M, 50 mL), extracted in EtOAC (150 mL), and the solvent removed under reduced pressure to provide [2,6-diamino-5-(4-amino-phenyl)-pyrimidin-4-yl]-methanol (360 mg, 99%).

### EXAMPLE 3B

### {2,6-Diamino-5-[4-(4-chloro-benzylamino)-phenyl]-pyrimidin-4-yl}-methanol

[2,6-Diamino-5-(4-amino-phenyl)-pyrimidin-4-yl]-methanol from Example 3A (360 mg, 1.56 mmol) and 4-chlorobenzaldehyde (265 mg, 1.88 mmol) were dissolved in MeOH/HOAc/NaOAc (1M, 2 mL) buffer and stirred for 5 minutes. The pH was adjusted to 4, NaBH₃CN (117 mg, 1.88 mmol) was added and the mixture was for stirred 16 hours at room temperature. The mixture was concentrated under reduced pressure and purified on silica gel (5% MEOH in EtOAc) providing {2,6-diamino-5-[4-(4-chloro-benzylamino)-phenyl]-pyrimidin-4-yl}-methanol (393 mg, 71.0%) as a white solid.

### EXAMPLE 3C

### 5-{4-[(4-Chlorobenzyl)amino]phenyl}-6-(methoxymethyl)pyrimidine-2,4-diamine

{2,6-Diamino-5-[4-(4-chloro-benzylamino)-phenyl]-pyrimidin-4-yl}-methanol from Example 3B (30 mg, 0.085 mmol) was dissolved in MeOH (0.3 mL). NaH (5.0 mg, 60% dispersion in mineral oil, 0.13 mmol) was added and stirred until H₂ evolution ceased. Mel (12 mg, 0.085 mmol) was added and the mixture was stirred 16 hours. Purification was performed by reverse phase HPLC (5-100% CH₃CN in aq. NH₄Oac) providing 5-[4-(4-chloro-benzylamino)-phenyl]-6-methoxymethyl-pyrimidine-2,4-diamine (12 mg, 38 %) as an off-white solid. ¹H NMR (300 MHz, DMSO-d₆) δ 2.21 (d, J=2.03 Hz, 3H), 3.10 (s, 3H), 3.82 (s, 2H), 4.27 (d, J=5.76 Hz, 2H), 5.47 (s, 2H), 5.86 (s, 2H), 6.36 (t, J=5.93 Hz, 1H), 6.61 (d, J=8.48 Hz, 2H), 6.87 (d, J=8.48 Hz, 2H), and 7.40 (m, 4H). MS (ESI) positive ion 370 (M+H)⁺; negative ion 368 (M−H)⁻.

### EXAMPLE 4

### 5-{4-[(4-chlorobenzyl)amino]phenyl}-6-{[(2-fluoro-3-methylbenzyl)oxy]methyl}pyrimidine-2,4-diamine

[2,6-diamino-5-(4-amino-phenyl)-pyrimidin-4-yl]-methanol from Example 3B (36 mg, 0.10 mmol) and sodium tert-butoxide (15 mg, 0.156 mmol) in DMF (0.3 mL) were stirred for 20 minutes at room temperature followed by the addition of 2-fluoro-3-methyl benzyl bromide (18 mg, 0.09 mmol). The reaction mixture was stirred for 1.5 hours followed by the addition of 1 M HCl (0.1 mL) and MeOH (1.5 mL). The mixture was filtered and the resulting precipitate purified by reverse phase HPLC (0-70% CH₃CN in aq. NH₄OAc) to provide the title compound (12 mg, 28%) as an off white solid. ¹H NMR (300 MHz, DMSO-d₆) δ 3.96 (s, 2H), 4.27 (d, J=6.10 Hz, 2H), 4.37 (s, 2H), 5.55 (s, 2H), 5.93 (s, 2H), 6.37 (t, J=5.93 Hz, 1 H), 6.58 (d, J=8.82 Hz, 2H), 6.88 (d, J=8.48 Hz, 2H), 6.97 (t, J=7.46 Hz, 1H), 7.06 (m, 1 H), 7.17 (m, 1 H), and 7.39 (m, 4H). MS (ESI) positive ion 478 (M+1)⁺; negative ion 476 (M-1).

### EXAMPLE 5

### 5-{4-[(4-chlorobenzyl)amino]phenyl}-6-(3-phenylpropyl)pyrimidine-2,4-diamine

### EXAMPLE 5A

### 5-(4-Amino-phenyl)-6-(3-phenyl-propyl)-pyrimidine-2,4-diamine

The titled compound was prepared according to the procedure described in Example 2, substituting 4-phenyl-butyryl chloride for benzyloxyacetyl chloride used in Example 2.

### EXAMPLE 5B

### 5-{4-[(4-Chlorobenzyl)amino]phenyl}-6-(3-phenylpropyl)pyrimidine-2,4-diamine

To a stirred solution of 6-(4-amino-phenyl)-5-(3-phenyl-propyl)-pyrimidine-2,4-diamine from Example 5A (210 mg, 0.658 mmol) in MeOH (3.3 mL) was added 4-chlorobenzaldehyde (92 mg, 0.658 mmol). After 30 minutes at room temperature, the reaction was cooled to 0° C. Glacial acetic acid (0.185 mL, 3.3 mmol) was added followed by NaCNBK (45 mg, 0.724 mmol). The mixture was warmed to room temperature over 1.5 hour. The solvent was removed under reduced pressure, the residue was taken up in aqueous NaHCO₃ (10 mL) and washed with EtOAc (3×10 mL). The combined organic layers were washed with brine (10 mL), dried over MgSO₄,filtered and concentrated under reduced pressure. Recrystallization from EtOH resulted in a pale yellow solid (145 mg, 50%). ¹H NMR (300 MHz, DMSO-d₆) δ 7.31-7.44 (m, 5H), 7.09-7.21 (m, 3H), 7.01 (d, J=6.78 Hz, 2H), 6.82 (d, J=8.48 Hz, 2H), 6.61 (d, J=8.48, 2 H), 6.34 (t, J=5.93, 1H), 5.72 (s, 2H), 5.26 (s, 2H), 4.27 (d, J=6.10 Hz, 2H), 2.40 (t, 7.63, 2H), 2.11 (t, J=7.5 Hz, 2H), 1.72-1.77 (m, 2H). MS (ESI) positive ion 446 (M+H)⁺; negative ion 444 (M−H)⁻.

### EXAMPLE 6

### 4-({[4-(2,4-diamino-6-ethylpyrimidin-5-yl)phenyl]amino}methyl)benzonitrile

Synthesis was performed using a PE Biosystems (Applied Biosystems) Solaris 530 organic synthesizer. 4-Cyanobenzyl alcohol (0.6 mmol) was dissolved in 3 mL DMA then transferred to a 4 mL vial containing 0.5 mmol of Dess-Martin Reagent. The vial was shaken to ensure mixing then used directly. A round bottom flask was charged with 3 equivalents of MP-BH₃CN. The block was placed on the Solaris 530 and 1 equivalent of 5-(4-amino-phenyl)-6-ethyl-pyrimidine-2,4-diamine from Example 1C (dissolved in 1:1 MeOH/CH₂Cl₂) was added to the round bottom flask. The oxidized 4-cyanobenzyl alcohol was then added (2 eq) followed by 3 equivalents of a solution of HOAc in 1:1 MeOH/CH₂Cl₂. The block was then heated to 55C overnight. The mixture was transferred with MeOH to a 20 mL vial containing scavenger resins PS-TsNHNH₂ and MP-Carbonate (3 eq each). The resins were filtered and the product concentrated. Purification by Reverse Phase HPLC to provided the titled compound. ¹H NMR (500 MHz, DMSO-d₆) δ ppm 0.94 (t, J=7.49 Hz, 3H), 2.11 (q, J=7.59 Hz, 2H), 4.38 (d, J=5.93 Hz, 2 H), 5.32 (s, 2H), 5.77 (s, 2H), 6.44 (t, J=5.93 Hz, 1H), 6.61 (d, J=8.42 Hz, 2 H), 6.85 (d, J=8.42 Hz, 2H), 7.58 (d, J=8.11 Hz, 2H), 7.80 (d, J=8.11 Hz, 2H);MS (ESI) positive ion 345(M+H)⁺.

### EXAMPLE 7

### 5-{4-[(3,4-dichlorobenzyl)amino]phenyl}-6-ethylpyrimidine-2,4-diamine

The titled compound was prepared according to the procedure described in Example 6, substituting 3,4-dichlorobenzyl alcohol for 4-cyanobenzyl alcohol used in Example 6. ¹H NMR (500 MHz, DMSO-d₆) δ ppm 0.97 (t, J=7.64 Hz, 3H), 2.16 (q, J=7.69 Hz, 2H), 4.30 (d, J=5.93 Hz, 2H), 6.46 (m, 1 H), 6.64 (d, J=8.42 Hz, 2 H), 6.89 (d, J=8.42 Hz, 2 H), 7.39 (dd, J=8.26, 1.72 Hz, 1 H), 7.62 (m, 2 H); MS (ESI) positive ion 390(M+H)⁺.

### EXAMPLE 8

### 5-{4-[(4-chlorobenzyl)amino]phenyl}-6-(phenoxymethyl)pyrimidine-2,4-diamine

The title compound was synthesized according to the procedure described in Example 2, substituting phenoxyacetyl chloride for benzyloxyacetyl chloride. ¹H NMR (300 MHz, DMSO-d₆) δ 4.24 (d, J=6.10 Hz, 2 H), 4.45 (s, 2 H), 5.58 (s, 2 H), 5.93 (s, 2 H), 6.35 (t, J=5.93 Hz, 1 H), 6.58 (d, J=8.48 Hz, 2 H), 6.76 (m, 2 H), 6.87 (m, 1 H), 6.92 (d, J=8.48 Hz, 2 H), 7.19 (m, 2 H), and 7.37 (m, 4 H). MS (ESI) positive ion 432 (M+H)⁺; negative ion 430 (M−H)⁻.

### EXAMPLE 9

### 5-{4-[(4-chlorobenzyl)amino]phenyl}-6-propylpyrimidine-2,4-diamine

### EXAMPLE 9A

### 5-(4-Amino-phenyl)-6-propyl-pyrimidine-2.4-diamine

The titled compound was prepared according to the procedure described in Example 2, substituting butyryl chloride for benzyloxyacetyl chloride used in Example 2.

### EXAMPLE 9B

### 5-{4-[(4-chlorobenzyl)amino]phenyl}-6-propylpyrimidine-2,4-diamine

To a stirred solution of 6-(4-amino-phenyl)-5-propyl-pyrimidine-2,4-diamine from Example 9A (100 mg, 0.411 mmol) in MeOH (2.0 mL) was added 4-chlorobenzaldehyde (57 mg, 0.411 mmol). The mixture was stirred for 20 minutes at room temperature then cooled to 0° C. Glacial acetic acid (0.100 mL, 1.7 mmol) was added followed by NaCNBH₃ (28 mg, 0.452 mmol). The mixture was warmed to room temperature over 1 hour and the solvent removed under reduced pressure. The residue was taken up in aqueous NaHCO₃ (5 mL) and washed with EtOAc (2×5 mL). The combined organic layers were washed with brine (5 mL), dried over MgSO₄, filtered and concentrated under reduced pressure. The residue was triturated from 1 mL of 10:1:0.1 CH₂Cl₂:MeOH:NH₄OH and filtered to provide a white solid (77 mg, 52%).¹H NMR (300 MHz, DMSO-d₆) δ 7.40-7.44 (m, 4H), 6.83 (d, J=8.48 Hz, 2H), 6.62 (d, J=8.48 Hz, 2H), 6.34 (t, J=6.10 Hz, 1H), 5.83 (s, 2H), 5.40 (s, 2H), 4.26 (d, J=5.76 Hz, 2H), 2.062.11 (m, 2H), 1.40-1.47 (m, 2H), 0.716 (t, J=7.29 Hz, 3H). MS(ESI) positive ion 368 (M+H)⁺; negative ion 366 (M−H)⁻.

### EXAMPLE 10

### 5-{4-[(4-chlorobenzyl)amino]phenyl}-6-{[(3-methylbenzyl)oxy]methyl}pyrimidine-2,4-diamine

To 1-(bromomethyl)-3-methylbenzene (0.06 mmol) in 0.31 mL of DMF was added a solution of {2,6-diamino-5-[4-(4-chloro-benzylamino)-phenyl]-pyrimidin-4-yl}-methanol from Example 3B (0.07 mmol) and NaOtBu (0.105 mmol) in 0.6 mL DMF. The mixture was heated to 55° C. overnight then concentrated under reduced pressure to dryness. Purification by Reverse Phase Chromatography provided the titled compound. ¹H NMR (500 MHz, DMS-d₆) δ ppm 2.27 (m, 3 H), 3.31 (m, 4 H), 3.96 (m, 2 H), 4.27 (m, 4 H), 6.35 (t, J=6.08 Hz, 1 H), 660 (d, J=8.42 Hz, 2 H), 6.89 (d, J=8.73 Hz, 2 H), 6.96 (d, J=7.49 Hz, 1 H), 6.99 (s, 1 H), 7.04 (d, J=7.49 Hz, 1 H), 7.14 (t, J=7.64 Hz, 1 H), 7.39 (m, 4 H); MS (ESI) positive ion 460(M+H)⁺.

### EXAMPLE 11

### 5-{4-[(4-chlorobenzyl)amino]phenyl}-6-{[(2-methoxybenzyl)oxy]methyl}pyrimidine-2,4-diamine

The titled compound was prepared according to the procedure described in Example 10, substituting 2-methoxybenzyl bromide for 1-(bromomethyl)-3-methylbenzene used in Example 10. ¹H NMR (500 MHz, DMSO-d₆) δ ppm 1.93 (m, 3 H), 3.73 (s, 2 H), 3.96 (s, 2 H), 4.27 (d, J=5.93 Hz, 2 H), 4.33 (s, 2 H), 5.85 (s, 2 H), 6.34 (t, J=5.93 Hz, 1 H), 6.59 (d, J=8.42 Hz, 2 H), 6.83 (t, J=7.49 Hz, 1 H), 6.91 (m, 3 H), 7.13 (d, J=7.48 Hz, 1 H), 7.22 (m, 1 H), 7.40 (q, J=8.52 Hz, 4 H); MS (ESI) positive ion 476(M+H)⁺.

### EXAMPLE 12

### 5-{4-[(4-chlorobenzyl)amino]phenyl}-6-{[(3-methoxybenzyl)oxy]methyl}pyrimidine-2,4-diamine

The titled compound was prepared according to the procedure described in Example 10, substituting 3-methoxybenzyl bromide for 1-(bromomethyl)-3-methylbenzene used in Example 10. ¹H NMR (500 MHz, DMSO-d₆) δ ppm 3.72 (d, J=6.24 Hz, 3 H), 3.95 (s, 2 H), 4.27 (d, J=5.93 Hz, 2 H), 4.30 (s, 2 H), 5.52 (m, 1 H), 5.86 (s, 2 H), 6.34 (t, J=6.08 Hz, 1 H), 660 (d, J=8.42 Hz, 2 H), 6.78 (m, 3 H), 6.89 (d, J=8.73 Hz, 2 H), 7.17 (t, J=7.80 Hz, 1 Hz), 7.39 (m, 4 H); MS (ESI) positive ion 476(M+H)⁺.

### EXAMPLE 13

### 5-{4-[(4-chlorobenzyl)amino]phenyl}-6-{[(4-methoxybenzyl)oxy]methyl}pyrimidine-2,4-diamine

The titled compound was prepared according to the procedure described in Example 10, substituting 4-methoxybenzyl bromide for 1-(bromomethyl)-3-methylbenzene used in Example 10. ¹H NMR (500 MHz, DMSO-d₆) δ ppm 3.73 (s, 3 H), 3.90 (s, 2 H), 4.23 (s, 2 H), 4.28 (d, J=5.93 Hz, 2 H), 5.47 (m, 2 H), 5.85 (s, 2 H), 6.34 (t, J=6.08 Hz, 1H), 6.60 (d, J=8.42 Hz, 2 H), 6.86 (dd, J=28.23, 8.58 Hz, 4 H), 7.10 (d, J=8.73 Hz, 2 H), 7.40 (m, 4 H); MS (ESI) positive ion 476(M+H)⁺.

### EXAMPLE 14

### 5-{4-[(4-chlorobenzyl)amino]phenyl}-6-{[(2-fluorobenzyl)oxy]methyl}pyrimidine-2,4-diamine

The titled compound was prepared according to the procedure described in Example 10, substituting 2-fluorobenzyl bromide for 1-(bromomethyl)-3-methylbenzene used in Example 10. ¹H NMR (500 MHz, DMSO-d₆) δ ppm 3.96 (s, 2 H), 4.27 (d, J=5.61 Hz, 2 H), 4.39 (s, 2 H), 5.86 (s, 2H), 6.34 (t, J=5.93 Hz, 1 H), 6.59 (d, J=8.73 Hz, 2 H), 6.89 (d, J=8.42 Hz, 2 H), 7.11 (m, 2 H), 7.29 (m, 2 H), 7.40 (m, 4 H); MS (ESI) positive ion 464(M+H)⁺.

### EXAMPLE 15

### 5-{4-[(4-chlorobenzyl)amino]phenyl}-6-{[(4-fluorobenzyl)oxy]methyl}pyrimidine-2,4-diamine

The titled compound was prepared according to the procedure described in Example 10, substituting 4-fluorobenzyl bromide for 1-(bromomethyl)-3-methylbenzene used in Example 10. ¹H NMR-(500 MHz, DMSO-d₆) δ ppm 3.93 (s, 2 H), 4.27 (d, J=5.93 Hz, 2 H), 4.30 (s, 2 H), 5.85 (s, 2 H), 6.35 (t, J=5.93 Hz, 1 H), 6.60 (d, J=8.42 Hz, 2 H), 6.88 (d, J=8.42 Hz, 2 H), 7.07 (t, J=8.89 Hz, 2 H), 7.20 (dd, J=8.42, 5.61 Hz, 2 H), 7.39 (m, 4 H); MS(ESI) positive ion 464(M+H)⁺.

### EXAMPLE 16

### 5-{4-[(4-chlorobenzyl)amino]phenyl}-6-{[(2-chlorobenzyl)oxy]methyl}pyrimidine-2,4-diamine

The titled compound was prepared according to the procedure described in Example 10, substituting 2-chlorobenzyl bromide for 1-(bromomethyl)-3-methylbenzene used in Example 10. ¹H NMR (500 MHz, DMSO-d₆) δ ppm 4.01 (s, 2 H), 4.27 (d, J=5.93 Hz, 2 H), 4.41 (s, 2 H), 5.51 (m, 2 H), 5.87 (s, 2 H), 6.35 (t, J=5.93 Hz, 1 H), 6.59 (d, J=8.42 Hz, 2 H), 6.90 (d, J=8.42 Hz, 2 H), 7.27 (m, 3 H), 7.39 (m, 5 H); MS(ESI) positive ion 481(M+H)⁺.

### EXAMPLE 17

### 5-{4-[(4-chlorobenzyl)amino]phenyl}-6-{[(4-chlorobenzyl)oxy]methyl}pyrimidine-2,4-diamine

The titled compound was prepared according to the procedure described in Example 10, substituting 4-chlorobenzyl bromide for 1-(bromomethyl)-3-methylbenzene used in Example 10. ¹H NMR (500 MHz, DMSO-d₆) δ ppm 3.94 (s, 2 H), 4.27 (d, J=5.93 Hz, 2 H), 4.32 (s, 2 H), 5.85 (s, 2 H), 6.35 (t, J=6.08 Hz, 1 H), 6.60 (d, J=8.73 Hz, 2 H), 6.88 (d, J=8.42 Hz, 2 H), 7.18 (d, J=8.42 Hz, 2 H), 7.32 (d, J=8.42 Hz, 2 H), 7.40 (m, 4 H); MS(ESI) positive ion 481(M+H⁺.

### EXAMPLE 18

### 6-{[(2-bromobenzyl)oxy]methyl}-5-{4-[(4-chlorobenzyl)amino]phenyl}pyrimidine-2,4-diamine

The titled compound was prepared according to the procedure described in Example 10, substituting 2-bromobenzyl bromide for 1-(bromomethyl)-3-methylbenzene used in Example 10. ¹H NMR (500 MHz, DMSO-d₆) δ ppm 4.02 (s, 2 H), 4.27 (d, J=5.93 Hz, 2 H), 4.38 (s, 2 H), 5.89 (s, 2 H), 6.35 (t, J=6.08 Hz, 1 H), 6.59 (d, J=8.73 Hz, 2 H), 6.90 (d, J=8.42 Hz, 2 H), 7.20 (m, 1 H), 7.28 (m, 2 H), 7.39 (m, 4 H), 7.55 (d, J=7.49 Hz, 1 H); MS(ESI) positive ion 525(M+H)⁺.

### EXAMPLE 19

### 5-{4-[(4-chlorobenzyl)amino]phenyl}-6-({[3-(trifluoromethyl)benzyl]oxy}methyl)pyrimidine-2,4-diamine

The titled compound was prepared according to the procedure described in Example 10, substituting 3-1′,1′,1′-trifluoromethylbenzyl bromide for 1-(bromomethyl)-3-methylbenzene used in Example 10. ¹H NMR (500 MHz, DMSO-d₆) δ ppm 4.00 (s, 2 H), 4.26 (d, J=5.93 Hz, 2 H), 4.43 (s, 2 H), 5.89 (m, 2 H), 6.35 (t, J=6.08 Hz, 1 H), 6.59 (d, J=8.42 Hz, 2 H), 6.88 (d, J=8.42 Hz, 2 H), 7.39 (m, 4 H), 7.49 (m, 2 H), 7.58 (m, 2H); MS(ESI) positive ion 514(M+H)⁺.

### EXAMPLE 20

### 5-{4-[(4-chlorobenzyl)amino]phenyl}-6-({[4-(methylthio)benzyl]oxy]methyl)pyrimidine-2,4-diamine

The titled compound was prepared according to the procedure described in Example 10, substituting 1-bromomethyl-4-methylsulfanyl-benzene for 1-(bromomethyl)-3-methylbenzene used in Example 10. ¹H NMR (500 MHz, DMSO-d₆) δ ppm 3.92 (s, 2 H), 4.28 (m, 4 H), 5.43 (m, 2 H), 5.84 (s, 2 H), 6.34 (t, J=5.93 Hz, 1 H), 6.60 (d, J=8.42 Hz, 2 H), 6.89 (d, J=8.42 Hz, 2 H), 7.11 (d, J=8.42 Hz, 2 H), 7.17 (m, 2 H), 7.40 (m, 5 H); MS(ESI) positive ion 492(M+H)⁺.

### EXAMPLE 21

### 5-{4-[(4-chlorobenzyl)amino]phenyl}-6-{[(2,4-dimethylbenzyl)oxy]methyl}pyrimidine-2,4-diamine

The titled compound was prepared according to the procedure described in Example 10, substituting 1-bromomethyl-2,4-dimethyl-benzene for 1-(bromomethyl)-3-methylbenzene used in Example 10. ¹H NMR (500 MHz, DMSO-d₆) δ ppm 2.12 (s, 3 H), 2.23 (s, 3 H), 3.93 (s, 2 H), 4.27 (m, 4 H), 5.45 (s, 2 H), 5.83 (s, 2 H), 6.35 (t, J=5.77 Hz, 1 H), 6.59 (d, J=8.42 Hz, 2 H), 6.88 (d, J=8.42 Hz, 3 H), 6.94 (m, 1 H), 7.00 (d, J=7.80 Hz, 1 H), 7.40 (m, 4 H); MS (ESI) positive ion 474(M+H)⁺.

### EXAMPLE 22

### 5-{4-[(4-chlorobenzyl)amino]phenyl}-6-{[(3,5-dimethylbenzyl)oxy]methyl}pyrimidine-2,4-diamine

The titled compound was prepared according to the procedure described in Example 10, substituting 1-bromomethyl-3,5-dimethyl-benzene for 1-(bromomethyl)-3-methylbenzene used in Example 10. ¹H NMR (500 MHz, DMSO-d₆) δ ppm 2.22 (s, 6H), 3.92 (s, 2H), 4.24 (s, 2 H), 4.27 (d, J=5.93 Hz, 2 H), 5.46 (s, 2 H), 5.85 (s, 2 H), 6.34 (t, J=5.93 Hz, 1 H), 6.60 (d, J=8.42 Hz, 2 H), 6.78 (s, 2 H), 6.86 (s, 1 H), 6.89 (d, J=8.73 Hz, 2 H), 7.38 (m, 4 H);MS (ESI) positive ion 474(M+H)⁺.

### EXAMPLE 23

### 5-{4-[(4-chlorobenzyl)amino]phenyl}-6-{[(2,3-dichlorobenzyl)oxy]methyl}pyrimidine-2,4-diamine

The titled compound was prepared according to the procedure described in Example 10, substituting 2,3-dichlorbenzyl bromide for 1-bromomethyl)-3-methylbenzene used in Example 10. ¹H NMR (500 MHz, DMSO-d₆) δ ppm 4.02 (s, 2 H), 4.26 (d, J=5.93 Hz, 2 H), 4.45 (s, 2 H), 5.50 (s, 2 H), 5.87 (s, 2 H), 6.35 (t, J=6.08 Hz, 1 H), 6.58 (d, J=8.42 Hz, 2 H), 6.88 (d, J=8.42 Hz, 1 H), 7.26 (m, 2 H), 7.38 (m, 5 H), 7.53 (dd, J=7.17, 2.50 Hz, 1 H); MS(ESI) positive ion 516(M+H)⁺.

### EXAMPLE 24

### 5-{4-[(4-chlorobenzyl)amino]phenyl}-6-{[(2,5-dichlorobenzyl)oxy]methyl}pyrimidine-2,4-diamine

The titled compound was prepared according to the procedure described in Example 10, substituting 2,5-dichlorbenzyl bromide for 1-(bromomethyl)-3-methylbenzene used in Example 10. ¹H NMR (500 MHz, DMSO-d₆) δ ppm 4.05 (s, 2 H), 4.26 (d, J=5.61 Hz, 2 H), 4.40 (s, 2 H), 5.53 (s, 2 H), 5.89 (s, 2 H), 6.34 (t, J=5.93 Hz, 1 H), 6.59 (d, J=8.73 Hz, 2 H), 6.88 (d, J=8.42 Hz, 2 H), 7.39 (m, 7 H); MS (ESI) positive ion 516(M+H)⁺.

### EXAMPLE 25

### 5-{4-[(1,3-benzodioxol-4-ylmethyl)amino]phenyl}-6-[(benzyloxy)methyl}pyrimidine-2,4-diamine

The titled compound was synthesized according to the procedure described in Example 2, substituting 2.3-(methylenedioxy)benzaldehyde for 4-chlorobenzaldehyde. ¹H NMR (300 MHz, DMSO-d₆) δ 3.95 (s, 2 H), 4.21 (d, J=5.76 Hz, 2 H), 4.33 (s, 2 H), 5.50 (s, 2 H), 5.88 (s, 2 H), 6.04 (s, 2 H), 6.22 (t, J=5.93 Hz, 1 H), 6.63 (d, J=8.48 Hz, 2 H), 6.81 (m, 2 H), 6.89 (m, 1 H), 6.91 (d, J=8.48 Hz, 2 H), 7.19 (m, 2 H), and 7.27 (m, 3 H). MS (ESI) positive ion 456 (M+H)⁺; negative ion 454 (M−H)⁻.

### EXAMPLE 26

### 6-[(benzyloxy)methyl]-5-{4-[(2-furylmethyl)amino]phenyl}pyrimidine-2,4-diamine

The titled compound was synthesized according to the procedure described in Example 2, substituting 2-furaldehyde for 4-chlorobenzaldehyde. ¹H NMR (300 MHz, DMSO-d₆) δ 3.96 (s, 2 H), 4.26 (d, J=6.10 Hz, 2 H), 4.34 (s, 2 H), 5.54 (s, 2 H), 5.91 (s, 2 H), 6.19 (t, J=5.93 Hz, 1 H), 6.33 (dd, J=3.22, 0.85 Hz, 1 H), 6.39 (m, 1 H), 6.69 (d, J=8.82 Hz, 2 H), 6.92 (d, J=8.48 Hz, 2 H), 7.19 (m, 2 H), 7.28 (m, 3 H), and 7.58 (dd, J=1.86, 0.85 Hz, 1 H). MS (ESI) positive ion 402 (M+H)⁺; negative ion 400 (M−H)⁻.

### EXAMPLE 27

### 6-[(benzyloxy)methyl]-5-{4-[(tetrahydrofuran-3-ylmethyl)amino]phenyl}pyrimidine-2,4-diamine

The titled compound was synthesized according to the procedure described in Example 2, substituting tetrahydrofuran-3-carboxaldehyde for 4-chlorobenzaldehyde. ¹H NMR (300 MHz, DMSO-d₆) δ 1.61 (m, 1 H), 2.01 (m, 1 H), 2.48 (m, 1 H), 3.00 (m, 2 H), 3.47 (dd, J=8.48, 5.42 Hz, 1 H), 3.64 (m, 1 H), 3.77 (m, 2 H), 3.97 (s, 2 H), 4.35 (s, 2 H), 5.55 (s, 2 H), 5.81 (t, J=5.59 Hz, 1 H), 5.90 (s, 2 H), 6.61 (d, J=8.48 Hz, 2 H), 6.91 (d, J=8.48 Hz, 2 H), 7.20 (m, 2 H), and 7.28 (m, 3 H). MS (ESI) positive ion 406 (M+H)⁺; negative ion 404 (M−H)⁻.

### EXAMPLE 28

### 6-[(benzyloxy)methyl]-5-{4-[(pyridin-2-ylmethyl)amino]phenyl}pyrimidine-2,4-diamine

The title compound was synthesized according to the procedure described in Example 2, substituting 2-pyridinecarboxaldehyde for 4-chlorobenzaldehyde. ¹H NMR (300 MHz, DMSO-d₆) δ 3.94 (s, 2 H), 4.32 (s, 2 H), 4.37 (d, J=6.10 Hz, 2 H), 5.48 (s, 2 H), 5.87 (s, 2 H), 6.42 (t, J=5.93 Hz, 1 H), 6.62 (d, J=8.48 Hz, 2 H), 6.90 (d, J=8.48 Hz, 2 H), 7.18 (m, 2 H), 7.25 (m, 4 H), 7.41 (d, J=7.80 Hz, 1 H), 7.74 (td, J=7.63, 2.03 Hz, 1 H), and 8.54 (ddd, J=4.83, 1.78, 0.85 Hz, 1 H). MS (ESI) positive ion 413 (M+H)⁺; 411 (M−H)⁻.

### EXAMPLE 29

### 6-[(benzyloxy)methyl]-5-}4-[(pyridin-3-ylmethyl)amino]phenyl}pyrimidine-2,4-diamine

The title compound was synthesized according to the procedure described in Example 2, substituting 3-pyridinecarboxaldehyde for 4-chlorobenzaldehyde. ¹H NMR (300 MHz, DMSO-d₆) δ 3.94 (s, 2 H), 4.32 (d, 5.81 Hz, 2 H), 4.32 (s, 2 H), 5.50 (s, 2 H), 5.88 (s, 2 H), 6.36 (t, J=5.93 Hz, 1 H), 6.64 (d, J=8.48 Hz, 2 H), 6.91 (d, J=8.82 Hz, 2 H), 7.18 (m, 2 H), 7.26 (m, 3 H), 7.36 (ddd, J=7.80, 4.75, 0.68 Hz, 1 H), 7.79 (dt, J=7.80, 187 Hz, 1 H), 8.46 (dd, J=4.92, 1.53 Hz, 1 H), and 8.62 (d, J=2.37 Hz, 1 H). MS (ESI) positive ion 413 (M+H)⁺.

### EXAMPLE 30

### 6-[(benzyloxy)methyl]-5-{4-[(pyridin-4-ylmethyl)amino]phenyl}pyrimidine-2,4-diamine

The title compound was synthesized according to the procedure described in Example 2, substituting 4-pyridinecarboxaldehyde for 4-chlorobenzaldehyde. ¹H NMR (300 MHz, DMSO-d₆) δ 3.93 (s, 2 H), 4.32 (s, 2 H), 4.33 (d, J=7.12 Hz, 2 H), 5.49 (s, 2 H), 5.87 (s, 2 H), 6.45 (t, J=5.93 Hz, 1 H), 6.59 (d, J=8.48 Hz, 2 H), 6.90 (d, J=8.48 Hz, 2 H), 7.17 (m, 2 H), 7.26 (m, 3 H), 7.38 (d, J=5.76 Hz, 2 H), and 8.50 (m, 2 H). MS (ESI) positve ion 413 (M+H)⁺; negative ion 411 (M−H)⁻.

### EXAMPLE 31

### 6-[(benzyloxy)methyl]-5-{4-[(1 H-imidazol-2-ylmethyl)amino]phenyl}pyrimidine-2,4-diamine

The title compound was synthesized according to the procedure described in Example 2, substituting 2-imidazolcarboxaldehyde for 4-chlorobenzaldehyde. ¹H NMR (300 MHz, DMSO-d₆) δ 3.96 (s, 2 H), 4.25 (d, J=5.76 Hz, 2 H), 4.34 (s, 2 H), 5.47 (br s, 2 H), 5.88 (s, 2 H), 6.12 (t, J=5.43 Hz, 1 H), 6.70 (d, J=8.48 Hz, 2 H), 6.84 (br s, 1 H), 6.92 (d, J=8.48 Hz, 2 H), 7.02 (br s, 1 H), 7.20 (m, 2 H), 7.28 (m, 3 H), and 11.87 (s, 1 H); MS (ESI) positive ion 402 (M+H)⁺; negative ion 400 (M−H)⁻.

### EXAMPLE 32

### 6-[(benzyloxy)methyl]-5-{4-[(1H-imidazol-4-ylmethyl)amino]phenyl}pyrimidine-2,4-diamine

The title compound was synthesized according to the procedure described in Example 2, substituting 4(5)imidazolecarboxaldehyde for 4-chlorobenzaldehyde. ¹H NMR (300 MHz, DMSO-d₆) δ 3.96 (s, 2 H), 4.15 (d, J=3.73 Hz, 2 H), 4.34 (s, 2 H), 5.49 (s, 2 H), 5.88 (s, 2 H), 5.90 (t, J=5.34 Hz, 1 H), 6.69 (d, J=8.82 Hz, 2 H), 6.91 (d, J=8.48 Hz, 2 H), 6.97 (s, 1 H), 7.20 (m, 2 H), 7.28 (m, 3 H), and 7.57 (d, J=1.02 Hz, 1 H). MS (ESI) postive ion 402 (M+H)⁺; negative ion 400 (M−H)⁻.

### EXAMPLE 33

### 6-[(benzyloxy)methyl]-5-[4-(dimethylamino)phenyl]pyrimidine-2,4-diamine

The titled compound was synthesized according to the procedure described in Example 2, substituting excess formaldehyde for 4-chlorobenzaldehyde. ¹H NMR (300 MHz, DMSO-d₆) δ 2.93 (s, 6 H), 3.97 (s, 2 H), 4.35 (s, 2 H), 5.56 (s, 2 H), 5.93 (s, 2 H), 6.75 (d, J=8.82 Hz, 2 H), 7.02 (d, J=8.82 Hz, 2 H), 7.20 (m, 2 H), and 7.28 (m, 3 H). MS (ESI) positive ion 350 (M+H)⁺; negative ion 348 (M−H)⁻.

### EXAMPLE 34

### 6-[(benzyloxy)methyl]-5-[4-(methylamino)phenyl]pyrimidine-2,4-diamine

The titled compound was synthesized according to the procedure described in Example 2, substituting formaldehyde for 4-chlorobenzaldehyde. ¹H NMR (300 MHz, DMSO-d₆) δ 2.70 (d, J=4.75 Hz, 3 H), 3.97 (s, 2 H), 4.35 (s, 2 H), 5.51 (s, 2 H), 5.71 (q, J=5.09 Hz, 1 H), 5.88 (s, 2 H), 6.57 (d, J=8.81 Hz, 2 H), 6.93 (d, J=8.48 Hz, 2 H), 7.21 (m, 2 H), and 7.28 (m, 3 H). MS (ESI) positive ion 336 (M+H)⁺.

### EXAMPLE 35

### 6-[(benzyloxy)methyl]-5-[4-(ethylamino)phenyl]pyrimidine-2,4-diamine

The titled compound was synthesized according to the procedure described in Example 2, substituting acetaldehyde for 4-chlorobenzaldehyde. ¹H NMR (300 MHz, DMSO-d₆) δ 1.18 (t, J=7.12 Hz, 3 H), 3.05 (m, 2 H), 3.97 (s, 2 H), 4.35 (s, 2 H), 5.54 (s, 2 H), 5.61 (m, 1 H), 5.90 (s, 2 H), 6.58 (d, J=8.48 Hz, 2 H), 6.91 (d, J=8.48 Hz, 2 H), 7.21 (m, 2 H), and 7.28 (m, 3 H). MS (ESI) 350 positive ion (M+H)⁺.

### EXAMPLE 36

### 6-[(benzyloxy)methyl]-5-[4-(propylamino)phenyl]pyrimidine-2,4-diamine

The titled compound was synthesized according to the procedure described in Example 2, substituting propionaldehyde for 4-chlorobenzaldehyde. ¹H NMR (300 MHz, DMSO-d₆) δ 0.96 (t, J=7.29 Hz, 3 H), 1.58 (sextet, J=7.12 Hz, 2 H), 2.98 (m, 2 H), 3.97 (s, 2 H), 4.35 (s, 2 H), 5.49 (s, 2 H), 5.66 (t, J=5.43 Hz, 1 H), 5.86 (s, 2 H), 6.59 (d, J=8.48 Hz, 2 H), 6.90 (d, J=8.48 Hz, 2 H), 7.20 (m, 2 H), and 7.28 (m, 3 H). MS (ESI) positive ion 364 (M+H)⁺; negative ion 362 (M−H)⁻.

### EXAMPLE 37

### 6-[(benzyloxy)methyl]-5-[4-(isobutylamino)phenyl]pyrimidine-2,4-diamine

The title compound was synthesized according to the procedure described in Example 2, substituting isobutyraldehyde for 4-chlorobenzaldehyde. ¹H NMR (300 MHz, DMSO-d₆) δ 0.95 (d, J=6.44 Hz, 6 H), 1.85 (m, 1 H), 2.83 (t, J=6.27 Hz, 2 H), 3.97 (s, 2 H), 4.35 (s, 2 H), 5.50 (s, 2 H), 5.71 (t, J=5.76 Hz, 1 H), 5.86 (s, 2 H), 6.59 (d, J=8.48 Hz, 2 H), 6.90 (d, J=8.48 Hz, 2 H), 7.20 (m, 2 H), and 7.27 (m, 3 H). MS (ESI) positive ion 378 (M+H)⁺.

### EXAMPLE 38

### 6-[(benzyloxy)methyl]-5-[4-(neopentylamino)phenyl]pyrimidine-2,4-diamine

The titled compound was synthesized according to the procedure described in Example 2, substituting trimethylacetaldehyde for 4-chlorobenzaldehyde. ¹H NMR (300 MHz, DMSO-d₆) δ 0.97 (s, 9 H), 2.83 (d, J=5.76 Hz, 2 H), 4.03 (s, 2 H), 4.38 (s, 2 H), 5.57 (t, J=5.76 Hz, 1 H), 6.04 (s, 2 H), 6.29 (s, 2 H), 6.68 (d, J=8.48 Hz, 2 H), 6.89 (d, J=8.48 Hz, 2 H), 7.22 (m, 2 H), and 7.30 (m, 3 H). MS (ESI) positive ion 392 (M+H)⁺; negative ion 390 (M−H)⁻.

### EXAMPLE 39

### 6-[(benzyloxy)methyl]-5-{4-[(cyclopropylmethyl)amino]phenyl}pyrimidine-2,4-diamine

The title compound was synthesized according to the procedure described in Example 2, substituting cycloproylcarboxaldehyde for 4-chlorobenzaldehyde. ¹H NMR (300 MHz, DMSO-d₆) δ 0.22 (ddd, J=5.85, 4.49, 4.24 Hz, 2 H), 0.48 (ddd, J=8.05, 5.85, 4.07 Hz, 2 H), 1.06 (m, 1 H), 2.90 (t, J=5.93 Hz, 2 H), 3.98 (s, 2 H), 4.35 (s, 2 H), 5.56 (s, 2 H), 5.74 (t, J=5.43 Hz, 1 H), 5.93 (s, 2 H), 6.61 (d, J=8.48 Hz, 2 H), 6.91 (d, J=8.48 Hz, 2 H), 7.21 (m, 2 H), and 7.29 (m, 3 H). MS (ESI) positive ion 376 (M+H)⁺; negative ion 374 (M−H)⁻.

### EXAMPLE 40

### 5-{4-[(4-chlorobenzyl)amino]phenyl}-6-tetrahydrofuran-2-ylpyrimidine-2,4-diamine

### EXAMPLE 40A

### 5-(4-Amino-phenyl)-6-(tetrahydro-furan-2-yl)-pyrimidine-2,4-diamine

The titled compound was prepared according to the procedure described in Example 2, substituting tetrahydrofuran-2-carbonyl chloride for benzyloxyacetyl chloride used in Example 2.

### EXAMPLE 40B

### 5-{4-[(4-Chlorobenzyl)amino]phenyl}-6-tetrahydrofuran-2-ylpyrimidine-2,4-diamine

To a stirred solution of 5-(4-amino-phenyl)-6-(tetrahydrofuran-2-yl)-pyrimidine-2,4-diamine from Example 40A (40 mg, 0.147 mmol) in MeOH (1.5 mL) was added 4-chlorobenzaldehyde (20 mg, 0.147 mmol). After mixture was stirred for 30 minutes at room temperature then cooled to 0° C. Glacial acetic acid (0.03 mL, 0.53 mmol) was added followed by NaCNBH₃ (10 mg, 0.162 mmol). The reaction warmed to room temperature over 2 hours and the solvent removed under reduced pressure. The residue was taken up in aqueous NaHCO₃ (5 mL) washed with EtOAc (2×8 mL) and the combined organic layers washed with brine (10 mL), dried over MgSO₄, filtered and concentrated under reduced pressure. The solid was triturated from Et₂O and filtered to provide a pale yellow solid (10 mg, 17%). ¹H NMR (300 MHz, DMSO-d₆) δ 7.37-7.43 (m, 4H), 6.84 (d, J=14.24 Hz, 2H), 6.61 (d, J=8.14 Hz, 2H), 6.35 (t, J=6.10 Hz, 1H), 5.79 (s, 2H), 5.42 (s, 2H), 4.26 (d, J=6.10 Hz, 2H), 3.79 (q, J=6.89 Hz, 1 H), 3.54-3.61 (m, 1H), 1.96-2.01 (m, 2H), 1.66-1.84 (m, 2H). MS (ESI) positive ion 396 (M+H)⁺; negative ion 394 (M−H)⁻.

### EXAMPLE 41

### 6-[(2-butoxyethoxy)methyl]-5-{4-[(4-chlorobenzyl)amino]phenyl}pyrimidine-2,4-diamine

### EXAMPLE 41A

### 5-(4-Amino-phenyl)-6-(2-butoxy-ethoxymethyl)-pyrimidine-2,4-diamine

The titled compound was prepared according to the procedure described in Example 2, substituting tetrahydrofuran-2-carbonyl chloride for benzyloxyacetyl chloride used in Example 2.

### EXAMPLE 41B

### 6-[(2-Butoxyethoxy)methyl]-5-{4-[(4-chlorobenzyl)amino]phenyl}pyrimidine-2,4-diamine

To a stirred solution of 5-(4-amino-phenyl)-6-(2-butoxy-ethoxymethyl)-pyrimidine-2,4-diamine from Example 41A (140 mg, 0.536 mmol) in MeOH (5.3 mL) was added 4-chlorobenzaldehyde (75 mg, 0.536 mmol). After 30 minutes at room temperature, the reaction was cooled to 0° C. Glacial acetic acid (0.1 mL, 1.5 mmol) was added followed by NaCNBH₃ (37 mg, 0.588 mmol). The mixture was warmed to room temperature over 2 hours, the solvent was removed under reduced pressure and the residue taken up in saturated NaHCO₃ (10 mL). The solution was washed with EtOAc (2×10 mL) and the combined organic layers washed with brine (10 mL), dried over MgSO₄, filtered and concentrated under reduced pressure. Purification on reverse-phase HPLC (0-70% CH₃CN, aqueous NH₄OAc) provided a white powder (15 mg, 6%). ¹H NMR (300 MHz, DMSO-d₆) δ 7.36-7.43 (m, 4H), 6.89 (d, J=8.48 Hz, 2H), 6.60 (d, J=8.48 Hz, 2H), 6.40 (t, J=5.93 Hz, 1H), 6.10 (s, 2H), 5.78 (s, 2H), 4.27 (d, J=5.76 Hz, 2H), 3.93 (s, 2H), 3.35-3.39 (m, 4H), 3.29-3.31 (m, 2H), 1.37-1.46 (m, 2H), 1.20-1.32 (m, 2H), 0.845 (t, J=7.29 Hz, 3 H). MS (ESI) positive ion 456 (M+H)⁺; negative ion 454 (M−H)⁻.

### EXAMPLE 42

### 6-[(benzyloxy)methyl]-5-{4-[(1-ethylpropyl)amino]phenyl}pyrimidine-2,4-diamine

The titled compound was synthesized according to the procedure described in Example 2, substituting 3-pentanone for 4-chlorobenzaldehyde. ¹H NMR (300 MHz, DMSO-d₆) δ 0.90 (t, J=7.29 Hz, 6 H), 1.49 (m, 4 H), 3.17 (m, 1 H), 3.98 (s, 2 H), 4.35 (s, 2 H), 5.42 (d, J=8.14 Hz, 1 H), 5.50 (s, 2 H), 5.86 (s, 2 H), 6.58 (d, J=8.48 Hz, 2 H), 6.88 (d, J=8.48 Hz, 2 H), 7.20 (m, 2 H), and 7.27 (m, 3 H). MS (ESI) positive ion 392 (M+H)⁺; negative ion 390 (M−H)⁻.

### EXAMPLE 43

### 4-{[(4-{2,4-diamino-6-[(benzyloxy)methyl]pyrimidin-5-yl}phenyl)amino]methyl}benzonitrile

The titled compound was prepared according to the procedure described in Example 2, substituting 4-cyano-benzaldehyde for 4-chloro-benzaldehyde. ¹H NMR (300 MHz, DMSO-d₆) δ 7.80 (d, J=8.1 Hz, 2H), 7.59 (d, J=8.5 Hz, 2H), 7.28-7.15 (m, 5H), 6.90 (d, J=8.5 Hz, 2H), 6.59 (d, J=8.5 Hz, 2H), 6.48 (t, J=6.1 Hz, 1H), 5.87 (s, 2H), 5.47 (bs, 2H), 4.39 (d, J=6.1 Hz, 2H), 4.31 (s, 2H), 3.93 (s, 2H). MS (ESI) positive ion 437 (M+H)⁺; negative ion 435 (MH)⁻.

### EXAMPLE 44

### 4-{[(4-{2,4-diamino-6-[(benzyloxy)methyl}pyrimidin-5-yl}phenyl)(methyl)amino]methyl}benzonitrile

NaBH₃CN (5 mg, 0.08 mmol) was added to a mixture of 4{[(4-{2,4-diamino-6-[(benzyloxy)methyl]pyrimidin-5-yl}phenyl)amino]methyl}benzonitrile from Example 43 (22 mg, 0.05 mmol), 37% formaldehyde (5 μL, 0.06 mmol) and acetic acid (5 μL) in methanol (1 mL). The mixture was stirred at room temperature for 2 hours after which another portion of acetic acid (5 μL), formaldehyde (5 μL, 0.08 mmol), and NaBH₃CN (5 mg, 0.06 mmol) was added and stirred for another hour. The mixture was partitioned between ethyl acetate and aqueous NaHCO₃ (20 mL, 1:1). The organic phase was washed with brine, dried (MgSO₄), filtered and concentrated under reduced pressure. The residue was purified on silica gel with ethyl acetate/methanol (10/1) to provide the titled compound (15 mg). ¹H NMR (300 MHz, DMSO-d₆) δ 7.79 (d, J=8.5 Hz, 2H), 7.43 (d, J=8.1 Hz, 2H), 7.30-7.15 (m, 5H), 7.00 (d, J=8.8 Hz, 2H), 6.73 (d, J=8.8 Hz, 2H), 5.89 (s, 2H), 5.51 (bs, 2H), 4.68 (s, 2H), 4.32 (s, 2H), 3.95 (s, 2H), 3.06 (s, 3H). MS (ESI) positive ion 451 (M+H)+; negative ion 449 (M−H)⁻.

### EXAMPLE 45

### 5-{4-[(4-chlorobenzyl)amino]phenyl}-6-[(3-methylbutoxy)methyl]pyrimidine-2,4-diamine

### EXAMPLE 45A

### 3-(Methylbutoxy)acetic acid.

Allyl isoamyl glycolate (0.5 g, 2.68 mmol) was dissolved in MeOH (6 mL) and 2 M NaOH (6 mL) was added. After 1 hour, the mixture was concentrated under reduced pressure and the remainder acidified with 1 M HCl to pH 3, The solution was extracted with EtOAc (3×10 mL) and the combined organic layers washed with brine, dried over MgSO₄ filtered and concentrated to provide the titled compound as a clear oil (371 mg, 95%).

### EXAMPLE 45B

### 3-(Methylbutoxy)acetyl chloride.

3-(Methylbutoxy)acetic acid (1.6 g, 10.9 mmol) was dissolved in SOC1₂ (8 mL, 100 mmol) and heated to reflux for 3 hours. The mixture was cooled to room temperature and concentrated under reduced pressure. The resulting acid chloride was taken on to the next step without further purification.

### EXAMPLE 45C

### 4-(3-Methylbutoxy)-2-(4-nitrophenyl)-3-oxo-butvronitrile.

To a solution of 4-nitrophenylacetonitrile (500 mg, 3.0 mmol) in CH₂Cl₂ (5 mL) at 0° C. was added Et₃N (0.86 mL, 6.0 mmol) and DMAP (38 mg, 0.3 mmol). A solution of 3-(methylbutoxy)acetyl chloride (10 mmol) from Example 46B in CH₂Cl₂ (2 mL) was slowly added. The reaction was warmed to room temperature and stirred for 1 hour. The mixture was diluted with EtOAc (20 mL) and washed with 1 M HCl (10 mL), brine (10 mL), dried over MgSO₄, filtered and concentrated to provide the titled compound as a dark green solid (580 mg, 66%).

### EXAMPLE 45D

### 6-(3-Methylbutoxymethyl)-5-(4-nitrophenyl)-pyrimidine-2,4-diamine

To a solution of 4-(3-methylbutoxy)-2-(4-nitrophenyl)-3-oxo-butyronitrile (580 mg, 2.0 mmol) from Example 45C in CH₂Cl₂ (4.5 mL) and MeOH (0.5 mL) at 0° C. was added trimethylsilyl-diazomethane (2.0 M in Et₂O, 3 mL, 6.0 mmol). The reaction was stirred at room temperature for 1 hour. Glacial acetic acid (3 mL) was slowly added to quench excess TMS-diazomethane. The mixture was diluted with EtOAc (20 mL) and washed with aqueous NaHCO₃ solution (2×10 mL), brine (10 mL), dried over Mg SO₄, filtered and concentrated under reduced pressure. The residue was taken up in EtOH (10 mL) followed by the addition of guanidine HCl (190 mg, 2.0 mmol) in EtOH (2 mL) and KOEt (1.0 mL, 2.0 mmol). The mixture was heated to refluxed for 1 hour after which it was concentrated under reduced pressure, taken up in 2M NaOH (30 mL) and filtered. The resulting black solid was recrystallized from EtOH to provide the titled compound as a yellow solid (160 mg, 24%).

### EXAMPLE 45E

### 5-(4-Aminophenyl)-6-(3-methylbutoxymethyl)-pyrimidine-2,4-diamine

To a flask containing 6-(3-methylbutoxymethyl)-5-(4-nitrophenyl)-pyrimidine-2,4-diamine (150 mg, 0.453 mmol) from Example 45D was added 10% Pd/C (15 mg, 0.014 mmol) and glacial acetic acid (4.5 mL). The mixture was placed under an atmosphere of H₂ and stirred at room temperature for 4 hours. The mixture was filtered through Celite and concentrated under reduced pressure to provide the titled compound as a clear yellow oil (125 mg, 92%).

### EXAMPLE 45F

### 5-{4-[(4-Chlorobenzyl)amino]phenyl}-6-[(3-methylbutoxy)methyl]pyrimidine-2,4-diamine

To a solution of 5-(4-aminophenyl)-6-(3-methylbutoxymethyl)-pyrimidine-2,4-diamine from Example 45E (120 mg, 0.40 mmol) in MeOH (4 mL) was added 4-chlorobenzaldehyde (56 mg, 0.40 mmol). The mixture was stirred for 30 minutes at room temperature then cooled to 0° C. Glacial acetic acid (0.06 mL, 1.0 mmol) was added followed by NaCNBH₃ (28 mg, 0.44 mmol). The mixture was warmed to room temperature over 1 hour after which aqueous NaHCO₃ (8 mL) was added to the reaction. The mixture was extracted with EtOAc (2×15 mL) and the combined organic layers washed with brine (10 mL), dried over MgSO₄, filtered and concentrated. Purification on reverse-phase HPLC (0-70% CH₃CN, aqueous NH₄OAc) provided the titled compound as an off white powder (20 mg, 12%). ¹H NMR (300 MHz, DMSO-d₆) δ 7.35-7.41 (m, 4H), 6.88 (d, J=8.48 Hz, 2H), 6.60 (d, J=8.48 Hz, 2H), 6.35 (t, J=5.98 Hz, 1H), 5.95 (s, 2H), 5.62 (s, 2H), 4.26 (d, J=5.83 Hz, 2H), 3.83 (s, 2H), 3.21 (t, J=6.75 Hz, 2H), 1.50 1.57 (m, 1H), 1.24 (q, J=6.44 Hz, 2H), 0.774 (d, J=6.0 Hz, 6H). MS(ESI) positive ion 426 (M+H)⁺; negative ion 424 (M−H)⁻.

### EXAMPLE 46

### 5-(4-{[2-(benzyloxy)ethyl]amino}phenyl)-6-ethylpyrimidine-2,4-diamine

The titled compound was prepared according to the procedure described in Example 6, substituting 2-benzyloxyethanol for 4-cyanobenzyl alcohol used in Example 6. ¹H NMR (500 MHz, DMSO-d₆) δ ppm 0.96 (t, J=7.49 Hz, 3 H), 2.14 (q, J=7.59 Hz, 2 H), 3.26 (dd, J=11.54, 5.61 Hz, 2 H), 3.61 (t, J=5.77 Hz, 2 H), 4.53 (s, 2 H), 5.36 (s, 2 H), 5.67 (t, J=5.61 Hz, 1 H), 5.78 (s, 2 H), 6.66 (d, J=8.73 Hz, 2 H), 6.86 (d, J=8.42 Hz, 2 H), 7.34 (m, 5 H);MS (ESI positive ion 364(M+H)⁺.

### EXAMPLE 47

### 6-[(benzyloxy)methyl]-5-(4-{[(6-chloropyridin-3-yl)methyl]amino}phenyl)pyrimidine-2,4-diamine

The titled compound was prepared according to the procedure described in Example 2, substituting 6-chloro-pyridine-3-carbaldehyde (Oida, Sadao et al. Chem. Pharm. Bull.; EN; 48; 5; 2000; 694-707) for 4-chloro-benzaldehyde. ¹H NMR (300 MHz, DMSO-d₆) δ 8.45 (d, J=2.4 Hz, 1H), 7.85 (dd, J=8.5 Hz, J=2.4 Hz, 1H), 7.49 (d, J=8.5 Hz, 1H), 7.287.15 (m, 5H), 6.92 (d, J=8.8 Hz, 2H), 6.63 (d, J=8.8 Hz, 2H), 6.39 (t, J=6.1 Hz, 1H), 5.87 (s, 2H), 5.50 (bs, 2H), 4.32, 4.33 (s, s, 4H), 3.93 (s, 2H). MS (ESI) positive ion 447 (M+H)⁺; negative ion 445 (M−H)⁻.

### EXAMPLE 48

### N-benzyl-3-(2,6-diamino-5-{4-[(4-chlorobenzyl)amino]phenyl}pyrimidin-4-yl)propanamide

### EXAMPLE 48A

### N-Benzyl-3-[2,6-diamino-5-(4-nitrophenyl)-pyrimid-4-yl]-propionamide

To a solution of 3-[2,6-diamino-5-(4-nitrophenyl)pyrimidine-4-yl]-propionic acid hydrochloride from Example 57 B(50 mg, 0.147 mmol) in DMF (1.5 mL) was added benzylamine (0.032 mL, 0.29 mmol) and TBTU (50 mg, 0.155 mmol ). The mixture was stirred at room temperature for 16 hours, diluted with water and the resulting solid was filtered and rinsed with diethyl ether. The titled compound was collected as a bright yellow solid (47 mg, 82%).

### EXAMPLE 48B

### N-Benzyl-3-[2,6-diamino-5-(4-nitrophenyl)-pyrimid-4-yl]-propionamide

A mixture of N-Benzyl-3-[2,6-diamino-5-(4-nitrophenyl)-pyrimid-4-yl]-propionamide from Example 48A (45 mg, 0.115 mmol) and 10% Pd/C (5 mg) in jacial acetic acid (1 mL) was stirred under an atmosphere of H₂ for 3 hours at room temperature. The mixture was filtered through Celite, rinsed with MeOH and concentrated under reduced pressure. The titled compound was recovered as a white solid (40 mg, 96%).

### EXAMPLE 48C

### N-Benzyl-3-(2,6-diamino-5-{4-[(4-chlorobenzyl)amino]phenyl}pyrimidin-4-yl)propanamide

To a stirred solution of N-benzyl-3-[2,6-diamino-5-(4-nitrophenyl)-pyrimid-4-yl]-propionamide from Example 48B (40 mg, 0.11 mmol) in MeOH (1.1 mL) was added 4-chlorobenzaldehyde (15 mg, 0.11 mmol). After 30 minutes at room temperature, the reaction was cooled to 0° C., glacial acetic acid (0.03 mL, 0.5 mmol) was added followed by NaCNBH₃ (8 mg, 0..12 mmol). The mixture warmed to room temperature over 1 hour, diluted with saturated NaHCO₃ (5 mL) and extracted with EtOAc (2×5 mL). The combined organic layers were washed with brine (5 mL), dried over MgSO₄, filtered and concentrated under reduced pressure. Purification by reverse-phase HPLC (0-70% CH₃CN, aqueous NH₄OAc) provided an off white powder (10 mg, 18%). ¹H NMR (300 MHz, DMSO-d₆) δ 8.24 (t, J=5.93 Hz, 1H), 7.39-7.44 (m, 4H), 7.16-7.31 (m, 5H), 6.86 (d, J=8.48 Hz, 2H), 6.61 (d, J=8.48 Hz, 2H), 6.34 (t, 6.10,1H), 5.70 (s, 2H), 5.30 (s, 2H), 4.26 (d, J=6.10 Hz, 2H), 4.19 (d, J=5.76 Hz, 2H), 3.21-3.42 (m, 4H). MS (ESI) positive ion 487 (M+H)⁺; negative ion 485 (M−H)⁻.

### EXAMPLE 49

### 3-(2,6-diamino-5-{4-[(4-chlorobenzyl)amino]phenyl}pyrimidin-4-yl)-N-phenylpropanamide

### EXAMPLE 49A

### 3-[2,6-Diamino-5-(4-nitro-phenyl)-pyrimidin-4-yl]-N-phenyl-propionamide

The titled compound was prepared according to the procedure described in Example 57A-C, substituting aniline for n-butylamine used in Example 57C.

### EXAMPLE 49B

### 3-(2,6-Diamino-5-{4-[(4-chlorobenzyl)amino]phenyl}pyrimidin-4-yl)-N-phenylpropanamide

3-(2,6-Diamino-5-({4-nitro-phenyl}pyrimidin-4-yl)-N-phenylpropanamide from Example 49A (55 mg, 0.145 mmol) was combined with 10% Pd/C (6 mg,) in glacial acetic acid (1.4 mL) and placed under an atmosphere of H₂. The mixture was stirred for 3.5 hours at room temperature, filtered through Celite, rinsed with MeOH and concentrated under reduced pressure. The residue was dissolved in MeOH (1.4 mL) and 4-chlorobenzaldehyde (20 mg, 0.145 mmol) was added. The mixture was cooled to 0° C., glacial acetic acid (0.03 mL) and NaCNBH₃ were added. The mixture was warmed to room temperature, stirred for 1.5 hour, diluted with aqueous NaHCO₃ (5 mL) and extracted with EtOAc (2×10 -nL). The combined organic layers were washed with brine, dried over MgSO₄, filtered and concentrated under reduced pressure. The residue was purified by reverse-phase HPLC (0-70% CH₃CN, aqueous NH₄OAc) to provide a white solid (10 mg, 15%). ¹H NMR (300 MHz, DMSO-d₆) δ 9.86 (s, 1H), 7.53 (d, J=7.80 Hz, 2H), 7.38-7.43 (m, 4H), 7.25 (t, J=7.97 Hz, 2H), 6.99 (t, J=7.29 Hz, 1H), 6.88 (d, J=8.48 Hz, 2H), 6.61 (d, J=8.48 Hz, 2H), 6.35 (t, J=5.76, 1H), 5.82 (s, 2H), 5.43 (s, 2H), 4.25 (d, J=5.76 Hz, 2H), 2.53-2.57 (m, 2H), 2.41-2.46 (m, 2H). MS (ESI) positive ion 473 (M+H)⁺; negative ion 471 (M−H)⁻.

### EXAMPLE 50

### 6-[(benzyloxy)methyl]-5-{4-[(1-pyridin-4-ylethyl)amino]phenyl}pyrimidine-2,4-diamine

NaBH₃CN (10 mg, 0.15 mmol) was added to a mixture of 5-(4-amino-phenyl)-6-benzyloxymethyl-pyrimidine-2,4-diamine from Example 2 (32 mg, 0.1 mmol), 1-pyridin-4-yl-ethanone (12 μL, 0.11 mmol) and acetic acid (10 μL) in methanol (2 mL). The mixture was heated to 50° C. for 2 hours afterwhich three more portions of reagents (acetic acid (10 μL×3), 1-pyridin-4-yl-ethanone (12 mL×3) and NaBH₃CN (10 mg×3)) were added with an interval of 1 hour. The mixture was then cooled to room temperature, partitioned between ethyl acetate and aqueous NaHCO₃ (30 mL, 1:1). The separated organic phase was washed with brine, dried (MgSO₄), filtered and concentrated under reduced pressure. The residue was purified on silica gel with ethyl acetate/methanol (10/1) to provide the titled compound (10 mg). ¹H NMR (300 MHz, DMSO-d₆) δ 8.49 (d, J=6.1 Hz, 2H), 7.42 (d, J=6.1 Hz, 2H), 7.30-7.12 (m, 5H), 6.85 (d, J=8.5 Hz, 2H), 6.53 (d, J=8.5 Hz, 2H), 6.35 (d, J=6.8 Hz, 1H), 5.87 (s, 2H), 5.45 (bs, 2H), 4.54 4.45 (m, 1H), 4.29 (s, 2H), 3.90 (s, 2H), 1.44 (d, J=7.1 Hz, 2H). MS (ESI) positive ion 427 (M+H)⁺; negative ion 425 (M−H)⁻.

### EXAMPLE 51

### 4-}1-[(4-{2,4-diamino-6-[(benzyloxy)methyl]pyrimidin-5-yl]phenyl)amino]ethyl}benzonitrile

The titled compound was synthesized according to the procedure described in Example 50, substituting 4-cyanoacetophenone for 4-acetylpyridine. ¹H NMR (300 MHz, DMS-d₆) δ 1.43 (d, J=6.78 Hz, 3H), 3.90 (s, 2 H), 4.28 (s, 2 H), 4.58 (t, J=6.60 Hz, 1 H), 5.45 (s, 2 H), 5.87 (s, 2 H), 6.39 (d, J=6.44 Hz, 1 H), 6.51 (d, J=8.82 Hz, 2 H), 6.84 (d, J=8.48 Hz, 2 H), 7.13 (dd, J=6.95, 2.88 Hz, 2 H), 7.25 (m, 3 H), 7.62 (d, J=8.48 Hz, 2 H), and 7.78 (d, J=8.14 Hz, 2 H). MS (ESI) positive ion 451 (M+H)⁺; 449 (M−H)⁻.

### EXAMPLE 52

### 6-[(benzyloxy)methyl]-5-(4-{[1-(4-chlorophenyl)ethyl]amino}phenyl)pyrimidine-2,4-diamine

The titled compound was synthesized according to the procedure described in Example 50, substituting 4-chloroacetophenone for 4-acetylpyridine. ¹H NMR (500 MHz, DMSO-d₆) δ 1.41 (d, J=6.71 Hz, 3H), 3.90 (s, 2H), 4.29 (d, J=2.44 Hz, 2H), 4.48 (pentet, J=6.60 Hz, 1 H), 5.46 (s, 2H), 5.88 (s, 2H), 6.30 (d, J=6.71 Hz, 1H), 6.52 (d, J=8.54 Hz, 2 H), 6.84 (d, J=8.54 Hz, 2 H), 7.14 (dd, J=7.17, 2.29 Hz, 2 H), 7.24 (m, 3 H), 7.36 (m, 2 H), and 7.44 (m, 2 H). MS (ESI) positive ion 460 (M+H)⁺; negative ion 458 (M−H)⁻.

### EXAMPLE 53

### 6-[(benzyloxy)methyl}-5-{4-[(4-methoxybenzyl)amino]phenyl}pyrimidine-2,4-diamine

The titled compound was prepared according to the procedure described in Example 2, substituting 4-methoxy-benzaldehyde for 4-chloro-benzaldehyde. ¹H NMR (300 MHz, DMSO-d₆) δ 7.32 (d, J=8.5 Hz, 2H), 7.28-7.15 (m, 5H), 6.90 (d, J=8.5 Hz, 2H), 6.89 (d, J=8.5 Hz, 2H), 6.62 (d, J=8.5 Hz, 2H), 6.22 (t, J=5.8 Hz, 1H), 5.87 (s, 2H), 5.47 (bs, 2H), 4.33 (s, 2H), 4.19 (d, J=5.8 Hz, 2H), 3.95 (s, 2H), 3.72 (s, 3H). MS (ESI) positive ion 442 (M+H)⁺; negative ion 440 (M−H)⁻.

### EXAMPLE 54

### 6-[(benzyloxy)methyl]-5-{4-[(cyclohexylmethyl)amino]phenyl}pyrimidine-2,4-diamine

The titled compound was prepared according to the procedure described in Example 2, substituting cyclohexanecarbaldehyde for 4-chloro-benzaldehyde. ¹H NMR (300 MHz, DMSO-d₆) δ 7.32-7.18 (m, 5H), 6.90 (d, J=8.5 Hz, 2H), 6.59 (d, J=8.8 Hz, 2H), 5.86 (s, 2H), 5.70 (t, J=5.8 Hz, 1H), 5.49 (bs, 2H), 4.35 (s, 2H), 3.97 (s, 2H), 2.86 (t, J=5.8 Hz, 2H), 1.880.90(m, 11H). MS (ESI) positive ion 418 (M+H)⁺; negative ion 416 (M−H)⁻.

### EXAMPLE 55

### 3-(2,6-diamino-5-{4-[(4-chlorobenzyl)amino]phenyl}pyrimidin-4-yl)-N-(3-methylphenyl)propanamide

The titled was prepared according to the same procedure described for Example 57, substituting 16 mg (0.15 mmol) of m-toluidine for n-butylamine used in Example 57C. The yield was 10 mg (9%) of the TFA salt as a foam. ¹H NMR (300 MHz, d₆-DMSO) mixture of rotamers δ 11.87 (s, 1H), 9.86 (s, 1H), 8.10 (s, 1H), 7.35 (m, 9.5H), 7.29 (t, 1H, J=7.6 Hz), 6.90 (m, 3.5H), 6.64 (m, 4H), 4.26 (m, 2H), 2.50 (m, 4H), 2.26 (s, 3H), 2.26 (s, minor, 3H); MS (ESI) m/z 485 [M−H]⁺.

### EXAMPLE 56

### 6-[(benzyloxy)methyl]-5-{4-[(4-chlorobenzyl)oxy]phenyl}pyrimidine-2,4-diamine

### EXAMPLE 56A

### 4-(2,4-Diamino-6-benzyloxymethyl-pyrimidin-5-yl)-phenol

To 654 mg (2.03 mmol) of 5-(4-amino-phenyl)-6-benzyloxymethyl-pyrimidine-2,4-diamine from Example 2 was added 7 mL of 1M H₂SO₄. The solution was stirred at ambient temperature until all of the starting aniline had dissolved, then it was cooled with an ice bath. To the cold suspension was added a solution of 168 mg (2.43 mmol) of sodium nitrite dissolved in a minimum amount of water, and the reaction was stirred for 10 minutes at 0° C., warmed to ambient temperature over 10 minutes, then heated to reflux for 40 minutes. The reaction was cooled, treated with 10 mL of ethyl acetate and 15 mL of saturated NaHCO₃. A gummy precipitate formed which could be dissolved in a small amount of methanol, then partitioned between the aqueous and organic layers to speed dissolution. The aqueous layer was extracted with additional ethyl acetate (2×10 mL), then back extracted with brine (1×10 mL), dried over MgSO₄, filtered, and concentrated to a foam. The residue was taken up in methanol and reconcentrated to provide the titled phenol (600 mg, 92%) as a yellow foam.

### EXAMPLE 56B

### 6-Benzyloxymethyl-5-[4-(4-chloro-benzyloxy)-phenyl]-pyrimidine-2,4-diamine

To a solution of 48 mg (0.15 mmol) of 4-(2,4-diamino-6-benzyloxymethyl-pyrimidin-5-yl)-phenol from Example 56A in 0.5 mL of ethanol was added 0.15 mmol of potassium ethoxide in 60 μL of ethanol. The solution was stirred for 2 minutes, then 31 mg (0.15 mmol) of 4-chlorobenzyl bromide was added. The reaction was stirred for 4.5 hour then 1 mL of water was added, and a yellow precipitate formed. The precipitate was collected, washed with water, then with diethyl ether, and dried on the filter to provide 44 mg (67%) of a pale yellow solid. Similar products prepared from other halides could be purified by recrystallization from i-PrOH/H₂O or ethanol/H₂O. Alternatively, the products could be purified by reverse phase HPLC, eluting with a 5 to 100% CH₃CN in 0.1% aq. TFA gradient to give the final compounds as its TFA salt. ¹H NMR (300 MHz, d₆-DMSO) δ 7.49 (m, 4H), 7.28 (m, 3H), 7.17 (m, 4H), 7.03 (d, 2H, J=8.8 Hz), 5.95 (s, 2H), 5.58 (s, 2H), 5.12 (s, 2H), 4.32 (s, 2H), 3.94 (s, 2H); MS (ESI) m/z 447 [M+H]⁺.

### EXAMPLE 57

### N-butyl-3-(2,6-diamino-5-{4-[(4-chlorobenzyl)amino]phenyl}pyrimidin-4-yl)propanamide

### EXAMPLE 57A

### 3-[2,6-Diamino-5-(4-nitro-phenyl)-pyrimidin-4-yl]-propionic acid methyl ester

To an ice-cooled solution of 3.24 g (20.0 mmol) of 4-nitrophenylacetonitrile and 130 mg (1.06 mmol) of 4-N,N-dimethylaminopyridine in 40 mL of CH₂Cl₂ was added 8.4 mL (60 mmol) of triethylamine, followed by 4.0 mL (32 mmol) of methyl 4-chloro-4-oxobutyrate dropwise over 1 minute. The mixture was stirred at 0° C. for 1 hour, and concentrated under reduced pressure. The residue was taken up in 80 mL of 0.5M HCl, and extracted with ethyl acetate (3×40 mL). The combined organic layers were back extracted with brine (1×40 ML), dried over MgSO₄, and filtered. The solution was cooled with an ice bath, 25 mL of methanol was added, followed by 25 mL of 2M trimethylsilyldiazomethane in diethyl ether. The solvents were removed under reduced pressure and the oily residue triturated with methanol to give a granular solid afferwhich the solvent was removed under reduced pressure. The solid was dissolved in 40 mL of tetrahydrofuran, afterwhich a premixed solution of 1.91 g (20 mmol) of guanidine hydrochloride and 20 mL of sodium methoxide in 25 mL of methanol, containing some solid KCl was added. The mixture was heated to reflux for 15 minutes, cooled and concentrated under reduced pressure. The residue was taken up in 50 mL of water, and filtered. The precipitate was washed with 10 mL of water, and then with 25 mL of methanol. The crude product was recrystallized from 40 mL of methanol to provide the titled compound (700 mg, 11%) as a yellow powder.

### EXAMPLE 57B

### 3-[2,6-Diamino-5-(4-nitro-phenyl)-pyrimidin-4-yl]-propionic acid

To 694 mg (2.19 mmol) of 3-[2,6-diamino-5-(4-nitro-phenyl)-pyrimidin-4-yl]-propionic acid methyl ester from Example 57A was added 25 mL of 1M HCl. The suspension was heated to 90° C. for 1 hour during which time the starting ester dissolved. The mixture was concentrated under reduced pressure to provide the titled compound (762 mg, 100%) of the product as a light brown solid containing a small amount of water.

### EXAMPLE 57C

### N-Butyl-3-{2,6-diamino-5-[4-(4-chloro-benzylamino)-phenyl]-pyrimidin-4-yl}-propionamide

To 37 mg (0.50 mmol) of n-butylamine was added a solution of 50 mg (0.15 mmol) of 3-[2,6-diamino-5-(4-nitro-phenyl)-pyrimidin-4-yl]-propionic acid and 50 mg (0.16 mmol) of O-benzotriazol-1-yl-N,N,N′,N′-tetramethyluronium tetrafluoroborate (TBTU) in 1 mL of DMF. The mixture was shaken at ambient temperature for 18 hours, diluted with 5 mL of water and 1 mL of saturated NaHCO₃. The precipitated amide was filtered, washed with water, dried on the filter. To the crude amide was added 5 mg of 10% Pd-C and 1 mL of acetic acid. The mixture was stirred under 1 atmosphere of H₂ for 4 hours, then filtered. The acetic acid was removed under reduced pressure. To the residue was added 11 mg of 4-chlorobenzaldehyde (0.079 mmol), 0.5 mL of methanol, and 0.5 mL of acetic acid. The solution was stirred for 10 minutes afterwhich 20 mg (0.32 mmol) of sodium cyanoborohydride was added. The mixture was stirred for 30 minutes at ambient temperature, then the mixture was concentrated under reduced pressure. The residue was taken up in aqueous NaHCO₃ (3 mL) and extracted with ethyl acetate (2×1 mL). The combined ethyl acetate layers were back extracted with brine (1×1 mL), dried over MgSO₄, filtered, and concentrated under reduced pressure to provide the crude benzylamine. The product was purified by reverse phase HPLC, eluting with a 5 to 100 CH₃CN/aq. 0.1% trifluoroacetic acid gradient to provide 15 mg (15%) of Nbutyl-3-{2,6-diamino-5-[4-(4-chloro-benzylamino)-phenyl]-pyrimidin-4-yl}-propionamide TFA salt as a foam. ¹H NMR (300 MHz, d₆-DMSO) δ 11.94 (s, 1H), 8.07 (s, 1H), 7.87 (t, 1H, J=5.4Hz), 7.39 (m, 4H), 6.91 (d, 2H, J=8.8 Hz), 6.65 (d, 2H, J=8.8 Hz), 6.61 (s, 2H), 4.28 (s, 2H), 3.00 (m, 2H), 2.44 (t, 2H, J=7.1 Hz), 2.27 (t, 2H, J=7.0 Hz), 1.33 (m, 2H), 1.22 (m, 2H), 0.85 (t, 3H, J=7.3 Hz); MS (ESI) m/z 453 [M+H]⁺.

### EXAMPLE 58

### 6-[(benzyloxy)methyl]-5-(4-{[(4-chlorobenzyl)amino]methyl}phenyl)pyrimidine-2,4-diamine

### EXAMPLE 58A

### 4-(2,4-Diamino-6-benzyloxymethyl-pyrimidin-5-yl)-benzonitrile

The titled compound was prepared according to the procedure described in Example 2, substituting 4-cyanophenylacetonitrile for 4-nitrophenylacetonitrile used in Example 2A. ¹HNMR (DMSO-d₆, 300 MHz), δ 7.81 (d, J=8.5 Hz, 2H), 7.40 (d, J=8.5 Hz, 2H), 7.32-7.23 (m, 3H), 7.15-7.09 (m, 2H), 6.11 (s, 2H), 5.85 (s, 2H), 4.30 (s, 2H), 3.95 (s, 2H); MS (ESI) m/e 332 (M+H)⁺.

### EXAMPLE 58B

### 5-(4-Aminomethyl-phenyl)-6-benzyloxymethyl-pyrimidine-2,4-diamine

To a stirred suspension of phenylcyanide (600 mg, 1.8 mmol) from Example 58A in 1.0 N of NH₃/MeOH was added Raney Ni (75 mg, prewashed with MeOH and THF). The reaction flask was capped with a hydrogen balloon and hydrogenated at 60° C. for 4 hours. Thealmost clear solution was cooled to ambient temperature, filtered through celite, concentrated under reduced pressure to provide the titled compound as beige solid (450 mg, 74% yield). ¹HNMR (DMSO-d₆, 300 MHz), δ 7.36 (d, J=8.1 Hz, 2H), 7.34-7.12 (m, 5H), 7.15 (d, J=8.1 Hz, 2H), 5.97 (s, 2H), 5.55 (s, 2H), 4.44 (br m, 2H), 4.33 (s, 2H), 3.96 (s, 2H); MS (ESI) m/e 336(M+H)⁺.

### EXAMPLE 58C

### 6-[(Benzyloxy)methyl]-5-(4-{[(4-chlorobenzyl)amino]methyl}phenyl)pyrimidine-2,4-diamine

The titled compound was prepared according to the procedure described in Example 2D, substituting benzylamine from Example 58B for the aniline used in Example 2D. ¹HNMR (DMSO-d₆, 300 MHz), δ 7.45-7.12 (m, 13H), 5.97 (s, 2H), 5.57 (s, 2H), 5.24 (brm, 1H), 4.48 (s, 2H), 4.32 (s, 2H), 3.96 (s, 2H), 3.70 (s, 2H); MS(ESI) m/e 460, 462 (M+H)⁺.

### EXAMPLE 59

### 5-[4-(benzylamino)phenyl]-6-[(benzyloxy)methyl]pyrimidine-2,4-diamine

The title compound was synthesized according to the procedure described in Example 2, substituting benzaldehyde for 4-chlorobenzaldehyde. ¹H NMR (400 MHz, DMSO-d₆) δ 3.95 (s, 2 H), 4.28 (d, J=5.83 Hz, 2 H), 4.33 (s, 2 H), 5.48 (s, 2 H), 5.85 (s, 2 H), 6.29 (t, J=5.89 Hz, 1 H), 6.63 (d, J=8.59 Hz, 2 H), 6.89 (d, J=8.59 Hz, 2 H), 7.18 (dd, J=7.83, 1.69 Hz, 2 H), 7.26 (m, 4 H), 7.34 (m, 2 H), and 7.40 (m, 2 H). MS (ESI) positive ion 412 (M+H)⁺; negative ion 410 (M−H)⁻.

### EXAMPLE 60

### 6-[(benzyloxy)methyl]-5-(4-{[(4-nitrophenyl)amino]methyl}phenyl)pyrimidine-2,4-diamine

A mixture of benzylamine (30 mg, 0.089 mmol) from Example 58B, diisopropylethylamine in excess (150 μL), 1-fluoro-4-nitrobenzene (19 μL, 0.18 mmol) in 1.0 mL of NMP was heated at 200° C. for 20 minutes in a Personal Chemistry Optimizer MicroWave reactor. Solvent was removed on a Savant Speed Vac, and the crude residue was purified on a preparative TLC to provide the titled compound as a light yellow solid (10 mg, 24% yield). ¹HNMR (DMSO-d₆, 300MHz), δ 7.98 (d, J=9.5 Hz, 2H), 7.86 (t, J=6.1 Hz, 1H), 7.37 (d, J=8.1 Hz, 2H), 7.30-7.09 (m, 9H), 6.7 (d, J=9.5 Hz, 2H), 6.01 (s, 2H), 5.58 (s, 2H), 4.46 (d, J=6.1 Hz, 2H), 4.29 (s, 2H), 3.94 (s, 2H); MS (ESI) m/e 460, 462 (M+H)⁺.

### EXAMPLE 61

### 4-{[(4-{2,4-diamino-6-[(cyclobutylmethoxy)methyl]pyrimidin-5-yl}phenyl)amino]methyl}benzonitrile

### EXAMPLE 61A

### Cyclobutylmethoxyacetic acid

To NaH (1.72 g of 60%) in THF (10 ml) was added a solution of cyclobutanemethanol (3.0 g, 35 mmol) in THF (10 ml) at −15° C. The mixture was stirred at 25° C. for 1 hour, concentrated under reduced pressure afterwhich sodium chloroacetate (5.2 g, 45 mmol) in DMSO (100 ml) was added. The mixture was stirred at room temperature for 20 hours, then diluted with 300 ml water and extracted with hexane (100 ml×2). The aqueous phase was acidified with 2N HCl to pH 2, and then extracted with ethyl acetate (100 ml×2). The combined ethyl acetate layers were washed twice with H₂O (100 ml) and dried over MgSO₄, filtered and concentrated under reduced pressure to provide the titled cyclobutylmethoxyacetic acid as a pale yellow oil (4.2 g, 83%), which was used in the next step without purification.

### EXAMPLE 61B

### 1-Cyano-1-(4-nitropheny)-3-cyclobutylmethoxyacetone

To the cyclobutylmethoxyacetic acid from Example 61A (1.44 g, 10 mmol) in CH₂Cl₂ (20 ml) was added slowly oxalylchloride (2.54 g, 20 mmol) and DMF (0.1 ml) at 0° C. The mixture was stirred at 0° C. for 0.5 hour then at room temperature for Ihour before being concentrated under reduced pressure. The residue in fresh CH₂Cl₂ (10 ml) was added to a solution of nitrophenyl acetonitrile (1.62, 10 mmol) in CH₂Cl₂ with Et₃N (1.68 ml, 12 mmol) and DMAP (162 mg). The mixture was stirred overnight, then H₂O (30 ml) and HCl (2N, 1 ml) were added. The organic layer was washed with brine (2×10 ml), dried over MgSO4, filtered, concentrated under reduced pressures and purification by crystallization using ethyl acetate and hexane to provided the titled 1-cyano-1-(4-nitropheny)-3-cyclobutylmethoxy acetone (1.87 g, 65%). ¹H NMR (300 MHz, DMSO-D₆) δ 8.23 (d, J=9.0 Hz, 2H), 7.96 (d, J=9.0 Hz, 2H), 4.39 (s, 2H), 3.48 (d, J=9.0 Hz, 2H), 2.57 (m, 1H), 2.51 (s, 1H), 2.04-1.71 (m, 6H). MS (ESI) positive ion 287 (M+H)⁺.

### EXAMPLE 61C

### 2,4-Diamino-6-[(cyclobutylmethoxy)methyl]-5-(4-nitrophenyl)pyrimidine

To 1-cyano-1-(4-nitropheny)-3-cyclobutylmethoxyacetone from Example 61B (450 mg, 1.56 mmol) was added CH₂N₂ (3 mmol in 10 ml ether) at 0° C. The mixture was stirred for 10 minutes then the solvent removed under reduced pressure to provide a vinyl ether compound (470 mg, 99%). ¹H NMR (300 MHz, DMSO-D₆) δ 8.26 (d, J=9.0 Hz, 2H), 7.89 (d, J=9.0 Hz, 2H), 4.63 (s, 2H), 4.08 (s, 3H), 3.54 (d, J=9.0 Hz, 1H), 2.59 (m, 1H), 2.041.71 (m, 6H). MS (ESI) positive ion 301 (M+H)⁺′ negative ion 299 (M−H⁻. To the residue (453 mg, 1.5 mmol) in ethanol (20 mL) was added guanidine carbonate (270 mg, 1.5 mmol). The mixture was refluxed for 2 hours, then cooled to room temperature and filtered. The solid was washed with ethyl acetate (10 ml×3) and dried to provide the title compound as pale yellow crystals (385 mg, 78%).¹H NMR (300 MHz, DMSO-D₆) δ 8.24(d, J=9.0 Hz, 2H), 7.51 (d, J=9.0 Hz, 2H), 6.14 (s, 2H), 3.91 (s, 2H), 3.17 (d, J=9.0 Hz, 1H), 2.31 (m, 1H), 1.90-1.49 (m, 6H). MS (ESI) positive ion 330 (M+H)⁺, negative ion 328 (M−H)⁻.

### EXAMPLE 61D

### 2,4-Diamino-6-[(cyclobutylmethoxy)methyl]-5-(4-aminophenyl)pyrimidine

To 2,4-diamino-6-[(cyclobutylmethoxy)methyl]-5-(4-nitrophenyl)pyrimidine from Example 61C (380 mg, 1.15 mmol) in 10 ml methanol was added Pd/C (122 mg, 10%). The mixture was stirred under 1 atmosphere of hydrogen at room temperature for 2hours, then filtered and concentrated under reduced pressure to provide the titled compound (330 mg, 96%). ¹H NMR (300 MHz, DMSO-d₆) δ 6.85 (d, J=9.0 Hz, 2H), 6.60 (d, J=9.0 Hz, 2H), 5.85 (s, 2H), 5.20 (s, 2H), 5.11 (s, 2H), 3.85 (s, 2H), 3.21 (d, J=9.0 Hz, 1H), 2.39 (m, 1H), 1.95-1.59 (m, 6H). MS (ESI) positive ion 300 (M+H)⁺, negative ion 298(M−H)⁻.

### EXAMPLE 61E

### 4-{[(4-{2,4-Diamino-6-[(cyclobutylmethoxy)methyl]pyrimidin-5-yl}phenyl)amino]methyl}benzonitrile

To 2,4-diamino-6-[(cyclobutylmethoxy)methyl]-5-(4-aminophenyl)pyrimidine from Example 61D (30 mg, 0.1 mmol) in methanol (2 mL) and a buffer solution of acetic acid and sodium acetate (1 mL, pH 4-5) was added 4-cyanobenzaldehyde (14.5 mg, 0.11 mmol) followed by NaBH₃CN (76 mg, 0.12 mmol). The mixture was stirred at room temperature for 2 hours afterwhich the solvents were removed under reduced pressure. The residue was purified by column chromatography to provide the titled compound (23 mg, 55%). ¹H NMR (300 MHz, DMSO-d₆) δ 7.80 (d, J=9.0 Hz, 2H), 7.58 (d, J=9.0 Hz, 2H), 6.90 (d, J=9.0 Hz, 2H), 6.59 (d, J=9.0 Hz, 2H), 6.49 (t, J=3.0 Hz,1H), 5.98 (s, 2H), 5.62(s, 2H), 4.40 (d, J=6.0 Hz, 2H), 3.84 (s, 2H), 3.18 (d, J=6.0 Hz, 2H), 2.35 (m, 1H), 1.91-1.54 (m, 6H). MS (ESI) positive ion 415 (M+H)⁺; negative ion 413 (M−H)⁻.

### EXAMPLE 62

### tert-butyl 2-[(4-{2,4-Diamino-6-[(benzyloxy)methyl]pyrimidin-5-yl]phenyl)amino]ethylcarbamate

The title compound was synthesized according to the procedure described in Example 2, substituting tert-butyl N-(2-oxoethyl)carbamate for 4-chlorobenzaldehyde. ¹H NMR (300 MHz, DMSO-d₆) δ 1.39 (s, 9 H), 3.09 (m, 4 H), 4.03 (s, 2 H), 4.38 (s, 2H), 5.76 (s, 2 H), 6.24 (s, 2 H), 6.61 (d, J=8.48 Hz, 2 H), 6.90 (m, 1 H), 6.92 (d, J=8.48 Hz, 2 H), 7.23 (m, 2 H), and 7.30 (m, 3 H). MS (ESI) positive ion 465 (M+H)⁺; negative ion 463 (M−H)⁻ and 389 (M-75)⁻.

### EXAMPLE 63

### 4-[(4-{2,4-diamino-6-[(benzyloxy)methyl]pyrimidin-5-yl}phenoxy)methyl]benzonitrile

The titled compound was prepared by the same procedure described in Example 63, substituting 4-cyanobenzyl bromide for 4-chlorobenzyl bromide used in Example 63B. The product was purified by recrystallization from i-PrOH/H₂O or ethanol/H20 to give 40 mg (58%) of a yellow solid. ¹H NMR (300 MHz, d₆-DMSO) δ 7.92 (d, 2H, J=8.1 Hz), 7.65 (d, 2H, J=8.1 Hz), 7.26 (m, 3H), 7.15 (m, 4H), 7.05 (m, 2H), 5.94 (s, 2H), 5.56 (s, 2H), 5.25 (s, 2H), 4.28 (s, 2H), 3.92 (s, 2H); MS (ESI) m/z 438 [M+H]⁺.

### EXAMPLE 64

### 5-{4-[(4-chlorobenzyl)amino]phenyl}-6-[(tetrahydro-2H-pyran-2-ylmethoxy)methyl]pyrimidine-2,4-diamine

### EXAMPLE 64A

### 5-(4-Amino-phenyl)-6-(tetrahydro-pyran-2-ylmethoxymethyl)-pyrimidine-2,4-diamine

The titled compound was prepared according to the procedure described in Example 61A-D, substituting 2-tetrahydropyranmethanol for cyclobutanemethanol used in Example 61A.

### EXAMPLE 64B

### 5-{4-[(4-Chlorobenzyl)amino]phenyl}-6-[(tetrahydro-2H-pyran-2-ylmethoxy)methyl]pyrimidine-2,4-diamine

To a stirred solution of 5-(4-amino-phenyl)-6-(tetrahydropyran-2-ylmethoxymethyl)-pyrimidine-2,4-diamine from Example 64A (60 mg, 0.182 mmol) in MeOH (1.8 mL) was added 4-chlorobenzaldehyde (26 mg, 0.182 mmol). After 30 minutes at room temperature, the reaction was cooled to 0° C. Glacial acetic acid (0.04 mL, 0.6 mmol) was added followed by NaCNBH₃ (14 mg, 0.218 mmol). The reaction warmed to room temperature over 1 h. To the reaction was added saturated NaHCO₃ (7 mL). It was washed with EtOAc (3×7 mL). The combined organic layers were washed with brine (7 mL), dried over MgSO₄, and concentrated. The residue was triturated with isopropanol and filtered to give an off-white solid (29 mg, 35%). ¹H NMR (300 MHz, DMSO-d₆) δ 7.36-7.43 (m, 4H), 6.88 (d, J=8.48 Hz, 2H), 6.59 (d, J=8.48 Hz, 2H), 6.36 (t, J=5.93 Hz, 1H), 5.85 (s, 2H), 5.47 (s, 2H), 4.27 (d, J=5.76 Hz, 2H), 3.86 (s, 2H), 3.76 (dd, J=11.02, 2.88 Hz, 1H), 3.17-3.25 (m, 3H), 3.10-3.13 (m, 1H), 1.69-1.71 (m, 1H), 1.32-1.42 (m, 4H), 1.03-1.07 (m, 1H). MS (ESI) positive ion 454 (M+H)⁺; negative ion 452 (M−H)⁻.

### EXAMPLE 65

### 6-[(benzyloxy)methyl]-5-[4-({[6-(trifluoromethyl)pyridin-3-yl]methyl}amino)phenyl]pyrimidine-2,4-diamine

The titled compound was prepared according to the procedure described in Example 2, substituting 6-trifluolo-pyridine-3-carbaldehyde for 4-chloro-benzaldehyde. ¹H NMR (300 MHz, DMSO-d₆) δ 8.79-7.76 (m, 1H), 8.01-7.98 (m, 2H), 7.30-7.15 (m, 5H), 6.92 (d, J=8.5 Hz, 2H), 6.63 (d, J=8.5 Hz, 2H), 6.49 (t, J=6.1 Hz, 1H), 5.87 (s, 2H), 5.48 (bs, 2H), 4.45 (d, J=6.1 Hz, 2H), 4.31 (s, 2H), 3.93 (s, 2H). MS (ESI) positive ion 438 (M+H)⁺; negative ion 436 (M−H)⁻.

### EXAMPLE 66

### 4-[(4-{2,4-diamino-6-[(benzyloxy)methyl]pyrimidin-5-yl}benzyl)amino]benzonitrile

The titled compound was prepared according to the procedure described in Example 60, substituting 4-fluorobenzonitrile for 1-fluoro-4-nitrobenzene used in Example 66. ¹HNMR (DMSO-d₆, 300 MHz), δ 7.92 (m, 1H), 7.49-7.09 (m, 9H), 7.43 (d, J=8.8 Hz, 2H), 6.68 (d, J=8.8 Hz, 2H), 6.17 (s, 2H), 5.89 (s, 2H), 4.38 (d, J=6.1 Hz, 2H), 4.31 (s, 2H), 3.96 (s, 2H); MS (ESI) m/e 437 (M+H)⁺.

### EXAMPLE 67

### 3-[(4-{2,4-diamino-6-[(benzyloxy)methyl]pyrimidin-5-yl}phenoxy)methyl]benzonitrile

The titled compound was prepared by the same procedure described for Example 63, substituting 3-cyanobenzyl bromide for 4-chlorobenzyl bromide used in Example 63B. The product was purified by recrystallization from i-PrOH/H₂O or ethanol/H₂O to give 33 mg (49%) of a yellow solid. ¹H NMR (300 MHz, d₆-DMSO) δ 7.95 (s, 1H), 7.82 (m, 2H), 7.61 (t, 1H, J=7.8 Hz), 7.26 (m, 3H), 7.16 (m, 4H), 7.06 (m, 2H), 5.94 (s, 2H), 5.57 (s, 2H), 5.18 (s, 2H), 4.29 (s, 2H), 3.96 (s, 2H); MS (ESI) m/z 438 [M+H]⁺.

### EXAMPLE 68

### 5-{[(4-{2,4-diamino-6-[(benzyloxy)methyl]pyrimidin-5-yl}phenyl)amino]methyl}pyridine-2-carbonitrile

The titled compound was prepared according to the procedure described in Example 2, substituting 6-cyano-pyridine-3-carbaldehyde (Ashimori, Atsuyuki et al.; Chem.Pharm.Bull.; EN; 38; 9; 1990; 2446-2458) for 4-chloro-benzaldehyde. ¹H NMR (300 MHz, DMSO-d₆) δ 8.45 (d, J=2.4 Hz, 1H), 7.85 (dd, J=8.5 Hz, J=2.4 Hz, 1H), 7.49 (d, J=8.5 Hz, 1H), 7.28-7.15 (m, 5H), 6.92 (d, J=8.8 Hz, 2H), 6.63 (d, J=8.8 Hz, 2H), 6.39 (t, J=6.1 Hz, 1H), 5.87 (s, 2H), 5.50 (bs, 2H), 4.32, 4.33 (s, s, 4H), 3.93 (s, 2H). MS(ESI) positive ion 447 (M+H)⁺; negative ion 445 (M−H)⁻.

### EXAMPLE 69

### 6-[(benzyloxy)methyl]-5-{4-[2-(4-chlorophenyl)ethoxy]phenyl}pyrimidine-2,4-diamine

The titled compound was prepared by the same procedure described for Example 63, substituting 4-chlorophenethyl bromide (Saunders, W. H. Jr.; Williams, R. A. J. Am. Chem. Soc. 1957, 79, 3712) for 4-chlorobenzyl bromide used in Example 63B. The product was purified by reverse phase HPLC to give 17 mg (19%) of a yellow solid. ¹H NMR (300 MHz, d₆-DMSO) δ 11.69 (s, 1H), 8.24 (s, 1H), 7.62 (bs, 2H), 7.38 (s, 4H), 7.27 (m, 5H), 7.12 (m, 2H), 7.03 (m, 2H), 6.90 (m, 1H), 4.47 (s, 2H), 4.22 (t, 2H, J=6.4 Hz), 4.15 (s, 2H), 3.06 (t, 2H, J=6.4 Hz); MS (ESI) m/z 461 [M+H]⁺.

### EXAMPLE 70

### 6-[(benzyloxy)methyl]-5-[4-(pyridin-3-ylmethoxy)phenyl]pyrimidine-2,4-diamine

The titled compound was prepared by the same procedure described for Example 63, substituting 3-(chloromethyl)pyridine hydrochloride for 4-chlorobenzyl bromide used in Example 63B and adding an additional 0.15 mmol potassium ethoxide. Heating at reflux was required to complete the substitution reaction The product was purified by HPLC to give 21 mg (22%) of the bis[trifluoroacetate] salt as a foam. ¹H NMR (300 MHz, d₆-DMSO) δ 11.65 (s, 1H), 8.77 (d, 1H, J=1.4 Hz), 8.61 (dd, 1H, J=4.7, 1.7 Hz), 8.27 (s, 1H), 8.00 (ddd, 1H, J=7.8, 2.0, 1.7 Hz), 7.59 (bs, 2H), 7.54 (ddd, J=7.8, 5.1, 0.7 Hz), 7.32 (m, 6H), 7.15 (m, 4H), 6.95 (s, 1H), 5.22 (s, 2H), 4.52 (s, 2H), 4.17 (s, 2H); MS (ESI) m/z 414 [M+H]⁺.

### EXAMPLE 71

### 6-[(benzyloxy)methyl]-5-{4-[(tetrahydro-2H-pyran-4-ylmethyl)amino]phenyl}pyrimidine-2,4-diamine

### EXAMPLE 77A

### 4-(Hydroxymethyl)tetrahydropyran

To an ice-cooled solution of 2.60 g (20.0 mmol) of tetrahydropyran-4-carboxylic acid in 8 mL of THF was added 21 mL (21 mmol) of 1.0M borane in THF. The reaction was stirred at 0 C for 1 h, then quenched by dropwise addition of 2 mL of water. After stirring for 10 min at ambient temperature, solid K₂CO₃ was added and swirled until free flowing. The salts were filtered, and the supernatant was concentrated to 1.25 g (54%) of 4-(hydroxymethyl)tetrahydropyran as a colorless oil.

### EXAMPLE 77B

### 4-Tetrahydropyran carboxaldehyde

To a solution of 116 mg (1.00 mmol) of 4-(hydroxymethyl)tetrahydropyran from Example 77A in 2 mL of CH₂Cl₂ was added 424 mg (1.0 mmol) of the Dess-Martin periodinane. The mixture was stirred at ambient temperature for 1 h, then filtered through diatomaceous earth. The filter cake was washed with about 3 mL of CH₂Cl₂, then the tilted aldehyde solution was used directly in the next step.

### EXAMPLE 77C

### 6-[(Benzyloxy)methyl]-5-{4-[(tetrahydro-2H-pyran-4-ylmethyl)amino]phenyl}pyrimidine-2,4-diamine

To a solution of 1 mmol of 4-tetrahydropyran carboxaldehyde in about 5 mL of CH₂Cl₂ was added 200 mg of 5-(4-amino-phenyl)-6-benzyloxymethyl-pyrimidine-2,4-diamine. The solution was stirred for 10 min, then concentrated in vacuo. The residue was dissolved in 2 mL of methanol and 0.4 mL of glacial acetic acid. To the solution was added 100 mg (1.59 mmol) of sodium cyanoborohydride. The reaction was stirred at ambient temperature for 1 h, then concentrated in vauco. The residue was dissolved in 5 mL of 2M NaOH_{(aq.)} and extracted with ethyl acetate (2×5 mL). The combined organic layers were back extracted with 2M NaOH (1×5 mL), and brine (1×5 mL), dried over MgSO₄, filtered, and concentrated to 222 mg of a foam. A 99 mg portion of this crude product was purified by reverse phase HPLC, eluting with 5% to 100% CH₃CN in aq 0.1% trifluoroacetic acid to give 48 mg (27%) of the product as its bis(trifluoroacetate) salt. ¹H NMR (300 MHz, d₆-DMSO) δ 11.51 (s, 1H), 8.27 (s, 1H), 7.53 (bs, 2H), 7.32 (m, 6H), 6.90 (d, 2H, J=8.5 Hz), 6.85 (s, 1H), 6.62 (d, 2H, J=8.8 Hz), 4.48 (s, 2H), 4.20 (s, 2H), 3.95 (dd, 2H, J=11.5, 2.7 Hz), 3.28 (td, 2H, J=11.7, 2.0 Hz), d.92 (d, 2H, J=6.4 Hz), 1.79 (m, 1H), 1.69 (m, 2H), 1.27 (dd, 1H, J=11.9, 4.4 Hz), 1.19 (dd, 1H, J=11.9, 3.7 Hz); MS (ESI) m/z 420 [M+H]⁺.

### EXAMPLE 72

### 6-[(benzyloxy)methyl}-5-(4-{[4-(trifluoromethoxy)benzyl]amino}phenyl)pyrimidine-2,4-diamine

The titled compound was prepared according to the procedure described in Example 2, substituting 4-trifluoromethoxy-benzaldehyde for 4-chloro-benzaldehyde. ¹H NMR (300 MHz, DMSO-d₆) δ 7.52 (d, J=8.5 Hz, 2H), 7.34-7.15 (m, 7H), 6.91 (d, J=8.5 Hz, 2H), 6.62 (d, J=8.5 Hz, 2H), 6.38 (t, J=5.8 Hz, 1H), 5.87 (s, 2H), 5.47 (bs, 2H), 4.34-4.30 (m, 4H), 3.94 (s, 2H); MS (ESI) positive ion 496 (M+H)⁺; negative ion 494 (M−H)⁻.

### EXAMPLE 73

### 4-{[(4-{2,4-diamino-6-[(benzyloxy)methyl]pyrimidin-5-yl}phenyl)amino]methyl}pyridine-2-carbonitrile

The titled compound was prepared according to the procedure described in Example 2, substituting 2-cyano-pyridine-4-carbaldehyde (Ashimori, Atsuyuki et al.; Chem.Pharm.Bull.; EN; 38; 9; 1990; 2446-2458) for 4-chloro-benzaldehyde. ¹H NMR (300 MHz, DMSO-d₆) δ 8.68 (d, J=5.1 Hz, 1H), 8.0 (s, 1H), 7.72 (d, J=5.1 Hz, 1H), 7.30-7.15 (m, 5H), 6.92 (d, J=8.8 Hz, 2H), 6.60 (d, J=8.8 Hz, 2H), 6.52 (t, J=6.1 Hz, 1H), 5.88 (s, 2H), 5.52 (bs, 2H), 4.42 (d, J=6.1 Hz, 2H), 4.31 (s, 2H), 3.93 (s, 2H). MS (ESI) positive ion 438 (M+H)⁺; negative ion 436 (M−H)⁻.

### EXAMPLE 74

### 6-[(4-{2,4-diamino-6-[(benzyloxy)methyl]pyrimidin-5-yl}benzyl)amino]nicotinonitrile

The titled compound was prepared according to the procedure described in Example 60, substituting 6-chloronictonitrile for 1-fluoro-4-nitrobenzene used in Example 60. ¹H NMR (DMSO-d₆, 300 MHz), δ 8.42-8.36 (m, 1H), 8.17-8.11 (m, 1H), 7.37-7.12 (m, 8H), 6.64 (d, J=8.8 Hz, 2H), 5.97 (s, 2H), 5.57 (s, 2H), 4.60 (d, J=6.1 Hz, 2H), 4.31 (s, 2H), 3.94 (s, 2H); MS (ESI) m/e 438 (M+H)⁺.

### EXAMPLE 75

### 5-{4-[(4-chlorobenzyl)amino]phenyl}-6-{[(3-chlorobenzyl)oxy]methyl}pyrimidine-2,4-diamine

To a solution of {2,6-diamino-5-[4-(4-chloro-benzylamino)-phenyl]-pyrimidin-4-yl}-methanol (51.2 mg, 0.14 mmol) in anhydrous DMF (0.5 mL) was added 3-chlorobenzylbromide (16.5 mL, 0.126 mmol). The reaction was stirred a few minutes and then sodium t-butoxide was added (14.6 mg, 0.154 mmol) and stirred for 24 h. Dilute reaction mixture to 2 mL with methanol and purify by preperative HPLC (5-100% CH₃CN/0.1% TFA in H₂O, Synergi Hydro-RP by Phenomenex). The desired fractions were concentrated in vaccuo to yield 22.3 mg (22%) of white solid. ¹H NMR (300 MHz, DMSO-d₆) δ 11.53 (s, 1H), 8.25 (br s, 1H), 7.41 (s, 4H), 7.34-7.37 (m, 3H), 7.25-7.20 (m, 1H), 6.91 (s, 1H), 6.88 (s, 1H), 6.65 (s, 1H), 4.47 (s, 2H), 4.28 (d, 2H), 4.18 (s, 2H). MS (DCI/NH₃): 480, 482, 484 (M+H)⁺.

### EXAMPLE 76

### 5-{4-[(4-chlorobenzyl)amino]phenyl}-6-{[(2-methylbenzyl)oxy]methyl}pyrimidine-2,4-diamine

The titled compound was prepared according to the procedure described in Example 82, substituting 2-methylbenzylbromide (16.5 mL, 0.126 mmol) for 3-chlorobenzylbromide (16.5 mL, 0.126 mmol) to yield 29.0 mg (29%) of the titled compound as a white solid. ¹H NMR (300 MHz, DMSO-d₆) δ 11.51 (s, 1H), 8.25 (br s, 1H), 7.41 (s, 4H), 7.09-7.20 (m, 4H), 6.93 (s, 1H), 6.90 (s, 1H), 6.65 (s, 1H), 6.62 (s, 1H), 4.44 (s, 2H), 4.29 (d, 2H), 4.17 (s, 2H), 2.22 (s, 3H). MS (DCI/NH₃) m/e 460, 462 (M+H)⁺.

### EXAMPLE 77

### 5-{4-[(4-chlorobenzyl)amino]phenyl}-6-[(cyclohexylmethoxy)methyl]pyrimidine-2,4-diamine

### EXAMPLE 77A

### 5-(4-Amino-phenyl)-6-cyclohexylmethoxymethyl-pyrimidine-2,4-diamine

The titled compound was prepared according to the procedure described in Example 61A-D, substituting cyclohexanemethanol for cyclobutanemethanol used in Example 61A.

### EXAMPLE 77B

### 5-{4-[(4-Chlorobenzyl)amino]phenyl}-6-[(cyclohexylmethoxy)methyl]pyrimidine-2,4-diamine

5-(4-Amino-phenyl)-6-cyclohexylmethoxymethyl-pyrimidine-2,4-diamine from Example 77A (50 mg, 0.14 mmol) was combined with 10% Pd/C (5 mg), taken up in glacial acetic acid (1.4 mL) and placed under an atmosphere of H₂. The reactionwas complete after 4 h at room temperature. It was filtered through Celite, rinsed with MeOH and concentrated. The residue was dissolved in MeOH (1.4 mL) and 4-chlorobenzaldehyde (20 mg, 0.14 mmol) was added. The reaction is cooled to 0° C. and glacial acetic acid (0.03 mL) and NaCNBH₃ (11 mg, 0.17 mmol) were added. The reaction warmed to room temperature and was complete after 1.0 h. Saturated NaHCO₃ (5 mL) and the aqueous is extracted with EtOAc (2×10 mL). The combined organic layers were washed with brine, dried over MgSO4 and concentrated. The resulting residue was purified by reverse-phase HPLC (0-70% CH₃CN, aqueous NH₄OAc) to give a white powder (6 mg, 10%). ¹H NMR (300 MHz, DMSO-d₆) δ 7.36-7.42 (m, 4H), 6.88 (d, J=8.48 Hz, 2H), 6.58 (d, J=8.48 Hz, 2H), 6.36 (t, J=5.93 Hz, 1H), 5.86 (s, 2H), 5.50 (s, 2H), 4.27 (d, J=5.76 Hz, 2H), 3.80 (s, 2H), 3.11-3.24 (m, 1H), 2.99 (d, J=6.44 Hz, 2H), 1.54-1.58 (m, 4H), 1.23-1.35 (m, 2H), 1.08-1.14 (m, 2H), 0.76-0.90 (m, 2H). MS (ESI) positive ion 452 (M+H)⁺; negative ion 450 (M−H)⁻.

### EXAMPLE 78

### 6-[(benzyloxy)methyl]-5-{4-[(4-nitrobenzyl)amino]phenyl}pyrimidine-2,4-diamine

The title compound was synthesized according to the procedure described in Example 2, substituting 4-nitrobenzaldehyde for 4-chlorobenzaldehyde. ¹H NMR (300 MHz, DMSO-d₆) δ 3.95 (s, 2H), 4.32 (s, 2H), 4.44 (d, J=6.10 Hz, 2H), 5.61 (s, 2H), 5.97 (s, 2 H), 6.55 (t, J=6.27 Hz, 1 H), 6.60 (d, J=8.48 Hz, 2 H), 6.90 (d, J=8.48 Hz, 2 H), 7.16 (dd, J=7.46, 2.03 Hz, 2 H), 7.25 (m, 3 H), 7.66 (d, J=8.81 Hz, 2 H), and 8.20 (ddd, J=9.07, 2.54, 2.29 Hz, 2 H); MS (ESI) positive ion 457 (M+H)⁺; negative ion 455 (M−H)⁻.

### EXAMPLE 79

### 6-ethyl-5-{4-[(4-nitrobenzyl)amino]phenyl}pyrimidine-2,4-diamine

The titled compound was prepared according to the procedure described in Example 1, substituting 4-nitro-benzaldehyde for 4-chloro-benzaldehyde. ¹H NMR (300 MHz, DMSO-d₆) δ 8.22 (d, J=8.8 Hz, 2H), 7.67 (d, J=8.8 Hz, 2H), 6.86 (d, J=8.5 Hz, 2H), 6.62 (d, J=8.5 Hz, 2H), 6.51 (t, J=6.1 Hz, 1H), 5.71 (s, 2H), 5.23 (bs, 2H), 4.44 (d, J=6.1 Hz, 2H), 2.10 (q, J=7.5 Hz, 2H), 0.94 (t, J=7.5 Hz, 3H). MS (ESI) positive ion 365 (M+H)⁺.

### EXAMPLE 80

### 6-[(benzyloxy)methyl]-5-(4-{[(2-chloropyridin-4-yl)methyl]amino}phenyl)pyrimidine-2,4-diamine

The titled compound was prepared according to the procedure described in Example 2, substituting 2-chloro-pyridine-4-carbaldehyde (Watson, Samuel E. et al.; Heterocycles; EN; 48; 10; 1998; 2149-2156) for 4-chloro-benzaldehyde. ¹H NMR (300 MHz, DMSO-d₆) δ 8.34 (d, J=5.1 Hz, 1H), 7.49 (s, 1H), 7.42 (d, J=5.1 Hz, 1H), 7.30-7.15 (m, 5H), 6.91 (d, J=8.5 Hz, 2H), 6.60 (d, J=8.5 Hz, 2H), 6.49 (t, J=6.5 Hz, 1H), 5.87 (s, 2H), 5.50 (bs, 2H), 4.36 (d, J=6.5 Hz, 2H), 4.31 (s, 2H), 3.93 (s, 2H). MS (ESI) positive ion 447 (M+H)⁺; negative ion 445 (M−H)⁻.

### EXAMPLE 81

### 6-[(benzyloxy)methyl]-5-{4-[(pyrimidin-5-ylmethyl)amino]phenyl}pyrimidine-2,4-diamine

### EXAMPLE 81A

### Pyrimidine-5-carboxaldehyde:

A modified procedure of Rho and Abuh (Syn. Commun. 1994, 24, 253-256) was followed for the preparation of the titled aldehyde. Under nitrogen, to a solution of 5-bromopyrimidine (1 g, 6.3 mmol) in 60 mL anhydrous THF, was added BuLi (2.5 M, 2.6 mL, 6.5 mmol) at −78° C. The resulting yellow solution was stirred for 20 min, after which ethyl formate (0.55 mL, 6.7 mmol) was added dropwise over 5 min. After 20 min, the reaction was quenched with 1.5 M THF/HCl solution (4.5 mL, 6.7 mmol). The cold bathwas removed, and the reaction mixture was stirred for 1 h. THF was removed in vacco, 10 mL of water was then added. The mixture was extracted with CHCl₃ (2×10 mL), and the combined organics were dried (MgSO₄) and concentrated. The crude product was purified via flash column chromatography (5% MeOH/CHCl₃) to give 0.35 g (51%) of the titled pyrimidine-5-carboxaldehyde.

### EXAMPLE 81B

### 6-[(Benzyloxy)methyl]-5-{4-[(pyrimidin-5-ylmethyl)amino]phenyl}pyrimidine-2,4-diamine

The titled compound was then prepared according to the procedure described in Example 2, substituting pyrimidine-5-carboxaldehyde from Example 87A for 4-chlorobenzaldehyde used in Example 2. ¹H NMR (300 Hz, DMSO-d₆) δ 9.08 (s, 1H), 8.82 (s, 2H), 7.27-7.16 (m, 5H), 6.93 (d, J=9 Hz, 2H), 6.66 (d, J=6 Hz, 2H), 6.39 (t, J=6 Hz, 1H), 5.87 (s, 2H), 5.50 (s, 2H), 4.35 (d, J=6 Hz, 2H), 4.32 (s, 2H), 3.94 (s, 2H). MS(ESI) positive ion 414 (M+H)⁺; negative ion 412 (M−H)⁻.

### EXAMPLE 82

### 6-[(benzyloxy)methyl]-5-{4-[(thien-2-ylmethyl)amino]phenyl}pyrimidine-2,4-diamine

The titled compound was then prepared according to the procedure described in Example 2, substituting thiophene-2-carboxaldehyde for 4-chlorobenzaldehyde used in Example 2. ¹H NMR (300 Hz, DMSO-d₆) δ 7.38 (d, J=6 Hz, 1H), 7.31-7.17 (m, 5H), 7.20 (d, J=3 Hz, 1H), 6.98 (d, J=3 Hz, 1H), 6.92 (d, J=9 Hz, 2H), 6.68 (d, J=9 Hz, 2H), 6.33 (t, J=6 Hz, 1H), 5.86 (s, 2H), 5.48 (s, 2H), 4.46 (d, J=6 Hz, 2H), 4.33 (s, 2H), 3.95 (s, 2H). MS(ESI) positive ion 418 (M+H)⁺; negative ion 416 (M−H)⁻.

### EXAMPLE 83

### 6-[(benzyloxy)methyl]-5-{4-[(thien-3-ylmethyl)amino]phenyl}pyrimidine-2,4-diamine

The titled compound was then prepared according to the procedure described in Example 2, substituting thiophene-3-carboxaldehyde for 4-chlorobenzaldehyde used in Example 2. ¹H NMR (300 Hz, DMSO-D₆) δ 7.49 (d, J=6 Hz, 1H), 7.38 (d, J=3 Hz, 1H), 7.31-7.18 (m, 5H), 7.13 (d, J=6 Hz, 1H), 6.91 (d, J=9 Hz, 2H), 6.65 (d, J=6 Hz, 2H), 6.17 (t, J=6 Hz, 1H), 5.86 (s, 2H), 5.49 (s, 2H), 4.33 (s, 2H), 4.26 (d, J=6 Hz, 2H), 3.96 (s, 2H). MS(ESI) positive ion 418 (M+H)⁺; negative ion 416 (M−H)⁻.

### EXAMPLE 84

### 6-[(benzyloxy)methyl]-5-[4-({[1-(4-chlorophenyl)ethyl]amino}methyl)phenyl]pyrimidine-2,4-diamine

To 5-(4-aminomethyl-phenyl)-6-benzyloxymethyl-pyrimidine-2,4-diamine from Example 58B (33.5 mg, 0.1 mmol) ) in methanol (2 ml) and a buffer solution of acetic acid and sodium acetate (1 ml, pH 4-5) was added 4-chloroacetophenone (18.5 mg, 0.12 mmol), then NaBH₃CN (76 mg, 0.12 mmol). The reaction mixture was stirred at r.tfor 2 h before the solvents were removed on evaporator under pressure. The residue was purified by column chromatography to yield the titled compound (29 mg, 61%). ¹H NMR (300 MHz, DMSO-d₆) δ 7.38 (s, 5H), 7.32-7.13 (m, 8H), 5.97 (s, 2H), 5.56(s, 2H), 4.32(s, 2H), 3.96 (s, 2H), 3.73 (q, J=6.0 Hz, 1H), 3.52 (d, J=3.0 Hz, 2H), 1.26(d, 3H). MS (ESI) positive ion 474 (M+H)⁺; negative ion 472 (M−H)⁻.

### EXAMPLE 85

### 6-[(benzyloxy)methyl]-5-(4-{[2-(4-nitrophenyl)ethyl]amino}phenyl)pyrimidine-2,4-diamine

The titled compound was prepared according to the procedure described in Example 2, substituting (4-nitro-phenyl)-acetaldehyde (Ashwell, Mark A. et al, Bioorg.Med.Chem.Lett.; EN; 11; 24; 2001; 3123-3128) for 4-chloro-benzaldehyde. ¹H NMR (300 MHz, DMSO-d₆) δ 8.18 (d, J=8.8 Hz, 2H), 7.60 (d, J=8.8 Hz, 2H), 7.32-7.18 (m, 5H), 6.93 (d, J=8.5 Hz, 2H), 6.64 (d, J=8.5 Hz, 2H), 5.87 (s, 2H), 5.81 (t, J=6.1 Hz, 1H), 5.47 (bs, 2H), 4.35 (s, 2H), 3.97 (s, 2H), 3.38-3.22 (m, 2H), 3.01 (t, J=7.1 Hz, 2H). MS (ESI) positive ion 471 (M+H)⁺.

### EXAMPLE 86

### 6-[(benzyloxy)methyl]-5-(4-{[2-(4-chlorophenyl)ethyl]amino}phenyl)pyrimidine-2,4-diamine

The titled compound was prepared according to the procedure described in Example 2, substituting (4-chloro-phenyl)-acetaldehyde for 4-chloro-benzaldehyde. ¹H NMR (300 MHz, DMSO-d₆) δ 7.38-7.18 (m, 5H), 6.93 (d, J=8.5 Hz, 2H), 6.63 (d, J=8.5 Hz, 2H), 5.87 (s, 2H), 5.76 (t, J=6.1 Hz, 1H), 5.51 (bs, 2H), 4.35 (s, 2H), 3.97 (s, 2H), 3.38-3.22 (m, 2H), 2.85 (t, J=7.1 Hz, 2H). MS (ESI) positive ion 460 (M+H)⁺; negative ion 458 (M−H)⁻.

### EXAMPLE 87

### 6-[(benzyloxy)methyl]-5-{4-[(cycloheptylamino)methyl]phenyl}pyrimidine-2,4-diamine

The titled compound was prepared according to the procedure described in Example 58, substituting cycloheptanone for 4-chlorobenzaldehyde used in Example 58 (78% yield). ¹H NMR (300 MHz, DMSO-D₆) δ 7.37-7.14 (m, 9H), 5.96 (s, 2H), 5.56(s, 2H), 4.31 (s, 2H), 3.96 (s, 2H), 3.73 (s, 2H), 2.64 (m, 1H), 1.88-1.33(m, 13H). MS (ESI) positive ion 432 (M+H)⁺; negative ion 430 (M−H)⁻.

### EXAMPLE 88

### 6-[(benzyloxy)methyl]-5-[4-pyridin-4-ylmethoxy)phenyl]-pyrimidine-2,4-diamine

The titled compound was prepared by the same procedure described for Example 63, substituting 4-(chloromethyl)pyridine hydrochloride for 4-chlorobenzyl bromide used in Example 63B, and adding an additional 0.15 mmol potassium ethoxide. Heating at reflux was required to complete the substitution reaction. The product was purified by recrystallization from i-PrOH/H₂O or ethanol/H₂O to give 8 mg (13%) of a solid. ¹H NMR (300 MHz, d₆-DMSO) δ 8.59 (d, 2H, J=6.1 Hz), 7.48 (d, 2H, J=5.8 Hz), 7.27 (m, 3H), 7.15 (m, 4H), 7.04 (d, 2H, J=8.6 Hz), 5.94 (s, 2H), 5.59 (s, 2H), 5.15 (s, 2H), 4.26 (s, 2H), 3.88 (s, 2H); MS (ESI) m/z 414 [M+H]⁺.

### EXAMPLE 89

### 6-{[(3-Methylbut-2-enyl)oxyl]methyl}-5-(4-{[4-(methylsulfonyl)benzyl]amino}phenyl)pyrimidine-2,4-diamine

To a solution of NaH (20 mg, 0.56 mmol) in anhydrous THF (1.7 mL) was added 3-methyl-2-buten-1-ol (0.07 mL, 0.69 mmol). After 20 minutes at room temperature, 6-bromomethyl-5-[4-(4-methanesulfonyl-benzylamino)-phenyl]-pyrimidine-2,4-diamine from Example 98A (80 mg, 0.17 mmol) in a solution of THF (0.5 mL) and DMPU (0.2 mL) was added. The reaction was complete after 40 minutes at room temperature. It was diluted with H₂O (5 mL) and extracted with EtOAc (3×5 mL). The combined organic layers were washed with H₂O (5 mL), brine (5 mL), dried over MgSO₄ and concentrated. Purification on reverse-phase HPLC (0-70% CH₃CN, aqueous NH₄OAc) and trituration with Et₂O provided a white solid (10 mg, 12% yield). ¹H NMR (300 MHz, DMSO-d₆) δ 7.87-7.90 (d, J=8.47 Hz, 2H), 7.63-7.66 (d, J=8.14 Hz, 2H), 6.87-6.90 (d, J=8.48 Hz, 2H), 6.60-6.62 (d, J=8.47 Hz, 2H), 6.49-6.54 (t, J=6.10 Hz, 1 H), 5.86 (s, 2H), 5.47 (s, 2H), 5.08-5.13 (t, J=6.78 Hz, 1 H), 4.39-4.41 (d, J=6.10 Hz, 2H), 3.82 (s, 2H), 3.74-3.76 (d, J=6.44 Hz, 2H), 3.19 (s, 3H), 1.62 (s, 3H), 1.51 (s, 3H). MS (ESI) positive ion 468(M+H)⁺; negative ion 466 (M−H)⁻.

### EXAMPLE 90

### 3-[2,6-diamino-5-(4-{[4-(methylsulfonyl)benzyl]amino}phenyl)pyrimidin-4-yl]-N-(3,5-dimethylbenzyl)propanamide

The titled compound was prepared according to procedure in Example 57C, substituting 3,5-dimethylbenzylamine for n-butylamine and 4-methylsufonylbenzaldehyde for 4-chlorobenzaldehyde. The crude product was purified via column chromatography eluting with 95% CH₂Cl₂/MeOH (0.5% NH₄OH) to give a yellow solid (10 mg, 14% yield). ¹H NMR (300 MHz, DMSO-d₆) δ 8.16-8.20 (t, J=5.76 Hz, 6.11 Hz, 1H), 7.88-7.90 (d, 8.47 Hz, 2H), 7.64-7.66 (d, J=8.14 Hz, 2H), 6.86-6.89 (d, J=8.48 Hz, 2H), 6.84 (s, 2H), 6.77 (s, 2H), 6.61-6.63 (d, J=8.47 Hz, 2H), 6.46-6.60 (t, J=6.11 Hz, 5.76 Hz, 1H), 5.70 (s, 2H), 5.31 (s, 2H), 4.39-4.41 (d, 5.76 Hz, 2H), 4.11-4.13 (d, J=5.77 Hz, 2H), 3.19 (s, 3H), 2.36 (s, 3H), 2.22 (s, 5H). MS (ESI) positive ion 559 (M+H)⁺; negative ion 557 (M−H)⁻.

### EXAMPLE 91

### 6-[(benzyloxy)methyl]-5-(4-{[3-fluoro-4-(methylsulfonyl)benzyl]amino}phenyl)pyrimidine-2,4-diamine

### EXAMPLE 91A

### 3-Fluoro-4-methanesulfonyl-benzaldehyde

A mixture of 3,4-difluorobenzaldehyde (250 mg, 1.76 mmol), sodium methanesulfinate (198 mg, 1.94 mmol) in 2.0 mL of anhydrous DMSO was flushed with N₂, and heated in a microwave reactor for 20 min at 185° C. Water (5 mL) was added to the reaction mixture, which was then extracted with EtOAc (10 mL). The organic layer was washed with water and brine, dried over Na₂SO₄, and evaporated in vacuuo to give the titled compound (200 mg, 70% yield), which was used directly without any further purification.

### EXAMPLE 91B

### 6-[(benzyloxy)methyl]-5-(4-{[3-fluoro-4-(methylsulfonyl)benzyl]amino}phenyl)pyrimidine-2,4-diamine

The aniline from Example 2C (56 mg, 0.17 mmol) was dissolved in MeOH buffer (1 M, NaOAc/HOAC, pH4, 1.5 mL) followed by addition of 3-fluoro-4-methanesulfonyl-benzaldehyde (35 mg, 0.17 mmol) from Example 91A and NaBH₃CN (13 mg, 0.21 mmol). After 20 min, the reaction mixture was quenched with conc. HCl (aq, 3 drops) and purified by HPLC to give the titled compound (43 mg, 49%). ¹H NMR (300 MHz, DMSO-d₆) δ 7.83 (t, 1H, J=7.8 Hz), 7.50 (dd, J=7.8, 1.4 Hz, 1H), 7.47 (dd, J=4.4, 1.4 Hz, 1H), 7.30-7.22 (m, 3H), 7.16 (dd, J=7.8, 2.3 Hz, 2H), 6.91 (d, J=8.8 Hz, 2H), 6.60 (d, J=8.8 Hz, 2H), 6.52 (t, J=5.9 Hz, 1H), 5.87 (s, 2H), 5.49 (s, 2H), 4.42 (d, J=6.1 Hz, 2H), 4.32 (s, 2H), 3.94 (s, 2H), 3.29 (s, 3H); MS (ESI) m/e 508 (M+H)⁺.

### EXAMPLE 92

### 5-(3-amino-4-{[4-(methylsulfonyl)benzyl]amino}phenyl)-6-ethylpyrimidine-2,4-diamine

### EXAMPLE 92A

### 6-Ethyl-5-[4-(4-methanesulfonyl-benzylamino)-3-nitro-phenyl]-pyrimidine-2,4-diamine

The mixture of 5-(4-Chloro-3-nitro-phenyl)-6-ethyl-pyrimidine-2,4-diamine (235 mg, 0.8 mmol), 4-methanesulfonylbenzylamine (355 mg, 1.6 mmol) and N,N′-diisopropylethylamine in NMP was heated in a microwave oven at 190° C. for 10 minuntes. It was then partitioned between ethyl acetate and aqueous NaHCO₃ (50 mL, 1:1). The organic phase was washed with brine, dried (MgSO₄), filtered and concentrated under reduced pressure. Purification by Reverse Phase Chromatography provided the titled compound (52 mg).

### EXAMPLE 92B

### 5-[3-Amino-4-(4-methanesulfonyl-benzylamino)-phenyl]-6-ethyl-pyrimidine-2,4-diamine

The suspension of Example 92A (44 mg, 0.1 mmol) in ethanol (2 mL) was heated to 60° C. SnCl₂.2 H₂O (113 mg, 0.5 mmol) was added. The reaction mixture was heated to reflux for 1 hr. It was then partitioned between ethyl acetate and aqueous NaHCO₃ (20 mL, 1:1). The organic phase was washed with brine, dried (MgSO₄), filtered and concentrated under reduced pressure. The residue was triturated with acetonitrile to provide the titled compound (25 mg). ¹H NMR (300 MHz, DMSO-d₆) δ 7.90 (d, 2H, J=8.5 Hz), 7.67 (d, 2H, J=8.5 Hz), 6.40 (d, 1H, J=2.0 Hz), 6.35 (d, 1H, J=8.1 Hz), 6.22 (dd, 1H, J=2.0 Hz, J=8.1 Hz), 5.68 (s, 2H), 5.33 (t, 1H, J=5.4 Hz), 5.22 (bs, 2H), 4.67 (s, 2H), 4.43 (d, 2H, J=5.4 Hz), 3.20 (s, 3H), 2.13 (q, 2H, J=7.5 Hz), 0.95 (t, 3H, J=7.5 Hz); MS (ESI) positive ion 413 (M+H)⁺; negative ion 411 (M−H)⁻.

### EXAMPLE 93

### 1-[4-({[4-(2,4-diamino-6-{[(3-methylbut-2-enyl)oxy]methyl}pyrimidin-5-yl)phenyl]amino}methyl)phenyl]ethanone

### EXAMPLE 93A

### 5-(4-Amino-phenyl)-6-bromomethyl-pyrimidine-2,4-diamine acetic acid salt

[2,6-Diamino-5-(4-amino-phenyl)-pyrimidin-4-yl]-methanol from Example 3A (200 mg, 0.864 mmol) was dissolved in 30% HBr in acetic acid (2.5 mL) and H₂O (0.1 mL), sealed in a pressure tube and heated at 120° C. for 1.25 h. It was cooled to room temperature and concentrated under reduced pressure. An orange solid was isolated (200 mg, 66% yield) as the titled compound.

### EXAMPLE 93B

### 5-(4-Amino-phenyl)-6-(3-methyl-but-2-enyloxymethyl)-pyrimidine-2,4-diamine

To a solution of 5-(4-amino-phenyl)-6-bromomethyl-pyrimidine-2,4-diamine acetic acid salt from Example 93A (80 mg, 0.272 mmol) in DMSO (1.9 mL) was added 0.82 mL of 2.0 M solution of sodium 3-methyl-2-butene-1-oxide (generated in situ with Na metal and 3-methyl-2-butene-1-ol). The reaction was quenched with H₂O (10 mL) after 30 minutes at room temperature. It was extracted with EtOAc (3×10 mL). The combined organic layers were washed with H₂O (10 mL) and brine (10 mL), dried over MgSO₄ and concentrated. Purified via column chromatography, eluting with 95% CH₂Cl₂MeOH (0.5% NH₄OH) to give the titled compound as a white solid (15 mg, 24% yield).

### EXAMPLE 93C

### 1-[4-({[4-(2,4-diamino-6-{[(3-methylbut-2-enyl)oxy]methyl}pyrimidin-5-yl)phenyl]amino}methyl)phenyl]ethanone

The titled compound was prepared according to procedure in Example 1D, substituting 5-(4-Amino-phenyl)-6-(3-methyl-but-2-enyloxymethyl)-pyrimidine-2,4-diamine (13 mg, 0.43 mmol) from Example 93B for 5-(4-amino-phenyl)-6-ethyl-pyrimidine-2,4-diamine and 4-acetyl benzaldehyde (7 mg, 0.43 mmol) for 4-chlorobenzaldehyde. The final product was purified by reverse HPLC (5-100% aqueous NH₄OAc/CH₃CN) to give an off-white solid (5 mg, 27% yield). ¹H NMR (300 MHz, DMSO-d₆) δ 7.91-7.94 (d, J=8.14 Hz, 2H), 7.51-7.54 (d, J=8.14 Hz, 2H), 6.86-6.89 (d, J=8.48 Hz, 2H), 6.59-6.63 (d, J=8.48 Hz, 2H), 6.43-6.47 (t, J=6.10 Hz, 1H), 5.84 (s, 2H), 5.45 (s, 2H), 5.08-5.12 (t, J=6.78 Hz, 6.44 Hz, 1H), 4.36-4.48 (d, J=6.10 Hz, 2H), 3.82 (s, 2H), 3.73-3.76 (d, J=6.78 Hz, 2H), 2.65 (s, 3H), 1.62 (s, 3H), 1.51 (s, 3H). MS (ESI) positive ion 432 (M+H)⁺; negative ion 430 (M−H)⁻.

### EXAMPLE 94

### 6-[(3-methylbutoxy)methyl]-5-(4-{[4-(methylsulfonyl)benzyl]amino}phenyl)pyrimidine-2,4-diamine

The titled compound was prepared according to procedure in Example 91B, substituting 5-(4-amino-phenyl)-6-(3-methyl-butoxymethyl)-pyrimidine-2,4-diamine from Example 45E for 5-(4-amino-phenyl)-6-benzyloxymethyl-pyrimidine-2,4-diamine used in Example 91B and 4-methanesulfonyl-benzaldehyde for 3-fluoro-4-methanesulfonyl-benzaldehyde used in Example 91B. ¹H NMR (300 MHz, DMSO-d₆) δ 7.89 (d, J=8.1 Hz, 2H), 7.64 (d, J=8.5 Hz, 2H), 6.89 (d, J=8.5 Hz, 2H), 6.61 (d, J=8.8 Hz, 2H), 6.52 (t, J=5.9 Hz, 1H), 6.07 (s, 2H), 6.03 (s, 2H), 4.40 (s, J=5.8 Hz, 2H), 3.86 (s, 2H), 3.23 (t, J=6.6 Hz, 2H), 3.19 (s, 3H), 1.55 (m, J=6.8 Hz, 1H), 1.27 (q, J=6.8 Hz, 2H), 0.79 (d, J=6.4 Hz, 6H); MS(ESI) m/e 470 (M+H)⁺.

### EXAMPLE 95

### 6-[(benzyloxy)methyl]-5-(4-{[4-(methylsulfonyl)benzyl]amino}phenyl)pyrimidine-2,4-diamine

The titled compound was prepared according to procedure in Example 2, substituting 4-methanesulfonyl-benzaldehyde for 4-chlorobenzaldehyde used in Example 2D. ¹H NMR (300 MHz, DMSO-d₆) δ 7.89 (d, J=8.5 Hz, 2H), 7.65 (d, J=8.5 Hz, 2H), 7.31-7.15 (m, 5H), 6.90 (d, J=8.5 Hz, 2H), 6.61 (d, J=8.8 Hz, 2H), 6.52 (t, J=6.3 Hz, 1H), 6.01 (s, 2H), 5.64 (s, 2H), 4.41 (d, J=6.1 Hz, 2H), 4.33 (s, 2H), 3.96 (s, 2H), 3.18 (s, 3H);MS (ESI) m/e 490 (M+H)⁺.

### EXAMPLE 96

### 5-(3-chloro-4-{[4-(methylsulfonyl)benzyl]amino}phenyl)-6-ethylpyrimidine-2,4-diamine

### EXAMPLE 96A

### 6-Ethyl-5-[4-(4-methanesulfonyl-benzylamino)-phenyl]-pyrimidine-2,4-diamine

The title compound was synthesized according to the procedure described in Example 1, substituting 4-methanesulfonyl-benzaldehyde for 4-chlorobenzaldehyde.

### EXAMPLE 96B

### 5-[3-Chloro-4-(4-methanesulfonyl-benzylamino)-phenyl]-6-ethyl-pyrimidine-2,4-diamine

To a mixture of Example 96A (40 mg, 0.1 mmol), acetic acid (20 μL), HClO₄ (500 μL) in a mixed solvent of dichloromethane (1 mL) and methanol (500 μL) was added N-chlorosuccinimide (13 mg, 0.1 mmol). The reaction mixture was stirred at room temperature for 48 hr. It was then partitioned between ethyl acetate and aqueous NaHCO₃ (20 mL, 1:1). The organic phase was washed with brine, dried (MgSO₄), filtered and concentrated under reduced pressure. The residue was purified by flash chromatography on silica gel column with ethyl acetate/methanol (10/1) to provide the titled compound (16 mg). ¹H NMR (300 MHz, DMSO-d₆) δ 7.90 (d, 2H, J=8.5 Hz), 7.65 (d, 2H, J=8.5 Hz), 7.05 (d, 1H, J=2.0 Hz), 6.84 (dd, 1H, J=2.0 Hz, J=8.1 Hz), 6.57(d, 1H, J=8.1 Hz), 6.35 (t, 1H, J=6.1 Hz), 5.80 (s, 2H), 5.42 (bs, 2H), 4.53 (d, 2H, J=6.1 Hz), 3.19 (s, 3H), 2.09 (q, 2H, J=7.5 Hz), 0.94 (t,3H, J=7.5 Hz); MS (ESI) positive ion 432 (M+H)⁺; negative ion 430 (M−H)⁻.

### EXAMPLE 97

### 6-[(benzyloxy)methyl]-5-(4-{[4-(methylsulfonyl)benzyl]oxy}phenyl)pyrimidine-2,4-diamine

The titled compound was prepared according to the procedure described in Example 56, substituting 1-bromomethyl-4-methanesulfonyl-benzene for 4-chlorobenzyl bromide used in Example 56 (64% yield). ¹H NMR (300 MHz, DMSO-D₆) δ 7.98 (d, J=8.5 Hz, 2H), 7.75 (d, J=8.5 Hz, 2H), 7.30-7.04 (m, 9H), 5.95 (s, 2H), 5.59 (s, 2H), 5.26 (s, 2H), 4.32 (s, 2H), 3.39 (s, 2H), 3.22 (s, 3H). MS (ESI) positive ion 491 (M+H)⁺; negative ion 489(M−H)⁻.

### EXAMPLE 98

### 6-{[methyl(phenyl)amino]methyl}-5-(4-{[4-(methylsulfonyl)benzyl]amino}phenyl)pyrimidine-2,4-diamine

### EXAMPLE 98A

### 6-bromomethyl-5-[4-(4-methanesulfonyl-benzylamino)-phenyl]-pyrimidine-2,4-diamine

6-[(Benzyloxy)methyl]-5-(4-{[4-(methylsulfonyl)benzyl]amino}phenyl)pyrimidine-2,4-diamine (620 mg, 1.27 mmol) was mixed with H₂O (5 mL) in a pressure vessel. HBr (33% in HOAC, 10 mL) was then added, the vessel was capped, and the reaction mixture was heated to 100° C. for 1 hour. The mixture was cooled in an ice bath followed by removal of solventin vacuo. Saturated aqueous NaHCO₃ (250 mL) was added followed by addition of EtO. The solution was stirred for 20 min. and sonicated. The resulting ppt. was filtered yielding 6-bromomethyl-5-[4-(4-methanesulfonyl-benzylamino)-phenyl]-pyrimidine-2,4-diamine (421 mg, 72%).

### EXAMPLE 98B

### 6-{[methyl(phenyl)amino]methyl}-5-(4-{[4-(methylsulfonyl)benzyl]amino}phenyl)pyrimidine-2,4-diamine

6-Bromomethyl-5-[4-(4-methanesulfonyl-benzylamino)-phenyl]-pyrimidine-2,4-diamine (20 mg, 0.043 mmol) was dissolved in THF (1 mL). Nmethylaniline (0.1 mL, 0.92 mmol) was added and the reaction mixture was stirred at ambient temperature overnight. The mixture was concentrated in vacuo, acidified with HCl (4M, 0.5 mL), taken up in MeOH (1.5 mL), and separated by RP-HPLC (5-100% CH₃CN in aq. NH₄OAc). The titled compound (9.2 mg, 44%) was obtained as an off white solid. ¹H NMR (300 MHz, DMSO-d6) δ 2.78 (s, 3H), 3.18 (s, 3 H), 3.98 (s, 2H), 4.42 (d, J=5.76 Hz, 2H), 5.38 (s, 2H), 5.72 (s, 2H), 6.40 (d, J=8.14 Hz, 2 H), 6.45-6.56 (m, 2 H), 6.65 (d, J=8.81 Hz, 2 H), 6.89 (d, J=8.48 Hz, 2 H), 7.00 (dd, J=8.81, 7.46 Hz, 2 H), 7.64 (d, J=8.48 Hz, 2 H), 7.89 (d, J=8.48 Hz, 2 H). MS (ESI) positive ion 489 (M+H)⁺; negative ion 487 (M−H)⁻.

### EXAMPLE 99

### 6-ethyl-5-(4-{methyl[4-(methylsulfonyl)benzyl]amino}phenyl)pyrimidine-2,4-diamine

The title compound was synthesized according to the procedure described in Example 43 and 44, substituting propionyl chloride for phenoxyacetyl chloride and 4-methanesulfonyl-benzaldehyde for 4-cyano-benzylaldehyde. ¹H NMR (300 MHz, DMSO-d₆) δ 7.90 (d, 2H, J=8.5 Hz), 7.52 (d, 2H, J=8.5 Hz), 6.97 (d, 2H, J=8.5 Hz), 6.78 (d, 2H, J=8.5 Hz), 5.74 (s, 2H), 5.28 (bs, 2H), 4.69(s, 2H), 3.20 (s, 3H), 3.07 (s, 3H), 2.12 (q, 2H, J=7.5 Hz), 0.95 (t, 3H, J=7.5 Hz); MS (ESI) positive ion 412 (M+H)⁺; negative ion 410 (M−H)⁻.

### EXAMPLE 100

### 6-(anilinomethyl)-5-(4-{[4-(methylsulfonyl)benzyl]amino}phenyl)pyrimidine-2,4-diamine

The titled compound was synthesized in the same manner as Example 98, substituting aniline for N-methylaniline. ¹H NMR (400 MHz, DMSO-d₆) δ 3.17 (s, 3H), 3.65 (d, J=4.91 Hz, 2H), 4.41 (d, J=6.14 Hz, 2 H), 5.45 (t, J=4.45 Hz, 1 H), 5.48 (s, 2H), 5.87 (s, 2 H), 6.38 (d, J=7.67 Hz, 2H), 6.45-6.54 (m, 2 H), 6.65 (d,J=8.59 Hz, 2 H), 6.93 (d, J=8.59 Hz, 2 H), 7.00 (t, J=7.83 Hz, 2 H), 7.65 (d, J=8.29 Hz, 2 H), 7.88 (d, J=8.29 Hz, 2 H). MS (ESI) positive ion 475 (M+H)⁺; 473 (M−H)⁻.

### EXAMPLE 101

### 6-[(benzyloxy)methyl]-5-(4-{[4-(ethylsulfonyl)benzyl]amino}phenyl)pyrimidine-2,4-diamine

The titled compound was synthesized according to the procedure described in Example 91, substituting 4-ethanesulfonyl-benzylamine for 3-fluoro-4-methanesulfonyl-benzylamine. ¹H NMR (300 MHz, DMSO-d₆) δ 7.85 (d, 2H, J=8.5 Hz), 7.66 (d, 2H, J=8.5 Hz), 7.30-7.15(m, 5H), 6.90 (d, 2H, J=8.5 Hz), 6.61 (d, 2H, J=8.5 Hz), 6.49 (t, 1H, J=6.1 Hz), 5.91 (s, 2H), 5.52 (bs, 2H), 4.41(d, 2H, J=6.1 Hz), 4.32 (s, 2H), 3.94 (s, 2H), 3.25 (q, 2H, J=7.5 Hz), 1.08 (t, 3H, J=7.5 Hz); MS (ESI) positive ion 504 (M+H)⁺; negative ion 502 (M−H)⁻.

### EXAMPLE 102

### 6-[(2-furylmethoxy)methyl]-5-(4-{[4-(methylsulfonyl)benzyl]amino}phenyl)pyrimidine-2,4-diamine

The title compound was prepared according to procedure in Example 89, substituting furan-2-yl-methanol for 3-methyl-2-butene-1-ol to give an off-white solid (14 mg, 17% yield). ¹H NMR (300 MHz, DMSO-d₆) δ 7.88-7.91 (d, J=8.48 Hz, 2H), 7.64-7.66 (d, J=8.48 Hz, 2H), 7.65 (s, 1H), 6.87-6.90 (d, 8.48 Hz, 2H), 6.58-6.61 (d, J=8.48 Hz, 2H), 6.47-6.51 (t, J=6.44 Hz, 5.76 Hz, 1H), 6.83-6.85 (m, 1H), 6.25-6.26 (d, J=3.05 Hz, 1H), 5.87 (s, 2H), 5.48 (s, 2H), 4.39-4.41 (d, J=6.10 Hz, 2H), 4.27 (s, 2H), 3.39 (s, 2H), 3.18 (s, 3H). MS (ESI) positive ion 480 (M+H)⁺; negative ion 478 (M−H)⁻.

### EXAMPLE 103

### 5-(4-{[4-(methylsulfonyl)benzyl]amino}phenyl)-6-neopentylpyrimidine-2,4-diamine

The titled compound was prepared according to the procedure described in Example 2, substituting 3,3-dimethyl-butyryl chloride for benzyloxyacetyl chloride, and 4-methanesulfonyl benzaldehyde for 4-chlorobenzaldehyde used in Example 2. ¹H NMR (300 MHz, DMSO-D₆) δ 7.88 (d, J=8.4 Hz, 2H), 7.64 (d, J=8.4 Hz, 2H), 6.82 (d, J=8.4 Hz, 2H), 6.63 (d, J=8.4 Hz, 2H), 6.40 (d, J=5.9 Hz, 1H), 5.64 (s, 2H), 5.22 (s, 2H), 4.40 (d, J=5.9 Hz, 2H), 3.18 (s, 3H), 2.15 (s, 2H), 0.77 (s, 9H); MS (ESI) positive ion 440 (M+H)⁺.

### EXAMPLE 104

### 1-[4-({[4-(2,4-diamino-6-isopropylpyrimidin-5-yl)phenyl]amino}methyl)phenyl]ethanol

### EXAMPLE 104A

### 1-(4-{[4-(2,4-Diamino-6-isopropyl-pyrimidin-5-yl)-phenylamino]-methyl}-phenyl)-ethanone

The titled compound was prepared according to the procedure described in Example 1, substituting 2-methyl-propionyl chloride for propionyl chloride, and 4-acetyl benzaldehyde for 4-chlorobenzaldehyde used in Example 2.

### EXAMPLE 104B

### 1-[4-({[4-(2,4-diamino-6-isopropylpyrimidin-5-yl)phenyl]amino}methyl)phenyl]ethanol

1-(4-{[4-(2,4-Diamino-6-isopropyl-pyrimidin-5-yl)-phenylamino]-methyl}-phenyl)-ethanone from Example 104A (15 mg, 0.040 mmol) was taken up in 1 mL of MeOH. NaBH₄ (4.5 mg, 0.12 mmol) was added. After 1 h at r.t., the reaction mixture was quenched with conc. HCl (1 drop) and purified on reverse phase prep. HPLC to give the titled compound (10 mg, 67%). ¹H NMR (300 MHz, DMSO-D₆) δ 7.33 (d, J=8.1 Hz, 2H), 7.30 (d, J=8.1 Hz, 2H), 6.83 (d, J=8.5 Hz, 2H), 6.64 (d, J=8.5 Hz, 2H), 6.26 (t, J=5.8 Hz, 1H), 5.68 (s, 2H), 5.21 (s, 2H), 5.09 (br s, 1H), 4.69 (q, J=6.2 Hz, 1H), 4.23 (d, J=5.7 Hz, 2H), 2.59 (p, J=6.8 Hz, 1H), 1.31 (d, J=6.4 Hz, 3H), 0.95 (d, J=6.8 Hz, 6H); MS (ESI) positive ion 378 (M+H)⁺.

### EXAMPLE 105

### 6-ethyl-5-{1-[4-(methylsulfonyl)benzyl]-1H-benzimidazol-5-yl}pyrimidine-2,4-diamine

The solution of Example 92 (10 mg) in formic acid (400 μL) was heated at 100° C. (oil bath) for 1 hr. It was then partitioned between ethyl acetate and aqueous NaHCO₃ (20 mL, 1:1). The organic phase was washed with brine, dried (MgSO₄), filtered and concentrated under reduced pressure. Purification by Reverse Phase Chromatography provided the titled compound (3 mg). ¹H NMR (300 MHz, DMSO-d₆) δ 8.48 (s, 1H), 7.93 (d, 2H, J=8.1 Hz), 7.63 (d, 2H, J=8.1 Hz), 7.60 (d, 1H, J=8.5 Hz), 7.44 (s, 1H), 7.03 (s, 1H), 5.80 (s, 2H), 5.65 (s, 2H), 5.38 (bs, 2H), 3.19 (s, 3H), 2.09 (q, 2H, J=7.5 Hz), 0.94 (t, 3H, J=7.5 Hz); MS (ESI) positive ion 423 (M+H)⁺; negative ion 421 (M−H)⁻.

### EXAMPLE 106

### 5-(4-{[3-chloro-4-(methylsulfonyl)benzyl]amino}phenyl)-6-ethylpyrimidine-2,4-diamine

### EXAMPLE 106A

### 3-chloro-4-methanesulfonyl-benzaldehyde

The titled compound was prepared as described in Example 91A, substituting 3-chloro-4-fluorobenzaldehyde for 3.4-difluorobenzaldehyde used in Example 91A.

### EXAMPLE 106B

### 5-(4-{[3-chloro-4-(methylsulfonyl)benzyl]amino}phenyl)-6-ethylpyrimidine-2,4-diamine

The titled compound was prepared according to procedure in Example 91B, substituting 5-(4-amino-phenyl)-6-ethyl-pyrimidine-2,4-diamine from Example 1C for 5-(4-amino-phenyl)-6-benzyloxymethyl-pyrimidine-2,4-diamine used in Example 91B and 3-chloro-4-methanesulfonyl-benzaldehyde for 3-fluoro-4-methanesulfonyl-benzaldehyde used in Example 91B. ¹H NMR (300 MHz, DMSO-d₆) δ 8.02 (d, J=8.1 Hz, 2H), 7.73 (d, J=1.4 Hz, 1H), 7.62 (dd, J=8.1, 1.7 Hz, 1H), 6.87 (d, J=8.5 Hz, 2H), 6.63 (d, J=8.5 Hz, 2H), 6.51 (t, J=6.3 Hz, 1H), 5.81 (s, 2H), 5.38 (s, 2H), 4.40 (d, J=6.1 Hz, 2H), 3.35 (s, 3H), 2.11 (q, J=7.5 Hz, 2H), 0.94 (t, J=7.6 Hz, 3H); MS (ESI) m/e 432, 434 (M+H)⁺.

### EXAMPLE 107

### 6-isopropyl-5-(4-{[4-(methylsulfonyl)benzyl]amino}phenyl)pyrimidine-2,4-diamine

The titled compound was prepared according to the procedure described in Example 2, substituting 2-methyl-propionyl chloride for benzyloxyacetyl chloride, and 4-methanesulfonyl benzaldehyde for 4-chlorobenzaldehyde used in Example 2. ¹H NMR (300 MHz, DMSO-d₆) δ 7.90 (d, J=8.2 Hz, 2H), 7.66 (d, J=8.2 Hz, 2H), 6.85 (d, J=8.5 Hz, 2H), 6.63 (d, J=8.5 Hz, 2H), 6.5 (t, J=6.1 Hz, 1H), 5.70 (s, 2H), 5.23 (s, 2H), 4.40 (d, J=6.1 Hz, 2H), 3.20 (s, 3H), 2.58 (m, 1H), 0.95 (d, J=6.7 Hz, 6H); MS (ESI) m/e 412 (M+H)⁺.

### EXAMPLE 108

### 1-(4-{[(4-{2,4-diamino-6-[(benzyloxy)methyl]pyrimidin-5-yl}phenyl)amino]methyl}phenyl)propan-1-one

The titled compound was synthesized according to the procedure described in Example 91, substituting 1-(4-aminomethyl-phenyl)-propan-1-one for 3-fluoro-4-methanesulfonyl benzylamine. ¹H NMR (300 MHz, DMSO-d₆) δ 7.93 (d, 2H, J=8.5 Hz), 7.53 (d, 2H, J=8.5 Hz), 7.30-7.15(m, 5H), 6.90 (d, 2H, J=8.5 Hz), 6.61 (d, 2H, J=8.5 Hz), 6.45 (t, 1H, J=6.1 Hz), 5.87 (s, 2H), 5.48 (bs, 2H), 4.37(d, 2H, J=6.1 Hz), 4.31 (s, 2H), 3.93 (s, 2H), 3.01 (q, 2H, J=7.5 Hz), 1.07 (t, 3H, J=7.5 Hz); MS (ESI) positive ion 468 (M+H)⁺; negative ion 466 (M−H)⁻.

### EXAMPLE 109

### 6-{[(2,3-dimethoxyphenyl)amino]methyl}-5-(4-{[4-(methylsulfonyl)benzyl]amino}phenyl)pyrimidine-2,4-diamine

In a microwave vial, 6-bromomethyl-5-[4-(4-methanesulfonyl-benzylamino)-phenyl}-pyrimidine-2,4-diamine from 98 (26 mg, 0.06 mmol) was dissolved in DMA (0.7 mL).Then 2,3-dimethoxyaniline (73 mg, 0.48 mmol) dissolved in DMA (1.8 mL) was added followed by the addition of DIEA (30 μL, 0.18 mmol) in DMA (0.7 mL). The mixture was heated in the microwave to 100° C. for 900 seconds. The crude mixture was purified using reverse phase HPLC. 1H NMR (500 MHz, DMSO-D₆/D₂O) δ ppm 3.17-3.21 (m, 3 H), 3.64-3.67 (m, 3 H), 3.67-3.71 (m, 2 H), 3.72-3.76 (m, 3 H), 4.42 (d, 2 H), 5.38-5.57 (m, 2 H), 5.89 (d, 1 H), 6.30 (d, 1 H), 6.49-6.58 (m, 1 H), 6.67 (d, 2 H), 6.76 (t, 1 H), 6.94 (d, 2H), 7.65 (d, 2 H), 7.89 (d, 2 H). MS (ESI) positive ion 535 (M+H)⁺; negative ion 533 (M−H)⁻.

### EXAMPLE 110

### 6-ethyl-5-(4-{[3-methyl-4-(methylsulfonyl)benzyl]amino}phenyl)pyrimidine-2,4-diamine

### EXAMPLE 110A

### 3-Methyl-4-methanesulfonyl-benzaldehyde

The titled compound was prepared as described in Example 91A, substituting 3-methyl-4-fluorobenzaldehyde for 3,4-difluorobenzaldehyde used in Example 91A.

### EXAMPLE 110B

### 6-Ethyl-5-(4-{[3-methyl-4-(methylsulfonyl)benzyl]amino}phenyl)pyrimidine-2,4-diamine

The titled compound was prepared according to procedure in Example 91B, substituting 5-(4-amino-phenyl)-6-ethyl-pyrimidine-2,4-diamine from Example 1C for 5-(4-amino-phenyl)-6-benzyloxymethyl-pyrimidine-2,4-diamine used in Example 91B and 3-methyl-4-methanesulfonyl-benzaldehyde for 3-fluoro-4-methanesulfonyl-benzaldehyde used in Example 91B. ¹H NMR (300 MHz, DMSO-d₆) δ 7.87 (d, J=8.5 Hz, 1H), 7.47 (br s, 1H), 7.45 (br s, 1H), 6.86 (d, J=8.5 Hz, 2H), 6.62 (d, J=8.8 Hz, 2H), 6.42 (t, J=5.93 Hz, 1H), 5.72 (s, 2H), 5.26 (s, 2H), 4.34 (d, J=6.1 Hz, 2H), 3.19 (s, 3H), 2.63 (s, 3H), 2.11 (q, J=7.6 Hz, 2H), 0.94 (t, J=7.6 Hz, 3H); MS (ESI) m/e 428 (M+H)⁺.

### EXAMPLE 111

### 5-(4-{[3,5-difluoro-4-(methylsulfonyl)benzyl]amino}phenyl)-6-ethylpyrimidine-2,4-diamine

### EXAMPLE 111A

### 3,5-difluoro-4-methanesulfonyl-benzaldehyde

The titled compound was prepared as described in Example 91A, substituting 3,4,5-trifluorobenzaldehyde for 3,4-difluorobenzaldehyde used in Example 91A.

### EXAMPLE 111B

### 5-(4-{[3,5-difluoro-4-(methylsulfonyl)benzyl]amino}phenyl)-6-ethylpyrimidine-2,4-diamine

The titled compound was prepared according to procedure in Example 91B, substituting 5-(4-amino-phenyl)-6-ethyl-pyrimidine-2,4-diamine from Example 1C for 5-(4-amino-phenyl)-6-benzyloxymethyl-pyrimidine-2,4-diamine used in Example 91B and 3,5-difluoro-4-methanesulfonyl-benzaldehyde for 3-fluoro-4-methanesulfonyl-benzaldehyde used in Example 91B. ¹H NMR (300 MHz, DMSO-d₆) δ 7.33 (d, J=10.2 Hz, 2H), 6.88 (d, J=8.5 Hz, 2H), 6.61 (d, J=8.5 Hz, 2H), 6.50 (t, J=6.3 Hz, 1H), 5.72 (s, 2H), 5.27 (s, 2H), 4.39 (d, J=6.1 Hz, 2H), 3.41 (s, 3H), 2.11(q, J=7.6 Hz, 2H), 0.94 (t, J=7.6 Hz, 3H);MS (ESI) m/e 434 (M+H)⁺.

### EXAMPLE 112

### 6-[(benzyloxy)methyl]-5-[4-({4-[(trifluoromethyl)sulfonyl]benzyl}amino)phenyl]pyrimidine-2,4-diamine

The titled compound was synthesized according to the procedure described in Example 91, substituting 4-trifluoromethanesulfonyl-benzylamine for 3-fluoro-4-methanesulfonyl-benzylamine. ¹H NMR (300 MHz, DMSO-d₆) δ 8.11 (d, 2H, J=8.5 Hz), 7.85 (d, 2H, J=8.5 Hz), 7.30-7.15(m, 5H), 6.92 (d, 2H, J=8.5 Hz), 6.61 (d, 2H, J=8.5 Hz), 6.57 (t, 1H, J=6.1 Hz), 5.89 (s, 2H), 5.51 (bs, 2H), 4.51(d, 2H, J=6.1 Hz), 4.31 (s, 2H), 3.94 (s, 2H); MS ( ESI) positive ion 544 (M+H)⁺; negative ion 542 (M−H)⁻.

### EXAMPLE 113

### 6-ethyl-5-{1-[4-(methylsulfonyl)benzyl]-1H-indol-5-yl}pyrimidine-2,4-diamine

### EXAMPLE 113A

### 6-Ethyl-5-(1H-indol-5-yl)-pyrimidine-2,4-diamine

The reaction mixture of 6-ethyl-5-iodo-pyrimidine-2,4-diamine (60 mg, 0.23 mmol), 5-indolylboronic acid (41 mg, 0.25 mmol), tetrakis,(triphenyl-phosphine)-palladium (13 mg, 5%), Na₂CO₃ and toluene (2 ml) in a sealed tube was heated at 100° C. for 2 hours, and then the solvent was removed by evaporator and the residue was purified by reverse phase HPLC (070% CH₃CN in aq. NH₄OAc) providing the title compound (40 mg, 70.1%). ¹H NMR (300 MHz, DMSO-d₆) δ 11.13 (s, 1H), 7.45 (d, J=9.0 Hz, 1H), 7.36 (t, J=3.0 Hz, 1H), 7.33 (d, J=3.0 Hz, 1H), 6.86 (dd, J₁=9.0 Hz, J₂=3.0 Hz, 1H), 5.77 (s, 2H), 5.29 (s, 2H), 2.13 (q, J=7.5 Hz, 2H), 0.95 (t, J=7.5 Hz, 3H). MS (ESI) positive ion 254 (M+H)⁺; negative ion 252 (M−H)⁻.

### EXAMPLE 113B

### 6-Ethyl-5-{1-[4-(methylsulfonyl)benzyl]-1H-indol-5-yl}pyrimidine-2,4-diamine

To a mixture of 6-ethyl-5-(1H-indol-5-yl)-pyrimidine-2,4-diamine (28 mg, 0.1 mmol) from Example 113A and 1-bromomethyl-4-methanesulfonyl-benzene (30 mg, 0.12 mmol) in DMSO (1.5 ml) was added KOH (13 mg, 0.12 mmol). The mixture was stirred at room temperature for 4 hours and checked by LC/Ms. After reaction finished, the crude product was purified by reverse phase HPLC (0-70% CH₃CN in aq. NH₄OAc) providing the titled compound (20 mg, 43% yield). ¹H NMR (300 MHz, DMSO-D₆) δ 7.90 (d, J=9.0 Hz, 2H), 7.58 (d, J=3.0 Hz, 1H), 7.53 (d, J=9.0 Hz, 3H), 7.36 (s, 1H), 6.90 (dd, J₁=9.0 Hz, J₂=3.0 Hz, 1H), 6.54 (d, J=3.0 Hz, 1H), 5.79 (s, 2H), 5.57 (s, 2H), 5.37 (s, 2H), 3.18 (s, 3H), 2.11 (q, J=7.5 Hz, 2H), 0.95 (t, J=7.5 Hz, 3H). MS (ESI) positive ion 422 (M+H)⁺; negative ion 420 (M−H)⁻.

### EXAMPLE 114

### 6-ethyl-5-(4-{[4-(methylsulfonyl)benzyl]amino}phenyl)pyrimidine-2,4-diamine

The titled compound was synthesized according to the procedure described in Example 1D, substituting 4-methanesulfonyl-benzaldehyde for 4-chlorobenzaldehyde. ¹H NMR (300 MHz, DMSO-d₆) δ 7.90 (d, J=8.5 Hz, 2H), 7.65 (d, J=8.5 Hz, 2H), 6.88 (d, J=8.5 Hz, 2H), 6.64 (d, J=8.5 Hz, 2H), 6.54 (t, J=5.9 Hz, 1H), 6.21 (s, 2H), 5.86 (s, 2H), 4.40 (d, J=6.1 Hz, 2H), 3.20 (s, 3H), 2.14 (q, J=7.6 Hz, 2H), 0.97 (t, J=7.6 Hz, 3H); MS (ESI) m/e 398 (M+H)⁺.

### EXAMPLE 115

### methyl 4-{[(4-{2,4-diamino-6-[(benzyloxy)methyl]pyrimidin-5-yl}phenyl)amino]methyl}benzoate

The titled compound was synthesized in the same manner as Example 91, substituting methyl 4-formylbenzoate for 3-fluoro-4-(methylsulfonyl)benzyaldehyde. ¹H NMR (300 MHz, DMSO-d₆) δ 3.83 (s, 3 H), 3.94 (s, 2 H), 4.31 (s, 2 H), 4.37 (d, J=5.76 Hz, 2 H), 5.58 (s, 2 H), 5.94 (s, 2 H), 6.46 (t, J=6.27 Hz, 1 H), 6.60 (d, J=8.48 Hz, 2 H), 6.89 (d, J=8.82 Hz, 2 H), 7.14-7.19 (m, 2 H), 7.20-7.33 (m, 3 H), 7.53 (d,J=8.48 Hz, 2 H), 7.93 (d, J=8.48 Hz, 2 H). MS (ESI) positive ion 470 (M+H)⁺; negative ion 468 (M−H)⁻.

### EXAMPLE 116

### 6-[(benzylamino)methyl]-5-(4-{[4-(methylsulfonyl)benzyl]amino}phenyl)pyrimidine-2,4-diamine

The titled compound was synthesized in the same manner as Example 98, substituting benzylamine for N-methylaniline. ¹H NMR (300 MHz, DMSO-d₆) δ 3.17 (d, J=1.70 Hz, 2 H), 3.19 (s, 3 H), 3.56 (s, 2 H), 4.39 (d, J=5.76 Hz, 2 H), 5.42 (s, 2 H), 5.84 (s, 2 ), 6.49 (t, J=5.76 Hz, 1 H), 6.59 (d, J=8.48 Hz, 2 H), 6.86 (d, J=8.48 Hz, 2 H), 7.14-7.31 (m, 5 H), 7.64 (d, J=8.14 Hz, 2 H), 7.89 (d, J=8.48 Hz, 2 H). MS (ESI) positive ion 489 (M+H)⁺; negative ion 487 (M−H)⁻.

### EXAMPLE 117

### 1-(4-{[4-(2,4-diamino-6-ethylpyrimidin-5-yl)benzyl]amino}phenyl)ethanone

### EXAMPLE 117A

### 4-(2,4-Diamino-6-ethyl-pyrimidin-5-yl)-benzonitrile

The titled compound was prepared according to the procedure described in Example 1, substituting 4-cyanomethyl-benzonitrile for 4-nitrophenylacetonitrile used in Example 1 (61% yield). ¹H NMR (300 MHz, DMSO-d₆) δ 7.87 (d, J=8.5 Hz, 2H), 7.39 (d, J=8.5 Hz, 2H), 5.93 (s, 2H), 5.63 (s, 2H), 2.09 (q, J=9.0 Hz, 2H), 0.95 (t, J=9.0 Hz, 3H). MS (ESI) positive ion 240(M+H)⁺; negative ion 238(M−H)⁻.

### EXAMPLE 117B

### 4-(2,4-Diamino-6-ethyl-pyrimidin-5-yl)-benzaldehyde

To 4-(2,4-diamino-6-ethyl-pyrimidine-5-yl)-benzonitrile (479 mg, 2 mmol) from Example 117A in 10 mL of formic acid (75%,v/v) was added nickl aluminide (20 mesh, 558 mg, 2 mmol). The mixture was refluxed under nitrogen for 3 h and filtered. The solid was washed with ethanol twice. The combined filterate was concentrated under reduced pressure to dryness. The residue was purified by chromatography on column of silica gel providing the titled compound (450 mg, 93%). ¹H NMR (300 MHz, DMSO-d₆) δ 9.78 (s, 1H), 7.85 (d, J=8.5 Hz, 2H), 7.36 (d, J=8.5 Hz, 2H), 5.91 (s, 2H), 5.62 (s, 2H), 2.09 (q, J=9.0 Hz, 2H), 0.95 (t, J=9.0 Hz, 3H). MS (ESI) positive ion 243 (M+H)⁺; negative ion 241(M−H)⁻.

### EXAMPLE 117C

### 1-(4-{[4-(2,4-Diamino-6-ethylpyrimidin-5-yl)benzyl]amino}phenyl)ethanone

To 1-(4-amino-phenyl)-ethanone (30 mg, 0.22 mmol) in methanol (2 ml) and a buffer solution of acetic acid and sodium acetate (1 mL, pH 4-5) 4-(2,4-diamino-6-ethyl-pyrinidine-5-yl)-benzaldehyde (48.4 mg, 0.2 mmol) from Example 117B was added and the mixture stirred for 10 minutes, and then NaBH₃CN (138 mg, 0.22 mmol). The reaction mixture was stirred for 2 hours at 25° C. and checked with LC/Ms. After the reaction finished, solvent was removed by evaporator and the residue was purified by reverse phase HPLC (070% CH₃CN in aq. NH₄Oac) to provide the titled compound (50.2 mg, 69.5%). ¹H NMR (300 MHz, DMSO-d₆) δ 7.72 (d, J=8.5 Hz, 2H), 7.40 (d, J=8.5 Hz, 2H), 7.18 (t, J=7.5 Hz, 1H), 7.16 (d, J=8.5 Hz, 2H), 6.66 (d, J=8.5 Hz, 2H), 5.87 (s, 2H), 5.40 (s, 2H), 4.40 (d, J=6.0 Hz, 2H), 2.39 (s, 3H), 2.10 (q, J=7.5 Hz, 2H), 0.95 (t, J=7.5 Hz, 3H). MS(ESI) positive ion 362 (M+H)⁺; negative ion 360 (M−H)⁻.

### EXAMPLE 118

### 6-(methoxymethyl)-5-(4-{[4-(methylsulfonyl)benzyl]amino}phenyl)pyrimidine-2,4-diamine

The titled compound was prepared according to the procedure described in Example 2, substituting methoxyacetyl chloride for benzyloxyacetyl chloride, and 4-methanesulfonyl benzaldehyde for 4-chlorobenzaldehyde used in Example 2 (67% yield). ¹H NMR (300 MHz, DMSO-d₆) δ 7.90 (d, J=8.5 Hz, 2H), 7.65 (d, J=8.5 Hz, 2H), 6.89 (d, J=8.5 Hz, 2H), 6.62 (d, J=8.5 Hz, 2H), 6.49 (t, J=6.0 Hz, 1H), 5.85 (s, 2H), 5.46 (s, 2H), 4.40 (d, J=6.0 Hz, 2H), 3.82 (s, 2H), 3.19 (s, 3H), 3.09 (s, 3H). MS (ESI) positive ion 414 (M+H)⁺; negative ion 412(M−H)⁻.

### EXAMPLE 119

### 6-ethyl-5-(4-{[(4-nitrophenyl)amino]methyl}phenyl)pyrimidine-2,4-diamine

The titled compound was prepared according to the procedure described in Example 117, substituting 4-nitro-phenylamine for 1-(4-amino-phenyl)-ethanone used in Example 117C (70% yield). ¹H NMR (300 MHz, DMSO-d₆) δ 8.01 (d, J=9.0 Hz, 2H), 7.83 (t, J=6.0 Hz, 1H), 7.41 (d, J=9.0 Hz, 2H), 7.18 (d, J=8.5 Hz, 2H), 6.73 (d, J=8.5 Hz, 2H), 5.86 (s, 2H), 5.37 (s, 2H), 4.46 (d, J=6.0 Hz, 2H), 2.10 (q, J=7.5 Hz, 2H), 0.95 (t, J=7.5 Hz, 3H). MS(ESI) positive ion 365 (M+H)⁺; negative ion 363 (M−H)⁻.

### EXAMPLE 120

### 6-[(benzyloxy)methyl]-5-(4-{[4-(1,3-oxazol-2-yl)benzyl]amino}phenyl)pyrimidine-2,4-diamine

### EXAMPLE 120A

### 4-Oxazol-2-yl-benzaldehyde

To 4-chlorocarbonyl-benzoic acid methyl ester (792 mg, 4 mmol) in CH₂Cl₂ (10 mL) was added 2,2-dimethoxy-ethylamine (420 mg, 4 mmol) and Et₃N (505 mg, 5 mmol). This reaction mixture was allowed to stir overnight and usual work-up followed by column chromatography purification produced N-(2,2-Dimethoxy-ethyl)-terephthalamic acid methyl ester (580 mg, 54%). To this material was added Eaton's reagent (7.7% P₂O₅ in MeSO₃H, w/w, 4.5 mL) and the mixture was heated to 140° C. for 2 h. The mixture was poured into NaOH (10 M, 5 mL) in ice chips (50 g) and extracted with EtOAC to give a mixture of 4-Oxazol-2-yl-benzoic acid methyl ester and methyl methanesulfonate (700 mg, 1:4). This crude material was dissolved in THF (8 mL) and treated with DIBAL-H (1 M, 2 mL, 2 mmol) at −78° C. The reaction mixture was quenched after 30 min and purified to give (4-Oxazol-2-yl-phenyl)-methanol (90 mg) (4 Oxazol-2-yl-benzoic acid methyl ester (100 mg) was recovered). To (4Oxazol-2-yl-phenyl)-methanol in CH₂Cl₂ (6 mL) was added Dess-Martin reagent (270 mg, 0.6 mmol) and the mixture was allowed to stir for 2 h and purified by column chromatography to give the titled 4-oxazol-2-yl-benzaldehyde (40 mg).

### EXAMPLE 120B

### 6-[(Benzyloxy)methyl]-5-(4-{[4-(1,3-oxazol-2-yl)benzyl]amino}phenyl)pyrimidine-2,4-diamine

The aldehyde from Example 120A (17.3 mg, 0.1 mmol) and 5-(4-amino-phenyl)-6-benzyloxymethyl-pyrimidine-2,4-diamine (32 mg, 0.1 mmol) were dissolved in MeOH buffer (1 M, NaOAc/HOAC, pH4, 1.5 mL) followed by addition of NaBH₃CN (6.2 mg, 0.1 mmol). After 20 min, the reaction mixture was quenched with conc. HCl (aq, 3 drops) and purified by HPLC to give the titled compound (20 mg, 41%). ¹H NMR (400 MHz, DMSO-d₆) δ 8.18 (1H), 7.95 (d, J=8.4 Hz, 2H), 7.55 (d, J=8.0 Hz, 2H), 7.35 (s, 1H), 7.27-7.15 (m, 5H), 6.90 (d, J=8.8 Hz, 2H), 6.63 (d, J=8.4 Hz, 2H), 6.41 (t, J=6.0 Hz, 1H), 5.85 (s, 2H), 5.47 (s, 2H), 4.36 (d, J=5.6 Hz, 2H), 4.31 (s, 2H), and 3.94 (s, 2H). MS (ESI) m/e positive ion 479 (M+H)⁺.

### EXAMPLE 121

### 6-hexyl-5-(4-{[4-(methylsulfonyl)benzyl]amino}phenyl)pyrimidine-2,4-diamine

The titled compound was prepared according to the procedure described in Example 2, substituting n-heptanoyl chloride for benzyloxyacetyl chloride, and 4-methanesulfonyl benzaldehyde for 4-chlorobenzaldehyde used in Example 2. ¹H NMR (300 MHz, DMSO-d₆) δ 7.89 (d, J=8.2 Hz, 2H), 7.65 (d, J=8.2 Hz, 2H), 6.84 (d, J=8.5 Hz, 2H), 6.62 (d, J=8.5 Hz, 2H), 6.46 (t, J=6.1 Hz, 1H), 5.74 (s, 2H), 5.28 (s, 2H), 4.40 (d, J=5.8 Hz, 2H), 3.19 (s, 3H), 2.09 (t, J=7.9 Hz, 2H), 1.44-1.35 (m, 2H), 1.20-1.11 (m, 2H), 1.11-1.03 (m, 4H), 0.78 (t, J=7.2 Hz, 3H); MS (ESI) m/e 454 (M+H)⁺.

### EXAMPLE 122

### 6-But-3-enyl-5-(4-{[4-(methylsulfonyl)benzyl]amino}phenyl)pyrimidine-2,4-diamine

The titled compound was prepared according to the procedure described in Example 2, substituting pent-4-enoyl chloride for benzyloxyacetyl chloride, and 4-methanesulfonyl benzaldehyde for 4-chlorobenzaldehyde used in Example 2. ¹H NMR (300 MHz, DMSO-d₆) δ 7.89 (d, J=8.5 Hz, 2H), 7.65 (d, J=8.5 Hz, 2H), 6.86 (d, J=8.5 Hz, 2H), 6.63 (d, J=8.5 Hz, 2H), 6.47 (t, J=6.1 Hz, 2H), 5.73 (s, 2H), 5.28 (s, 2H), 5.67 (m, 1H), 4.86 (d, J=6.1 Hz, 1H), 4.82 (s, 1H), 4.40 (d, J=5.8 Hz, 2H), 3.19 (s, 3H), 2.18 (m, 2H); MS (ESI) m/e 424 (M+H)⁺.

### EXAMPLE 123

### 6-ethyl-N˜4˜-methyl-5-(4-{[4-(methylsulfonyl)benzyl]amino}phenyl)pyrimidine-2,4-diamine

### EXAMPLE 123A

### Propionic acid 1-(2-bromo-ethoxy)-2-(4-nitro-phenyl)-3-oxo-pent-1-enyl ester

To 4-nitrophenylacetic acid (10.8 g, 60 mmol) in benzene (25 mL) was added SOCl₂(25 mL). The mixture was heated at 100° C. for 30 min and the volatiles were removed under vacuum. CH₂Cl₂ (60 mL) and 2-bromoethanol (7.5 g, 60 mmol) were added at 0° C. followed by Et₃N (7.3 g, 73 mmol). Et₃N.HCl was filtered off after 30 min and Et₃N (22 g, 220 mmol) was added to the filtrate at 0° C. Propionyl chloride (20 g, 210 mmol) was added dropwise and the mixture was stirred for 2 h, quenched, and purified by column chromatography to give propionic acid 1-(2-bromo-ethoxy)-2-(4-nitro-phenyl)-3-oxo-pent-1-enyl ester (10.2 g, 43% over 3 steps).

### EXAMPLE 123B

### 2-[2-amino-6-ethyl-5-(4-nitro-phenyl)-pyrimidin-4-yloxy]-ethanol

To the ester from Example 123A (8 g, 20 mmol) in DMF (120 mL) was added NaH (50%, 2.4 g, 50 mmol). After 3 days, the reaction mixture was quenched with NH₄Claq at −20° C. and extracted with EtOAC (150 mL) and the organic extract was washed with water (3×50 mL). Column chromatography purification provided 1-[1,3]dioxolan-2-ylidene-1-(4-nitro-phenyl)-butan-2-one (1.8 g, 34%) along with propionic acid 1-[[1,3]dioxolan-2-ylidene-(4-nitro-phenyl)-methyl]-propenyl ester (0.8 g, 13%). These two compounds were combined and dissolved in EtOH (25 mL) and guanidine hydrochloride (3.4 g, 34 mmol) and NaOEt (2.5 M, 15 mL, 37.5 mmol) were added. The reaction mixture was heated at 85° C. for 1 h. EtOH was removed and water was added. The precipitates were collected and washed with water to give the titled 2-[2-amino-6-ethyl-5-(4-nitro-phenyl)-pyrimidin-4-yloxy]-ethanol (2 g, 70%).

### EXAMPLE 123C

### 4-Cloro-6-ethyl-5-(4-nitro-phenyl)-pyrimidin-2-ylamine

The alcohol from Example 123B (108 mg, 0.36 mmol) was added to a mixture of TMSBr (0.25 mL) and 1,4-dioxane (0.25 mL). The mixture was heated at 170° C. under microwave irradiation for 20 min. The precipitates were collected and washed with 1,4-dioxane to give 2-amino-6-ethyl-5-(4-nitro-phenyl)-pyrimidin-4-ol (98 mg, ˜100%). To this material was added POCl₃ (1 mL) and the mixture was heated at 150° C. under microwave irradiation for 20 min. POCl₃ was removed and ice chips were added. NaHCO₃aq was added to adjust pH to 7. Precipitates were collected to give the titled 4-cloro-6-ethyl-5-(4-nitro-phenyl)-pyrimidin-2-ylamine (80 mg, 85% over 2 steps).

### EXAMPLE 123D

### 6-Ethyl-N4-methyl-5-(4-nitro-phenyl)-pyrimidine-2,4-diamine

To the chloride from Example 123C (40 mg, 0.14 mmol) in 1,4-dioxane (0.5 mL) was added MeNH₂.HCl (68 mg, 1 mmol) and Et₃N (101 mg, 1 mmol). The mixture was heated at 170° C. under microwave irradiation for 25 min. Water was added and the precipitates were collected to give 6-ethyl-N4-methyl-5-(4-nitro-phenyl)-pyrimidine-2,4-diamine (25 mg, 65%).

### EXAMPLE 123E

### 6-ethyl-N-4-methyl-5-(4-{[4-(methylsulfonyl)benzyl]amino}phenyl)pyrimidine-2,4-diamine

The nitro compound from Example 123D (25 mg) was dissolved in MeOH buffer (1 M, NaOAc/HOAc, pH4, 1.5 mL) and Pd/C (10%, 10 mg) was added. H₂ balloon was applied. 4-Methanesulfonyl-benzaldehyde (18 mg, 0.1 mmol) and NaBH₃CN (6.2 mg, 0.1 mmol) were added after 1 h. Pd/C was filtered off after 1 h and the filtrate was purified by HPLC to give the titled 6-ethyl-5-[4-(4-methanesulfonyl-benzylamino)-phenyl]-N-4-methyl-pyrimidine-2,4-diamine (20 mg, 35% over 3 steps). ¹H NMR (400 MHz, DMSO-d₆) δ 7.90 (d, J=8.0 Hz, 2H), 7.66 (d, J=8.4 Hz, 2H), 6.84 (d, J=8.4 Hz, 2H), 6.63 (d, J=8.4 Hz, 2H), 6.48 (t, J=6.0 Hz, 1H), 5.96 (s, 2H), 5.28 (s, 2H), 4.39 (d, J=6.0 Hz, 2H), 2.68 (d, J=5.2 Hz, 3H), 2.07 (quartet, J=6.8 Hz, 2H), and 0.94 (d, J=7.2 Hz, 3H). MS (ESI) m/e positive ion 412 (M+H)⁺; negative ion 410 (M−H)⁺.

### EXAMPLE 124

### 6-ethyl-5-[4-({[4-(methylsulfonyl)phenyl]amino}methyl)phenyl]pyrimidine-2,4-diamine

The titled compound was prepared according to the procedure described in Example 117, substituting 4-methanesulfonyl-phenylamine for 1-(4-amino-phenyl)-ethanone used in Example 117C (72% yield). ¹H NMR (300 MHz, DMSO-d₆) δ 7.56 (d, J=9.0 Hz, 2H), 7.41 (d, J=9.0 Hz, 2H), 7.26 (t, J=6.0 Hz, 1H), 7.16 (d, J=8.5 Hz, 2H), 6.73 (d, J=8.5 Hz, 2H), 5.87 (s, 2H), 5.38 (s, 2H), 4.40 (d, J=6.0 Hz, 2H), 2.11 (q, J=7.5 Hz, 2H), 0.96 (t, J=7.5 Hz, 3H). MS (ESI) positive ion 398 (M+H)⁺; negative ion 396 (M−H)⁻.

### EXAMPLE 125

### 6-(2,3-dihydro-1H-indol-1-ylmethyl)-5-(4-{[4-(methylsulfonyl)benzyl]amino}phenyl)pyrimidine-2,4-diamine

The titled compound was synthesized in the same manner as Example 98, substituting indoline for N-methylaniline. ¹H NMR (500 MHz, DMSO-d₆) δ 2.72 (t, J=8.42 Hz, 2 H), 3.14 (t, J=8.42 Hz, 2 H), 3.18 (s, 3 H), 3.69 (s, 2 H), 4.41 (d, J=5.93 Hz, 2 H), 5.41 (s, 2 H), 5.80 (s, 2 H), 6.13 (d, J=7.80 Hz, 1 H), 6.41-6.52 (m, 2 H), 6.62 (d, J=8.42 Hz, 2 H), 6.80 (t, J=7.64 Hz, 1 H), 6.87-6.94 (m, J=8.27, 8.27 Hz, 3 H), 7.63 (d, J=8.11 Hz, 2 H), 7.88 (d, J=8.42 Hz, 2 H). MS (ESI) positive ion 501 (M+H)⁺; negative ion 499 (M−H)⁻.

### EXAMPLE 126

### 6-(3,4-dihydroisoguinolin-2(1H)-ylmethyl)-5-(4-{[4-(methylsulfonyl)benzyl]amino}phenyl)pyrimidine-2,4-diamine

The title compound was synthesized in the same manner as Example 98, substituting 1,2,3,4-tetrahydroquinoline for N-methylaniline. ¹H NMR (300 MHz, DMSO-d₆) δ 2.56 (t, J=5.93 Hz, 2 H), 2.63-2.75 (m, 2 H), 3.12 (s, 2 H), 3.18 (s, 3 H), 3.43 (s, 2 H), 4.40 (d, J=6.10 Hz, 2 H), 5.48 (s, 2 H), 5.91 (s, 2 H), 6.47 (t, J=5.93 Hz, 1 H), 6.61 (d, J=8.48 Hz, 2 H), 6.94 (d, J=8.48 Hz, 2 H), 6.94 (m, 1 H), 7.01-7.16 (m, 3 H), 7.64 (d, J=8.14 Hz, 2 H), 7.89 (d, J=8.14 Hz, 2 H). MS (ESI) positive ion 515 (M+H)⁺; negative ion 513 (MH)⁻.

### EXAMPLE 127

### 5-(4-{[4-(methylsulfonyl)benzyl]amino}phenyl)-6-{[(thien-2-ylmethyl)amino]methyl}pyrimidine-2,4-diamine

The titled compound was synthesized according to the procedure described in Example 98, substituting thiophen-2-yl-methylamine for aniline. ¹H NMR (300 MHz, DMSO-d₆) δ 7.90 (d, 2H, J=8.5 Hz), 7.65 (d, 2H, J=8.5 Hz), 7.32 (dd, 1H, J=1.4 Hz, J=5.1 Hz), 6.90-6.83(m, 4H), 6.60 (d, 2H, J=8.5 Hz), 6.48 (t, 1H, J=5.8 Hz), 5.83 (s, 2H), 5.41 (bs, 2H), 4.39(d, 2H, J=5.8 Hz), 3.76 (s, 2H), 3.21 (s, 2H), 3.19 (s, 3H); MS (ESI) positive ion 495 (M+H)⁺; negative ion 493 (M−H)⁻.

### EXAMPLE 128

### 6-bicyclo[2.2.1]hept-2-yl-5-(4-{[4-(methylsulfonyl)benzyl]amino}phenyl)pyrimidine-2,4-diamine

The titled compound was synthesized according to the procedure described in Example 1A-1D, substituting bicyclo[2.2.1]heptane-2-carbonyl chloride for propionyl chloride used in Example 1A, and 4 methanesulfonyl benzaldehyde for 4-chlorobenzaldehyde used in Example 1D. ¹H NMR (A 2:1 mixture of both endo and exo isomers, 300 MHz, DMSO-d₆) δ 7.89 (d, J=7.9 Hz, 2H), 7.65 (d, J=7.8 Hz, 2H), 6.91 (br m, 2H), 6.66 (br m, 2H), 4.42 (br m, 2H), 3.19 (s, 3H), 2.28 (m, 1H), 1.65-0.91 (m, 10H); MS (ESI) m/e 464 (M+H)⁺.

### EXAMPLE 129

### N-4-benzyl-5-(4-{[4-(methylsulfonyl)benzyl]amino}phenyl)pyrimidine-2,4,6-triamine

### EXAMPLE 129A

### 5-Bromo-6-chloro-pyrimidine-2,4-diamine

Bromine (514 μL, 10 mmol) was added dropwise to a solution of 6-chloro-pyrimidine-2,4-diamine (1.45 g, 10 mmol) in ethanol (50 mL) at room temperature. The reaction mixture was stirred at room temperature for 5 minutes. It was concentrated and partitioned between ethyl acetate and aqueous NaHCO₃ (50 mL, 1:1). The organic phase was washed with brine, dried (MgSO₄), filtered and concentrated under reduced pressure. The residue was triturated with EtOAc to provide the titled compound (1.35 g).

### EXAMPLE 129B

### N′-Benzyl-5-bromo-pyrimidine-2,4,6-triamine

The mixture of Example 129A (112 mg, 0.5 mmol), benzylamine (218 μL, 1.0 mmol) in ethanol (2 mL) was heated in a microwave oven at 150° C. for 90 minutes. It was then partitioned between ethyl acetate and brine (50 mL, 1:1). The organic phase was washed with brine, dried (MgSO₄), filtered and concentrated under reduced pressure. The residue was triturated with acetonitrile to provide the titled compound (72 mg).

### EXAMPLE 129C

### N′-Benzyl-5-(4-nitro-phenyl)-pyrimidine-2,4,6-triamine

The mixture of Example 129B (59 mg, 0.2 mmol), boronic acid (37 mg, 0.22 mmol), PdCl₂(Ph₃P)₂ (3 mg) and Na₂CO₃ (25 mg, 0.24 mmol) in a mixed solvent (1 mL) aof DME:H₂O:EtOH (7:3:2) was heated in a microwave oven at 150° C. for 15 minutes. It was then partitioned between ethyl acetate and aqueous NaHCO₃ (50 mL, 1:1). The organic phase was washed with brine, dried (MgSO₄), filtered and concentrated under reduced pressure. The residue was purified by flash chromatography on silica gel column with ethyl acetate/methanol (10/1) to provide the titled compound (22 mg).

### EXAMPLE 129D

### N′-Benzyl-5-[4-(4-methanesulfonyl-benzylaniino)-phenyl]-pyrmdine-2,4,6-trianmine

The titled compound was synthesized according to the procedure described in Example 1, substituting Example 129C for Example 1B and 4-methanesulfonyl-benzaldehyde for 4-chlorobenzaldehyde. ¹H NMR (300 MHz, DMSO-d₆) δ 7.89 (d, 2H, J=8.5 Hz), 7.64 (d, 2H, J=8.5 Hz), 7.30-7.15(m, 5H), 6.92 (d, 2H, J=8.5 Hz), 6.65 (d, 2H, J=8.5 Hz), 6.49 (t, 1H, J=5.8 Hz), 5.44 (s, 2H), 5.27-5.18(m, 1H), 4.78 (s, 2H), 4.45-4.35 (m, 4H), 3.18 (s, 3H); MS (ESI) positive ion 475 (M+H)⁺; negative ion 473 (M−H)⁻.

### EXAMPLE 130

### 6-cyclobutyl-5-(4-{[4-(methylsulfonyl)benzyl]amino}phenyl)pyrimidine-2,4-diamine

### EXAMPLE 130A

### 5-(4-Amino-phenyl)6-cyclobutyl-pyrimidine-2,4-diamine

The titled compound was prepared according to the procedure in Example 2A-C, substituting cyclobutane carbonyl chloride for benzyloxyacetyl chloride used in Example 2.

### EXAMPLE 130B

### 6-Cyclobutyl-5-(4-{[4-(methylsulfonyl)benzyl]amino}phenyl)pyrimidine-2,4-diamine

To a solution of 5-(4-amino-phenyl)-6-cyclobutyl-pyrimidine-2,4-diamine from Example 130A (170 mg, 0.67 mmol) in MeOH (6.7 mL) was added 4-methylsulfonyl benzaldehyde (135 mg, 0.73 mmol). To the stirred solution at room temperature was added NaCNBH₃ (55 mg, 0.86 mmol) and glacial HOAc (0.19 mL). The reaction stirred overnight and was concentrated and the residue taken up in saturated NaHCO₃ solution (10 mL). It was washed with EtOAc (2×10 mL) and the combined organic layers were washed with brine (10 mL), dried over MgSO₄ and concentrated. Purified via column chromatography, eluting 95% CH₂Cl₂/MeOH (0.5% NH4OH). The resulting foam was triturated with Et₂O and filtered to give an off-white solid (40 mg, 14% yield). ¹H NMR (300 MHz, DMSO-d₆) δ 7.89-7.91 (d, J=8.48 Hz, 2H), 7.64-7.67 (d, J=8.14 Hz, 2H), 6.78-6.81 (d, J=8.47 Hz, 2H), 6.60-6.63 (d, J=8.47 Hz, 2H), 6.45-6.49 (t, J=5.76 Hz, 6.11 Hz, 1H), 5.74 (s, 2H), 5.26 (s, 2H), 4.39-4.41 (d, J=5.76 Hz, 2H), 3.20 (s, 3H), 3.12-3.18 (m, 1H), 2.18-2.30 (m, 2H), 1.62-1.76 (m, 4H). MS (ESI) positive ion 424 (M+H)⁺; negative ion 422 (M−H)⁻.

### EXAMPLE 131

### 6-cyclopropyl-5-(4-{[4-(methylsulfonyl)benzyl]amino}phenyl)pyrimidine-2,4-diamine

### EXAMPLE 131A

### 5-(4-Amino-phenyl)6-cyclopropyl-pyrimidine-2,4-diamine

The titled compound was prepared according to the procedure in Example 2A-C, substituting cyclopropane carbonyl chloride for benzyloxyacetyl chloride used in Example 2.

### EXAMPLE 131B

### 6-Cyclopropyl-5-(4-{[4-(methylsulfonyl)benzyl]amino}phenyl)pyrimidine-2,4-diamine

To a solution of 5-(4-amino-phenyl)-6-cyclopropyl-pyrimidine-2,4-diamine from Example 131A (150 mg, 0.622 mmol) in MeOH (6.2 mL) was added 4 methanesulfonyl benzaldehyde (126 mg, 0.684 mmol). The NaCNBH₃ (51 mg, 0.809 mmol) and glacial acetic acid (0.18 mL) were added and the reaction stirred at room temperature for 6 h. It was the concentrated under reduced pressure and taken up in saturated NaHCO₃ solution (10 mL), which was washed with EtOAc (2×10 mL). The combined organic layers were washed with brine (10 mL), dried over MgSO₄ and concentrated. The crude product was purified via column chromatography, eluting 95% CH₂Cl₂/MeOH (0.5% NH4OH), then triturated with Et₂O to give a white solid (55 mg, 22% yield). ¹H NMR (300 MHz, DMSO-d₆) δ 7.88-7.91 (d, J=8.48 Hz, 2H), 7.64-7.66 (d, J=8.47 Hz, 2H), 6.91-6.94 (d, J=8.48 Hz, 2H), 6.63-6.66 (d, J=8.47 Hz, 2H), 6.46-6.50 (t, J=6.10 Hz, 1H), 5.59 (s, 2H), 5.25 (s, 2H), 4.39-4.41 (d, J=5.76 Hz, 2H), 3.19 (s, 3H), 1.45-1.53 (m, 1H), 0.80-0.85 (m, 2H), 0.56-0.62 (m, 2H). MS (ESI) positive ion 410 (M+H)⁺; negative ion 408 (M−H)⁻.

### EXAMPLE 132

### 6-(2,3-dihydro-1,4-benzodioxin-2-yl)-5-(4-{[4-(methylsulfonyl)benzyl]amino}phenyl)pyrimidine-2,4-diamine

The titled compound was prepared according to the procedure described in Examples 45 and 130, substituting 1,4-benzodioxan-2-carboxylic acid for 3-(methylbutoxy)acetic acid used in Example 45A (15% yield). ¹H NMR (300 MHz, DMSO-d₆) δ 7.85-7.89 (m, J=8.14 Hz, 4H), 7.56-7.64 (m, J=8.48 Hz, 4H), 6.97 (d, J=8.48 Hz, 2H), 6.83 (d, J=2.03 Hz, 2H), 6.62 (d, J=8.48 Hz, 2H), 6.05 (br s, 1H), 5.44 (t, J=5.59 Hz, 2H), 4.62 (d, J=5.76 Hz, 2H), 4.39 (m, J=6.1 Hz, 3H), 3.19 (s, 3H). MS (E126I): 504.1 (M+H), 502.0 (M−H).

### EXAMPLE 133

### 6-[(1R)-1-(benzyloxy)ethyl]-5-(4-{[4-(methylsulfonyl)benzyl]amino}phenyl)pyrimidine-2,4-diamine

The title compound was synthesized according to the procedure described in Example 2, substituting 4-methanesulfonyl-benzylamine for 4-chlorobenzylamine and (R)-2-benzyloxy-propionyl chloride for benzyloxy-acetyl chloride. ¹H NMR (300 MHz, DMSO-d₆) δ 7.90 (d, 2H, J=8.5 Hz), 7.65 (d, 2H, J=8.5 Hz), 7.30-7.15(m, 5H), 6.88-6.77 (m, 2H), 6.59 (d, 2H, J=8.5 Hz), 6.49 (t, 1H, J=5.8 Hz), 5.90 (s, 2H), 5.40 (bs, 2H), 4.39(d, 2H, J=5.8 Hz), 4.25-4.04 (m, 3H), 3.19 (s, 3H); MS (ESI) positive ion 504 (M+H)⁺; negative ion 502 (M−H)⁻.

### EXAMPLE 134

### 6-tert-butyl-5-(4-{[4-(methylsulfonyl)benzyl]amino}phenyl)pyrimidine-2,4-diamine

The titled compound was prepared according to the procedure described in Example 2, substituting 2,2,-dimethyl-propionyl chloride for benzyloxyacetyl chloride, and 4-methanesulfonyl benzaldehyde for 4-chlorobenzaldehyde used in Example 2 (54% yield). ¹H NMR (300 MHz, DMSO-d₆) δ 10.48 (s, 1H), 8.15 (s, 2H), 7.90 (d, J=8.48 Hz, 2H), 7.65 (d, J=8.5 Hz, 2H), 6.90 (d, J=8.1 Hz, 2H), 6.66 (d, J=8.5 Hz, 2H), 6.19 (s, 2H), 4.42 (d, J=5.8 Hz, 2H), 3.2 (s, 3H), 1.06 (s, 9H). MS (ESI) positive ion 426 (M+H)⁺; negative ion 424-(M−H)⁻.

### EXAMPLE 135

### 6-cyclopentyl-5-(4-{[4-(methylsulfonyl)benzyl]amino}phenyl)pyrimidine-2,4-diamine

### EXAMPLE 135A

### 5-(4-Amino-phenyl)6-cyclopentyl-pyrimidine-2,4-diamine

The titled compound was prepared according to the procedure in Example 2A-C, substituting cyclopentane carbonyl chloride for benzyloxyacetyl chloride in Example 2.

### EXAMPLE 135B

### 6-cyclopentyl-5-(4-{[4-(methylsulfonyl)benzyl]amino}phenyl)pyrimidine-2,4-diamine

To a solution of 5-(4-amino-phenyl)-6-cyclopentyl-pyrimidine-2,4-diamine from Example 135A (20 mg, 0.074 mmol) in MeOH (0.7 mL) was added 4-methanesulfonyl benzaldehyde (14 mg, 0.078 mmol). The NaCNBH₃ (6 mg, 0.096 mmol) and glacial acetic acid (0.02 mL) were added after 1.5 h and the reaction stirred at room temperature for 15 h. It was the concentrated under reduced pressure and taken up in saturated NaHCO₃ solution (5 mL), which was washed with EtOAc (2×5 mL). The combined organic layers were washed with brine (5 mL), dried over MgSO₄ and concentrated. The crude product was purified via column chromatography, eluting 95% CH₂Cl₂/MeOH (0.5% NH4OH), then triturated with Et₂O to give a pale yellow solid (12 mg, 40% yield). ¹H NMR (300 MHz, DMSO-d₆) δ 7.88-7.91 (d, J=8.14 Hz, 2H), 7.64-7.67 (d, J=8.47 Hz, 2H), 6.83-6.86 (d, J=8.48 Hz, 2H), 6.61-6.64 (d, J=8.82 Hz, 2H), 6.44-6.48 (t, J=6.10 Hz, 1H), 5.66 (s, 2H), 5.21 (s, 2H), 4.39-4.41 (d, J=6.10; Hz, 2H), 3.19 (s, 3H), 2.61-2.66 (m, 1H), 1.53-1.63 (m, 6H), 1.37-1.41 (m, 2H).MS (ESI) positive ion 438 (M+H)⁺; negative ion 436 (M−H)⁻.

### EXAMPLE 136

### 6-cyclohexyl-5-(4-{[4-(methylsulfonyl)benzyl]amino}phenyl)pyrimidine-2,4-diamine

### EXAMPLE 136A

### 5-(4-Amino-phenyl)6-cyclohexyl-pyrimidine-2,4-diamine

The titled compound was prepared according to the procedure in Example 2A-C, substituting cyclohexane carbonyl chloride for benzyloxyacetyl chloride in Example 2.

### EXAMPLE 136B

### 6-cyclohexyl-5-(4-{[4-(methylsulfonyl)benzyl]amino}phenyl)pyrimidine-2,4-diamine

To a solution of 5-(4-amino-phenyl)-6-cyclohexyl-pyrimidine-2,4-diamine from Example 136A (125 mg, 0.442 mmol) in MeOH (4.4 mL) was added 4-methanesulfonyl benzaldehyde (85 mg, 0.464 mmol). The NaCNBH₃ (36 mg, 0.573 mmol) and glacial acetic acid (0.03 mL) were added after 1 h and the reaction stirred at room temperature for 4 h. It was the concentrated under reduced pressure and taken up in saturated NaHCO₃ solution (5 mL), which was washed with EtOAc (2×5 mL). The combined organic layers were washed with brine (5 mL), dried over MgSO₄ and concentrated. The crude product was purified via column chromatography, eluting 95% CH₂Cl₂/MeOH (0.5% NH4OH), then triturated with MeOH to give a white solid (30 mg, 15% yield). ¹H NMR (300 MHz, DMSO-d₆) δ 7.88-7.91 (d, J=8.14 Hz, 2H), 7.64-7.67 (d, J=8.47 Hz, 2H), 6.82-6.85 (d, J=8.14 Hz, 2H), 6.62-6.65 (d, J=8.48 Hz, 2H), 6.43-6.47 (t, J=5.77 hz, 6.10 Hz, 1H), 5.66 (s, 2H), 5.21 (s, 2H), 4.39-4.41 (d, J=5.76 Hz, 2H), 3.19 (s, 3H), 2.17-2.25 (m, 1H), 1.47-1.65 (m, 7 H), 0.89-1.15 (m, 3H). MS (ESI) positive ion 452 (M+H)⁺; negative ion 450 (M−H)⁻.

### EXAMPLE 137

### 6-(2,3-dihydro-1-benzofuran-2-yl)-5-(4-{[4-(methylsulfonyl)benzyl]amino}phenyl)pyrimidine-2,4-diamine

The title compound was synthesized according to the procedure described in Example 2, substituting 4-methanesulfonyl-benzylamine for 4-chlorobenzylamine and 2,3-dihydro-benzofuran-2-carbonyl chloride for benzyloxy-acetyl chloride. ¹H NMR (300 MHz, DMSO-d₆) δ 7.89 (d, 2H, J=8.5 Hz), 7.64 (d, 2H, J=8.5 Hz), 7.17-6.51 (m, 9H), 5.91 (s, 2H), 5.41 (bs, 2H), 5.28-5.20 (m, 1H), 4.40 (d, 2H, J=6.1 Hz), 3.53-3.09 (m, 2H), 3.18 (s, 3H); MS (ESI) positive ion 488 (M+H)⁺; negative ion 486 (M−H)⁻.

### EXAMPLE 138

### 6-(cyclopentylmethyl)-5-(4-{[4-(methylsulfonyl)benzyl]amino}phenyl)pyrimidine-2,4-diamine

The title compound was synthesized according to the procedure described in Example 2, substituting 4-methanesulfonyl-benzylamine for 4-chlorobenzylamine and cyclopentyl-acetyl chloride for benzyloxy-acetyl chloride. ¹H NMR (300 MHz, DMSO-d₆) δ 7.89 (d, 2H, J=8.5 Hz), 7.65 (d, 2H, J=8.5 Hz), 6.84 (d, 2H, J=8.5 Hz), 6.63 (d, 2H, J=8.5 Hz), 6.46 (t, 1H, J=6.1 Hz), 5.91 (bs, 2H), 5.40 (bs, 2H), 4.41(d, 2H, J=6.1 Hz), 3.19 (s, 3H), 2.18-2.08 (m, 3H), 1.59-0.85 (m, 8H); MS (ESI) positive ion 452 (M+H)⁺; negative ion 450 (M−H)⁻.

### EXAMPLE 139

### 5-(4-{[4-(methylsulfonyl)benzyl]amino}phenyl)-6-tetrahydro-2H-pyran-4-ylpyrimidine-2,4-diamine

### EXAMPLE 139A

### 5-(4-Amino-phenyl)6-(tetrahydro-pyran-4-yl)-pyrimidine-2,4-diamine

The titled compound was prepared according to the procedure in Example 2A-C, substituting 4-tetrahydropyranyl carbonyl chloride for benzyloxyacetyl chloride in Example 2.

### EXAMPLE 139B

### 5-(4-{[4-(methylsulfonyl)benzyl]amino}phenyl)-6-tetrahydro-2H-pyran-4-ylpyrimidine-2,4-diamine

To a solution of 5-(4-amino-phenyl)-6-(tertahydro-pyran-4-yl)-pyrimidine-2,4-diamine from Example 139A (90 mg, 0.316 mmol) in MeOH (3.1 mL) was added 4-methanesulfonyl benzaldehyde (61 mg, 0.332 mmol). The NaCNBH₃ (26 mg, 0.411 mmol) and glacial acetic acid (0.09 mL) were added and the reaction stirred at room temperature for 1 h. It was the concentrated under reduced pressure and taken up in saturated NaHCO₃ solution (10 mL), which was washed with EtOAc (2×10 mL). The combined organic layers were washed with brine (5 mL), dried over MgSO₄ and concentrated. The crude product was purified via column chromatography, eluting 95% CH₂Cl₂/MeOH (0.5% NH4OH to give a white solid (30 mg, 21% yield). ¹H NMR (300 MHz, DMSO-d₆) δ 7.88-7.91 (d, J=8.47 Hz, 2H), 7.64-7.67 (d, J=8.14 Hz, 2H), 6.82-6.85 (d, J=8.47 Hz, 2H), 6.62-6.65 (d, J=8.77 Hz, 2H), 6.44-6.49 (t, J=6.10 Hz, 1H), 5.72 (s, 2H), 5.26 (s, 2H), 4.39-4.41 (d, J=5.76 Hz, 2H), 3.76-3.81 (dd, J₁=3.73 Hz, J₂=3.39 Hz, 2H), 3.19 (s, 3H), 3.18 (m, 1H), 3.02-3.10 (m, 2H), 1.73-1.87 (m, 2 H), 1.28-1.32 (m, 2H). MS (ESI) positive ion 454 (M+H)⁺; negative ion 452 (MH)⁻.

### EXAMPLE 140

### 6-ethyl-5-{3-ethyl-1-[4-(methylsulfonyl)benzyl]-1H-indol-5-yl}pyrimidine-2,4-diamine

### EXAMPLE 140A

### 6-Ethyl-5-(4-hydrazino-phenyl)-pyrimidine-2,4-diamine

To a solution of 2.00 g (8.72 mmol) of 5-(4-amino-phenyl)-6-ethyl-pyrimidine-2,4-diamine from Example 1C in 30 mL of 12 M HCl cooled with an ice-salt bath was added a solution of 720 mg (10.4 mmol) of NaNO₂ dissolved in a minimum amount of H₂O. The solution was stirred for 20 min, then a white suspension of 4.7 g (20.8 mmol) of SnCl₂.2H₂O was added. After stirring for 40 min, a fine precipitate was collected and stirred into 30 mL of 25% (w/v) NaOH_{(aq.)}. The precipitate was collected, washed with H₂O until pH=8 in the filter funnel, dried partially on the filter, then the remaining water was coevaporated with EtOH in vacuo to give 1.32 g (62%) of a yellow solid.¹H NMR (300 MHz, d₆-DMSO) δ 6.90 (m, 2H), 6.83 (m, 2H), 6.74 (s, 1H), 5.74 (bs, 2H), 5.34 (s, 2H), 3.94 (s, 2H), 2.13 (q, 2H, J=7.5 Hz), 0.95 (t, 3H, J=7.5 Hz); MS (ESI) m/z =245 (M+H)⁺.

### EXAMPLE 140B

### 6-Ethyl-5-(3-ethyl-1H-indol-5-yl)-pyrimidine-2,4-diamine

To a solution of 100 mg (0.409 mmol) of 6-ethyl-5-(4-hydrazino-phenyl)-pyrimidine-2,4-diamine from Example 140A in 2 mL of glacial acetic acid was added 30 mg (0.42 mmol) of butyraldehyde. The solution was stirred at ambient temperature for 2.5 h, then 250 mg (1.31 mmol) of p-toluenesulfonic acid monohydrate was added. The solution was heated at 100° C., then diluted with 20 mL of 7.5M NH₄OH. The product was extracted with ethyl acetate (3×5 mL), then the combined ethyl acetate layers were back extracted with water (1×5 mL) and brine (1×5 mL), dried over MgSO₄, filtered, and concentrated to an oil. This was partially purified via reverse-phase HPLC, eluting with a 5-100% CH₃CN gradient in 95% aqueous 0.01M NH₄OAc/5% CH₃CN to give a red glass. This was further purified over a 5 mL silica gel column, eluting with 95:4.5:0.5 ethyl acetate: methanol: 15M NH₄OH to give 25 mg (22%) of the titled compound as a yellow foam. ¹H NMR (300 MHz, d₆-DMSO) δ 10.79 (d, 1H, J=2.0 Hz), 7.38 (d, 1H, J=8.3 Hz), 7.27 (m, 1H), 7.12 (m, 1H), 6.84 (dd, 1, J=8.3, 1.5 Hz), 5.75 (s, 2H), 5.29 (bs, 2H), 2.69 (m, 2H), 2.13 (m, 2H), 1.25 (t, 3H, J=7.5 Hz), 0.96 (t, 3H, J=7.6 Hz); MS (ESI) m/z=282 (M+H)⁺.

### EXAMPLE 140C

### 6-Ethyl-5-[3-ethyl-1-(4-methanesulfonyl-benzyl)-1H-indol-5-yl]-pyrimidine-2,4-diamine

To a solution of 25 mg (0.089 mmol) of 6-ethyl-5-(3-ethyl-1H-indol-5-yl)-pyrimidine-2,4-diamine from Example 140B in 0.5 mL of DMF was added 88 mL (0.088 mmol) of 1.0 M potassium tert-butoxide in tert-butanol. The reaction was stirred for 1 min, then 22 mg (0.088 mmol) of 4-(methylsulfonyl)benzylbromide was added, and the reaction was complete within 2 min. The solution was diluted with 1 mL of CH₃OH, then injected onto a reverse phase HPLC column, eluting with 0-70% CH₃CN in 0.1% aq. CF₃CO₂H. The product containing fractions were concentrated to a solid, and this was stirred with 1 mL of 5% NH₄OH, then extracted 2×2 mL ethyl acetate. The ethyl acetate layers were dried over MgSO₄, filtered, and concentrated to give 12 mg (30%) of the titled compound as a white foam. ¹H NMR (300 MHz, d₆DMSO) δ 7.89 (m, 2H), 7.52 (m, 2H), 7.48 (d, 1H, J=8.5 Hz), 7.32 (m, 2H), 6.90 (dd, 1H, J=8.5, 1.4 Hz), 5.78 (s, 2H), 5.49 (s, 2H), 5.32 (s, 2H), 3.17 (s, 3H), 2.71 (q, 2H, J=7.5 Hz), 2.11 (q, 2H, J=7.5 Hz), 1.27 (t, 3H, J=7.5 Hz), 0.96 (t, 3H, J=7.5 Hz); MS (ESI) m/z=450 (M+H)⁺.

### EXAMPLE 141

### 5-(4-{[4-(methylsulfonyl)benzyl]amino}phenyl)-6-[(2S)-tetrahydrofuran-2-yl]pyrimidine-2,4-diamine

The titled compound was prepared according to the procedure described in Example 2, substituting (S)-(−)-tetrahydro-furan-2-carbonyl chloride for benzyloxyacetyl chloride, and 4-methanesulfonyl benzaldehyde for 4-chlorobenzaldehyde used in Example 2 (57% yield). ¹H NMR (300 MHz, DMSO-d₆) δ 7.90 (d, J=8.1 Hz, 2H), 7.65 (d, J=8.1 Hz, 2H), 6.86 (dd, J₁=20.8, J=6.6 Hz, 2H), 6.62 (d, J=8.1 Hz, 2H), 6.50 (t, J=6.5 Hz, 1H), 5.82 (s, 2H), 5.44 (s, 2H), 4.40 (d, J=6.1 Hz, 2H), 4.32 (t, J=6.1 Hz, 1H), 3.80 (m, 1H), 3.57 (m, 1H), 3.10 (s, 3H), 1.96(m, 2H), 1.75 (m, 2H). MS (ESI) positive ion 440 (M+H)⁺; negative ion 438 (M−H)⁻. [α]²⁰=+17.6 (MeOH, C=1).

### EXAMPLE 142

### 5-(4-{[4-(methylsulfonyl)benzyl]amino}phenyl)-6-[(2R)-tetrahydrofuran-2-yl]pyrimidine-2,4-diamine

The titled compound was prepared according to the procedure described in Example 2, substituting (R)-tetrahydro-furan-2-carbonyl chloride for benzyloxyacetyl chloride, and 4-methanesulfonyl benzaldehyde for 4-chlorobenzaldehyde used in Example 2 (54% yield). ¹H NMR (300 MHz, DMSO-D₆) δ 7.90 (d, J=8.1 Hz, 2H), 7.65 (d, J=8.1 Hz, 2H), 6.86 (dd, J₁=20.8, J=6.6 Hz, 2H), 6.62 (d, J=8.1 Hz, 2H), 6.50 (t, J=6.5 Hz, 1H), 5.82 (s, 2H), 5.44 (s, 2H), 4.40 (d, J=6.1 Hz, 2H), 4.32 (t, J=6.1 Hz, 1H), 3.80 (m, 1H), 3.57 (m, 1H), 3.10 (s, 3H), 1.96(m, 2H), 1.75 (m, 2H). MS (ESI) positive ion 440 (M+H)⁺; negative ion 438 (M−H)⁻. [α]²⁰=−17.6 (MeOH, C=1)).

### EXAMPLE 143

### 6-ethyl-5-{2-methyl-1-[4-(methylsulfonyl)benzyl]-1H-benzimidazol-5-yl}pyrimidine-2,4-diamine

### EXAMPLE 143A

### 6-Ethyl-5-[4-(4-methanesulfonyl-benzylamino)-3-nitro-phenyl]-pyrimidine-2,4-diamine

To a solution of 2,4-diamino-5-(4-chloro-3-nitrophenyl)-6-ethylpyrimidine (Griffin, et. al, J. Chem. Soc. Perkin Trans I, 1985, 2267-2276.) (176 mg, 0.6 mmol) in NMP (3.0 mL) was added 4-methanesulfonyl benzylamine hydrochloride (266 mg, 1.2 mmol) and diisopropylethylamine (0.42 mL, 2.4 mmol). The reaction was heated at 190° C. for 45 minutes in a Personal Chemistry Optimizer Micro Wave reactor. The reaction was diluted with saturated NaHCO₃ solution (15 mL) and washed with EtOAc (3×10 mL). The combined organic layers were washed with H₂O (2×10 mL), brine (10 mL), dried over MgSO₄ and concentrated. Purified via reverse phase HPLC (0-70% aqueous NH₄OAc/CH₃CN) to give a yellow solid (50 mg, 19% yield).

### EXAMPLE 143B

### 5-[3-Amino-4-(4-methanesulfonyl-benzylamino)-phenyl]-6-ethyl-pyrimidine-2,4-diamine

To a solution of 6-ethyl-5-[4-(4-methanesulfonyl-benzylamino)-3-nitro-phenyl]-pyrimidine-2,4-diamine (100 mg, 0.226 mg) from Example 143A in isopropyl alcohol (1.9 mL) and H₂O (0.4 mL) was added iron (75 mg, 1.35 mmol) and NH₄Cl (16 mg, 0.294 mmol). The reaction was heated to reflux for 30 minutes, then cooled to room temperature and filtered through a plug of Celite, rinsed with MeOH and concentrated. The residue was taken up in saturated NaHCO₃ (10 mL) solution and washed with EtOAc (2×15 mL). The combined organic layers were washed with brine (10 mL), dried over MgSO₄, and concentrated. Triturated with CH₃CN and filtered to give a yellow solid (50 mg, 55% yield).

### EXAMPLE 143C

### 6-ethyl-5-{2-methyl-1-[4-(methylsulfonyl)benzyl]-1H-benzimidazol-5-yl}pyrimidine-2,4-diamine

A solution of 5-[3-amino-4-(4-methanesulfonyl-benzylamino)-phenyl]-6-ethyl-pyrimidine-2,4-diamine from Example 143B (55 mg, 0.133 mmol) in 0.3 mL of glacial acetic acid was heated to reflux for 3 h. The reaction was cooled to room temperature and then made basic by the addition of saturated NaHCO₃ solution. The aqueous solution was extracted with EtOAc (3×15 mL) and the combined organic layers were washed with brine (10 mL), dried over MgSO₄ and concentrated. The product was triturated with MeOH and filtered to give an orange solid (5 mg, 9% yield). ¹H NMR (300 MHz, DMSO-d₆) δ 7.90-7.92 (d, J=8.28 Hz, 2H), 7.49-7.52 (d, J=8.28 Hz, 1H), 7.43-7.45 (d, J=8.28 Hz, 2H), 7.33 (s, 1H), 6.94-6.96 (d, J=9.52 Hz, 1H), 5.77 (s, 2H), 5.62 (s, 2H), 5.34 (s, 2H), 3.19 (s, 3H), 2.54 (s, 3H), 2.08-2.13 (q, J=7.67, 7.36, 7.67 Hz, 2H), 0.93-0.97 (t, J=7.37, 7.67 Hz, 3H). MS (ESI) positive ion 437 (M+H)⁺; negative ion 435 (M−H)⁻.

### EXAMPLE 144

### 5-(4-{[4-(methylsulfonyl)benzyl]amino}phenyl)-N-4-(2-phenylethyl)pyrimidine-2,4,6-triamine

The title compound was synthesized according to the procedure described in Example 129, substituting phenylethylamine for benzylarnine. ¹H NMR (300 MHz, DMSO-d₆) δ 7.91 (d, 2H, J=8.5 Hz), 7.66 (d, 2H, J=8.5 Hz), 7.25-7.08(m, 5H), 6.80 (d, 2H, J=8.5 Hz), 6.60 (d, 2H, J=8.5 Hz), 6.48 (t, 1H, J=5.8 Hz), 5.46 (s, 2H), 4.73 (s, 2H), 4.60-4.52(m, 1H), 4.39 (d, 2H, J=5.8 Hz), 3.43-3.30 (m, 2H), 3.19 (s, 3H), 2.70-2.65 (m, 2H); MS (ESI) positive ion 489 (M+H)⁺; negative ion 487 (M−H)⁻.

### EXAMPLE 145

### 5-{2-cyclopentyl-1-[4-(methylsulfonyl)benzyl]-1H-benzimidazol-5-yl}-6-ethylpyrimidine-2,4-diamine

### EXAMPLE 145A

### Cyclopentanecarboxylic acid [5-(2,4-diamino-6-ethyl-pyrimidine-5-yl)-2-(4-methanesulfonyl-benzylamino-phenyl}-amide

To a solution of 5-[3-amino-4-(4-methanesulfonyl-benzylamino)-phenyl]-6-ethyl-pyrimidine-2,4-diamine from Example 143B (50 mg, 0.113 mmol) in THF (1.1 mL) was added cyclopentanecarbonyl chloride (0.014 mL, 0.113 mmol). After 1 h at room temperature, the reaction is concentrated in vacuo, taken up in saturated NaHCO₃ solution, and filtered. A yellow solid is isolated (55 mg, 95% yield) as the titled compound.

### EXAMPLE 145B

### 5-{2-cyclopentyl-1-[4-(methylsulfonyl)benzyl]-1H-benzimidazol-5-yl}-6-ethylpyrimidine-2,4-diamine

Cyclopentanecarboxylic acid [5-(2,4-diamino-6-ethyl-pyriridine-5-yl)-2-(4-methanesulfonyl-benzylamino-phenyl]-amide (55 mg, 0.108 mmol) was dissolved in polyphosphoric acid (0.5 mL) and heated to 150° C. for 1 h. It was cooled to room temperature, diluted with H₂O, and basified with 50% NaOH solution to pH=10. It was diluted with H₂O and filtered. The solid was triturated with CH₃CN and filtered. A light brown solid is isolated (20 mg, 38% yield). ¹H NMR (300 MHz, DMSO-d₆) δ 7.89-7.92 (d, J=8.12 Hz, 2H), 7.45-7.48 (d, J=8.14 Hz, 1H), 7.41 (s, 1H), 7.37-7.40 (d, J=8.48 Hz, 2H), 6.94-6.97 (dd, J=1.36, 6.48, 1.36 Hz, 1H), 6.23 (s, 2H), 5.68 (s, 2H), 5.63 (s, 2H), 3.32-3.4 (m, 1H), 3.20 (s, 3H), 2.09-2.17 (q, 2H), 1.67-2.03 (m, 8 H), 0.94-0.99 (t, J=7.48, 7.97 Hz, 3 H). MS(ESI) positive ion 491 (M+H)⁺; negative ion 489 (M−H)⁻.

### EXAMPLE 146

### 6-ethyl-5-{1-[4-(methylsulfonyl)benzyl]-2-phenyl-1H-benzimidazol-5-yl}pyrimidine-2,4-diamine

The titled compound was prepared according to the procedure in Example 145, substituting benzoyl chloride (0.013 mL, 0.113 mmol) for cyclopentanecarbonyl chloride. A light brown solid was isolated (33 mg, 58% yield over 2 steps). ¹H NMR (300 MHz, DMSO-d₆) δ 7.86-7.89 (d, J=8.14 Hz, 2H), 7.70-7.73 (m, 2H), 7.51-7.55 (m, 4H), 7.32-7.35 (d, J=8.48 Hz, 2H), 7.04-7.06 (d, J=9.83 Hz, 2H), 5.96 (s, 2H), 5.72 (s, 2H), 5.58 (s, 2H), 3.19 (s, 3H), 2.12-2.19 (q, J=7.49, 7.76, 7.80 Hz, 2H), 0.96-1.01 (t, J=7.46, 7.80 Hz, 3H). MS (ESI) positive ion 499 (M+H)⁺; negative ion 497 (M−H)⁻.

### EXAMPLE 147

### 6-ethyl-5-(3-methyl-4-{[4-(methylsulfonyl)benzyl]amino}phenyl)pyrimidine-2,4-diamine

### EXAMPLE 147A

### 5-Bromo-6-ethyl-pyrimidine-2,4-diamine

To 6-ethyl-pyrimidine-2,4-diamine (2 g, 14 mmol, Marianne, R. L.; Malcolm, S. F. G. J. Chem. Res. Synop. 2002, 10, 482-484.) in EtOH (10 mL) was added Br₂ (2.3 g, 14 mmol). The precipitates were collected after 1 h to give the titled 5-bromo-6-ethyl-pyrimidine-2,4-diamine (1 g, 31%).

### EXAMPLE 147B

### 2-Methyl-4-(4,4,5,5-tetramethyl-[1,3,2]dioxaborolan-2-yl)-phenylamine

A mixture of 4-bromo-2-methyl-phenylamine (372 mg, 2 mmol), bis(pinacolato)diboron (508 mg, 2 mmol), PdCl₂dppf (82 mg, 0.1 mmol), and KOAc (493 mg, 5 mmol) in 1,4-dioxane was heated at 92° C. for 3 h. The reaction mixture was partitioned between EtOAc and water and the organic extract was concentrated and purified by column chromatography to give the titled 2-methyl-4-(4,4,5,5-tetramethyl-[1,3,2]dioxaborolan-2-yl)-phenylamine (350 mg, 76%).

### EXAMPLE 147C

### 5-(4-Amino-3-methyl-phenyl)-6-ethyl-pyrimidine-2,4-diamine

To a mixture of boronate from Example 147B (124 mg, 0.5 mmol), 5-bromo-6-ethyl-pyrimidine-2,4-diamine from Example 147A (88 mg, 0.4 mmol), Pd(Ph₃)₄ (23 mg, 0.02 mmol) in toluene (1 mL) was added EtOH (0.4 mL) and K₂CO₃aq (3 M, 0.5 mL, 1.5 mmol). The mixture was heated at 120° C. under microwave irradiation for 20 min. The mixture was extracted with EtOAc and the organic extract was concentrated and purified by column chromatography to give 5-(4-amino-3-methyl-phenyl)-6-ethyl-pyrimidine-2,4-diamine (70 mg, 72%).

### EXAMPLE 147D

### 6-ethyl-5-(3-methyl-4-{[4-(methylsulfonyl)benzyl]amino}phenyl)pyrimidine-2,4-diamine

The aniline from Example 147C (32 mg, 0.13 mmol) was dissolved in MeOH buffer (1 M, NaOAc/HOAC, pH4, 1.5 mL) followed by addition of 4-methanesulfonyl-benzaldehyde and NaBH₃CN (6.2 mg, 0.1 mmol). After 20 min, the reaction mixture was quenched with conc. HCl (aq, 3 drops) and purified by HPLC to give the titled compound (15 mg, 28%). ¹H NMR (500 MHz, DMSO-d₆) δ 7.88 (d, J=8.5 Hz, 2H), 7.66 (d, J=8.5 Hz, 2H), 6.80 (d, J=1H), 6.71 (dd, J=8.5,2 Hz, 1H), 6.39 (d, J=8.5 Hz, 1H), 5.86 (t, J=6.0 Hz, 1H), 5.76 (s, 2H), 5.31 (s, 2H), 4.47 (d, J=7.0 Hz, 2H), 3.19 (s, 3H), 2.20 (s, 3H), 2.10 (quartet, J=8.0 Hz, 2H), and 0.94 (d, J=8.0 Hz, 3H). MS (ESI) m/e positive ion 412 (M+H)⁺; negative ion 410 (M−H)⁺.

1. A compound of formula (I), 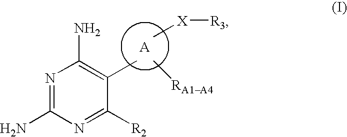
or a therapeutically suitable salt or prodrug thereof, wherein
A is a member selected from the group consisting of aryl, heteroaryl, and heterocycle;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylalkenyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl, and (R_{C}R_{D}N)sulfonylalkyl;
R₃ is a member selected from the group consisting of hydrogen, alkenyl, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxy, hydroxyalkyl, R_{G}R_{H}N—, and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, and (R_{E}R_{F}N)carbonyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl, and (R_{J}R_{K}N)carbonyl, or R_{G} and R_{H} together with the nitrogen atom to which they are attached form a heterocycle;
R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, haloalkoxy, haloalkyl, halogen, heteroaryl, heterocycle,hydroxy, hydroxyalkyl, nitro, R_{L}R_{M}N—, (R_{L}R_{M}N)alkyl, (R_{L}R_{M}N)carbonyl, and (R_{L}R_{M}N)sulfonyl;
R_{L} and R_{M} are each independently a member selected from the group consisting of hydrogen, alkoxycarbonyl, alkyl, alkylcarbonyl, alkoxysulfonyl, alkylsulfonyl, aryl, arylalkyl, and formyl, or R_{L} and R_{M} together with the nitrogen atom to which they are attached form a heterocycle;
X is a member selected from the group consisting of —O—, —NR_{N}—, —CH₂NH—; and
R_{N} is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle, heterocyclealkyl.

2. The compound according to claim 1, wherein
X is —NR_{N}—.

3. The compound according to claim 1, wherein
X is —NR_{N}—;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl;
R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, and (R_{E}R_{F}N)alkyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, and arylalkyl;
R_{G} and R_{H} are each independently a member selected from the group consisting of hydrogen, alkoxycarbonyl,-alkyl, alkylcarbonyl, arylalkyl, and arylcarbonyl;
R_{J} and R_{K} are each independently a member selected from the group consisting of hydrogen, alkoxycarbonyl, alkyl, and alkylcarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl;
R_{L} and R_{M} are each independently a member selected from the group consisting of hydrogen, alkoxycarbonyl, alkyl, and alkylcarbonyl.

4. The compound according to claim 1, wherein
X is —NR_{N}—;
R₂ is a member selected from the group consisting of alkenyl, alkenyloxyalkyl, alkoxyalkoxyalkyl, alkoxyalkyl, alkyl, arylalkoxyalkyl, arylalkyl, arylalkylalkenyl, arylalkylthioalkyl, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl;
R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (R_{G}R_{H}N)alkyl;
R_{C} and R_{D} are each independently a member selected from the group consisting of hydrogen, alkyl, aryl, arylalkyl, cycloalkylalkoxyalkyl, heteroarylalkyl, heterocyclealkyl, heterocyclealkoxyalkyl, and (R_{E}R_{F}N)alkyl;
R_{E} and R_{F} are each independently a member selected from the group consisting of hydrogen, alkoxycarbonyl,;
R_{G} and R_{H} are each independently a member selected from the group consisting of hydrogen, and alkoxycarbonyl;
R_{A1}, R_{A2}, R_{A3}, and R_{A4} are each independently a member selected from the group consisting of hydrogen, alkyl, aryl, and cycloalkyl.

5. The compound according to claim 1, wherein
X is —CH₂NH—.

6. The compound according to claim 1, wherein
X is —CH₂NH—;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl;
R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, and (R_{E}R_{F}N)alkyl, or R_{C} and R_{D} together with the nitrogen atom to which they are attached form a heterocycle;
R_{E} and R_{F} are each independently a member selected from the group consisting of hydrogen, alkoxycarbonyl, alkyl, and alkylcarbonyl;
R_{G} and R_{H} are each independently a member selected from the group consisting of hydrogen, alkoxycarbonyl, alkyl, alkylcarbonyl, arylalkyl, and arylcarbonyl;
R_{J} and R_{K} are each independently a member selected from the group consisting of hydrogen, alkoxycarbonyl, alkyl, and alkylcarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl;
R_{L} and R_{M} are each independently a member selected from the group consisting of hydrogen, alkoxycarbonyl, alkyl, and alkylcarbonyl.

7. The compound according to claim 1, wherein
X is —CH₂NH—;
R₂ is a member selected from the group consisting of alkenyl, alkenyloxyalkyl, alkoxyalkoxyalkyl, alkoxyalkyl, alkyl, arylalkoxyalkyl, arylalkyl, arylalkylalkenyl, arylalkylthioalkyl, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl;
R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (R_{G}R_{H}N)alkyl;
R_{C} and R_{D} are each independently a member selected from the group consisting of hydrogen, aryl, arylalkyl,-cycloalkylalkoxyalkyl, heteroarylalkyl, heterocyclealkyl, heterocyclealkoxyalkyl, and (R_{E}R_{F}N)alkyl;
R_{E} and R_{F} are each independently a member selected from the group consisting of hydrogen, and alkoxycarbonyl;
R_{G} and R_{H} are each independently a member selected from the group consisting of hydrogen, and alkoxycarbonyl;
R_{A1}, R_{A2}, R_{A3}, and R_{A4} are each independently a member selected from the group consisting of hydrogen, alkyl, aryl, and cycloalkyl.

8. The compound according to claim 1, wherein
X is —O—.

9. The compound according to claim 1, wherein
X is —O—;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkyl, alkylcarbonylalkyl, alkylsulfinylalkyl, alkylsulfonylalkyl, alkylthioalkyl, alkynylalkoxyalkyl, aryl, arylalkenyl, arylalkenyloxyalkyl, arylalkoxy, arylalkoxyalkyl, arylalkyl, arylalkylalkenyl, arylalkylthioalkyl, aryloxyalkyl, arylthioalkyl, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, hydroxyalkyl, R_{C}R_{D}N—, (R_{C}R_{D}N)alkyl, (R_{C}R_{D}N)carbonylalkenyl, and (R_{C}R_{D}N)carbonylalkyl;
R₃ is a member selected from the group consisting of hydrogen, alkenyl, alkoxy, alkoxyalkoxyalkyl, alkoxyalkyl, alkyl, alkylcarbonylalkyl, alkylsulfonylalkyl, alkylthioalkyl, alkynyl, aryl, arylalkoxyalkyl, arylalkyl, aryloxyalkyl, arylthioalkyl, cyanoalkyl, cycloalkenyl, cycloalkenylalkoxyalkyl, cycloalkenylalkyl, cycloalkyl, cycloalkylalkoxyalkyl, cycloalkylalkyl, cycloalkylalkylthioalkyl, cycloalkyloxyalkyl, cycloalkylthioalkyl, haloalkoxy, haloalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, R_{G}R_{H}N—, and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, aryl, arylalkoxyalkyl, arylalkyl, arylcarbonyl, cycloalkyl, cycloalkylalkyl, cycloalkylalkoxyalkyl, cycloalkylcarbonyl, formyl, heteroaryl, heteroarylalkyl, heteroarylcarbonyl, heterocycle, heterocyclealkyl, heterocyclealkoxyalkyl, heterocyclecarbonyl, (R_{E}R_{F}N)alkyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, and arylalkyl;
R_{G} and R_{H} are each independently a member selected from the group consisting of hydrogen, alkoxycarbonyl, alkyl, alkylcarbonyl, arylalkyl, and arylcarbonyl;
R_{J} and R_{K} are each independently a member selected from the group consisting of hydrogen, alkoxycarbonyl, alkyl, and alkylcarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, aryl, carboxy, carboxyalkyl, cyano, cyanoalkyl, cycloalkyl, haloalkyl, halogen, heteroaryl, heterocycle,hydroxy, hydroxyalkyl, nitro, R_{L}R_{M}N—, (R_{L}R_{M}N)alkyl, and (R_{L}R_{M}N)carbonyl;
R_{L} and R_{M} are each independently a member selected from the group consisting of hydrogen, alkoxycarbonyl, alkyl, and alkylcarbonyl.

10. The compound according to claim 1, wherein
X is —O—;
R₂ is a member selected from the group consisting of alkenyl, alkenyloxyalkyl, alkoxyalkoxyalkyl, alkoxyalkyl, alkyl, arylalkoxyalkyl, arylalkyl, arylalkylalkenyl, arylalkylthioalkyl, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxyalkyl, cycloalkylalkyl, heteroaryloxyalkyl, heterocycle, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, and (R_{C}R_{D}N)carbonylalkyl;
R₃ is a member selected from the group consisting of hydrogen, alkenyl, alkoxy, alkyl, arylalkoxyalkyl, arylalkyl, cycloalkylalkyl, heteroarylalkyl, heterocyclealkyl, and (R_{G}R_{H}N)alkyl;
R_{C} and R_{D} are each independently a member selected from the group consisting of hydrogen, alkyl, aryl, arylalkyl, cycloalkylalkoxyalkyl, heteroarylalkyl, heterocyclealkyl, heterocyclealkoxyalkyl, and (R_{E}R_{F}N)alkyl;
R_{E} and R_{F} are each independently a member selected from the group consisting of hydrogen, and alkoxycarbonyl;
R_{G} and R_{H} are each independently a member selected from the group consisting of hydrogen, and alkoxycarbonyl;
R_{A1}, R_{A2}, R_{A3}, and R_{A4} are each independently a member selected from the group consisting of hydrogen, alkyl, aryl, and cycloalkyl.

11. The compound according to claim 1, that is selected from the group consisting of
5-{4-[(4-chlorobenzyl)amino]phenyl}-6-ethylpyrimidine-2,4-diamine;
6-[(benzyloxy)methyl]-5-{4-[(4-chlorobenzyl)amino]phenyl}pyrimidine-2,4-diamine;
5-{4-[(4-chlorobenzyl)amino]phenyl}-6-(methoxymethyl)pyrimidine-2,4-diamine;
5-{4-[(4-chlorobenzyl)amino]phenyl }-6-{[(2-fluoro-3-methylbenzyl)oxy]methyl}pyrimidine-2,4-diamine;
5-{4-[(4-chlorobenzyl)amino]phenyl}-6-(3-phenylpropyl)pyrimidine-2,4-diamine;
4-({[4-(2,4-diamino-6-ethylpyrimidin-5-yl)phenyl]amino}methyl)benzonitrile;
5-{4-[(3,4-dichlorobenzyl)amino]phenyl}-6-ethylpyrimidine-2,4-diamine;
5-{4-[(4-chlorobenzyl)amino]phenyl}-6-(phenoxymethyl)pyrimidine-2,4-diamine;
5-{4-[(4-chlorobenzyl)amino]phenyl}-6-propylpyrimidine-2,4-diamine;
5-{4-[(4-chlorobenzyl)amino]phenyl}-6-{[(3-methylbenzyl)oxy]methyl}pyrimidine-2,4-diamine;
5-{4-[(4-chlorobenzyl)amino]phenyl}-6-{[(2-methoxybenzyl)oxy]methyl}pyrimidine-2,4-diamine;
5-{4-[(4-chlorobenzyl)amino]phenyl}-6-{[(3-methoxybenzyl)oxy]methyl}pyrimidine-2,4-diamine;
5-{4-[(4-chlorobenzyl)amino]phenyl}-6-{[(4-methoxybenzyl)oxy]methyl}pyrimidine-2,4-diamine;
5-{4-[(4-chlorobenzyl)amino]phenyl}-6-{[(2-fluorobenzyl)oxy]methyl}pyrimidine-2,4-diamine;
5-{4-[(4-chlorobenzyl)amino]phenyl}-6-{[(4-fluorobenzyl)oxy]methyl}pyrimidine-2,4-diamine;
5-{4-[(4-chlorobenzyl)amino]phenyl}-6-{[(2-chlorobenzyl)oxyjmethyl}pyrimidine-2,4-diamine;
5-{4-[(4-chlorobenzyl)amino]phenyl}-6-{[(4-chlorobenzyl)oxy]methyl}pyrimidine-2,4-diamine;
6-{[(2-bromobenzyl)oxy]methyl}-5-{4-[(4-chlorobenzyl)amino]phenyl}pyrimidine-2,4-diamine;
5-{4-[(4-chlorobenzyl)amino]phenyl}-6-({[3-(trifluoromethyl)benzyl]oxy}methyl)pyrimidine-2,4-diamine;
5-{4-[(4-chlorobenzyl)amino]phenyl}-6-({[4-(methylthio)benzyl]oxy}methyl)pyrimidine-2,4-diamine;
5-{4-[(4-chlorobenzyl)amino]phenyl}-6-{[(2,4-dimethylbenzyl)oxy]methyl}pyrimidine-2,4-diamine;
5-{4-[(4-chlorobenzyl)amino]phenyl}-6-{[(3,5-dimethylbenzyl)oxy]methyl}pyrimidine-2,4-diamine; t
5-{4-[(4-chlorobenzyl)amino]phenyl}-6-{[(2,3-dichlorobenzyl)oxy]methyl}pyrimidine-2,4-diamine;
5-{4-[(4-chlorobenzyl)amino]phenyl}-6-{[(2,5-dichlorobenzyl)oxy]methyl}pyrimidine-2,4-diamine;
5-{4-[(1,3-benzodioxol-4-ylmethyl)amino]phenyl}-6-[(benzyloxy)methyl]pyrimidine-2,4-diamine;
6-[(benzyloxy)methyl]-5-{4-[(2-furylmethyl)amino]phenyl}pyrimidine-2,4-diamine;
6-[(benzyloxy)methyl]-5-{4-[(tetrahydrofuran-3-ylmethyl)amino]phenyl}pyrimidine-2,4-diamine;
6-[(benzyloxy)methyl]-5-{4-[(pyridin-2-ylmethyl)amino]phenyl}pyrimidine-2,4-diamine;
6-[(benzyloxy)methyl]-5-{4-[(pyridin-3-ylmethyl)amino]phenyl}pyrimidine-2,4-diamine;
6-[(benzyloxy)methyl]-5-{4-[(pyridin-4-ylmethyl)amino]phenyl}pyrimidine-2,4-diamine;
6-[(benzyloxy)methyl]-5-{4-[(1H-imidazol-2-ylmethyl)amino]phenyl}pyrimidine-2,4-diamine;
6-[(benzyloxy)methyl]-5-{4-[(1H-imidazol-4-ylmethyl)amino]phenyl}pyrimidine-2,4-diamine;
6-[(benzyloxy)methyl]-5-[4-(dimethylamino)phenyl]pyrimidine-2,4-diaamine;
6-[(benzyloxy)methyl]-5-[4-(methylamino)phenyl]pyrimidine-2,4-diamine;
6-[(benzyloxy)methyl]-5-[4-(ethylamino)phenyl]pyrimidine-2,4-diamine;
6-[(benzyloxy)methyl]-5-[4-(propylamino)phenyl]pyrimidine-2,4-diamine;
6-[(benzyloxy)methyl]-5-[4-(isobutylamino)phenyl]pyrimidine-2,4-diamine;
6-[(benzyloxy)methyl]-5-[4-(neopentylamino)phenyl]pyrimidine-2,4-diamine;
6-[(benzyloxy)methyl]-5-{4-[(cyclopropylmethyl)amino]phenyl}pyrimidine-2,4-diamine;
5-{4-[(4-chlorobenzyl)amino]phenyl}-6-tetrahydrofuran-2-ylpyrimidine-2,4-diamine;
6-[(2-butoxyethoxy)methyl]-5-{4-[(4-chlorobenzyl)amino]phenyl}pyrimidine-2,4-diamine;
6-[(benzyloxy)methyl]-5-{4-[(1-ethylpropyl)amino]phenyl}pyrimidine-2,4-diamine;
4-{[(4-{2,4-diamino-6-[(benzyloxy)methyl]pyrimidin-5-yl}phenyl)amino]methyl}benzonitrile;
4-{[(4-{2,4-diamino-6-[(benzyloxy)methyl]pyrimidin-5-yl}phenyl)(methyl)amino]methyl}benzonitrile;
5-{4-[(4-chlorobenzyl)amino]phenyl}-6-[(3-methylbutoxy)methyl]pyrimidine-2,4-diamine;
5-(4-{[2-(benzyloxy)ethyl]amino}phenyl)-6-ethylpyrimidine-2,4-diamine;
6-[(benzyloxy)methyl]-5-(4-{[(6-chloropyridin-3-yl)methyl]amino}phenyl)pyrimidine-2,4-diamine;
N-benzyl-3-(2,6-diamino-5-{4-[(4-chlorobenzyl)amino]phenyl}pyrimidin-4-yl)propanamide;
3-(2,6-diamino-5-{4-[(4-chlorobenzyl)amino]pheny}pyrimidin-4-yl)-N-phenylpropanamide;
6-[(benzyloxy)methyl]-5-{4-[(1-pyridin-4-ylethyl)amino]phenyl}pyrimidine-2,4-diamine;
4-{1-[(4-{2,4-diamino-6-[(benzyloxy)methyl]pyrimidin-5-yl}phenyl)amino]ethyl}benzonitrile;
6-[(benzyloxy)methyl]-5-(4-{[1-(4-chlorophenyl)ethyl]amino}phenyl)pyrimidine-2,4-diamine;
6-[(benzyloxy)methyl]-5-{4-[(4-methoxybenzyl)amino]phenyl}pyrimidine-2,4-diamine;
6-[(benzyloxy)methyl]-5-{4-[(cyclohexylmethyl)amino]phenyl}pyrimidine-2,4-diamine;
3-(2,6-diamino-5-{4-[(4-chlorobenzyl)amino]phenyl}pyrimidin-4-yl)-N-(3-methylphenyl)propanamide;
6-[(benzyloxy)methyl]-5-{4-[(4-chlorobenzyl)oxy]phenyl}pyrimidine-2,4-diamine;
N-butyl-3-(2,6-diamino-5-{4-[(4-chlorobenzyl)amino]phenyl}pyrimidin-4-yl)propanamide;
6-[(benzyloxy)methyl]-5-(4-{[(4-chlorobenzyl)amino]methyl}phenyl)pyrimidine-2,4-diamine;
5-[4-(benzylamino)phenyl]-6-[(benzyloxy)methyl]pyrimidine-2,4-diamine;
6-[(benzyloxy)methyl]-5-(4-{[(4-nitrophenyl)amino]methyl}phenyl)pyrimidine-2,4-diamine;
4-{[(4-{2,4-diamino-6-[(cyclobutylmethoxy)methyl]pyrimidin-5-yl}phenyl)amino]methyl}benzonitrile;
tert-butyl 2-[(4-{2,4-Dianino-6-[(benzyloxy)methyl]pyrimidin-5-yl}phenyl)amino]ethylcarbamate;
4-[(4-{2,4-diamino-6-[(benzyloxy)methyl]pyrimidin-5-yl}phenoxy)methyl]benzonitrile;
5-{4-[(4-chlorobenzyl)amino]phenyl}-6-[(tetrahydro-2H-pyran-2-ylmethoxy)methyl]pyrimidine-2,4-diamine;
6-[(benzyloxy)methyl]-5-[4-({[6-(trifluoromethyl)pyridin-3-yl]methyl}amino)phenyl]pyrimidine-2,4-diamine;
4-[(4-{2,4-diamino-6-[(benzyloxy)methyl]pyrimidin-5-yl}benzyl)amino]benzonitrile;
3-[(4-{2,4-diamino-6-[(benzyloxy)methyl]pyrimidin-5-yl}phenoxy)methyl]benzonitrile;
5-{[(4-{2,4-diamino-6-[(benzyloxy)methyl]pyrimidin-5-yl}phenyl)amino]methyl}pyridine-2-carbonitrile;
6-[(benzyloxy)methyl]-5-{4-[2-(4-chlorophenyl)ethoxy]phenyl}pyrimidine-2,4-diamine;
6-[(benzyloxy)methyl]-5-[4-(pyridin-3-ylmethoxy)phenyl]pyrimidine-2,4-diamine;
6-[(benzyloxy)methyl]-5-{4-[(tetrahydro-2H-pyran-4-ylmethyl)amino]phenyl}pyrimidine-2,4-diamine;
6-[(benzyloxy)methyl]-5-(4-{[4-(trifluoromethoxy)benzyl]amino}phenyl)pyrimidine-2,4-diamine;
4-{[(4-{2,4-diamino-6-[(benzyloxy)methyl]pyrimidin-5-yl}phenyl)amino]methyl}pyridine-2-carbonitrile;
6-[(4-{2,4-diamino-6-[(benzyloxy)methyl]pyrimidin-5-yl}benzyl)amino]nicotinonitrile;
5-{4-[(4-chlorobenzyl)amino]phenyl}-6-{[(3-chlorobenzyl)oxy]methyl}pyrimidine-2,4-diamine;
5-{4-[(4-chlorobenzyl)amino]phenyl}-6-{[(2-methylbenzyl)oxy]methyl}pyrimidine-2,4-diamine;
5-{4-[(4-chlorobenzyl)amino]phenyl}-6-[(cyclohexylmethoxy)methyl]pyrimidine-2,4-diamine;
6-[(benzyloxy)methyl]-5-{4-[(4-nitrobenzyl)aniino]phenyl}pyriridine-2,4-diamine;
6-ethyl-5-{4-[(4-nitrobenzyl)amino]phenyl}pyrimidine-2,4-diamine;
6-[(benzyloxy)methyl]-5-(4-{[(2-chloropyridin-4-yl)methyl]amino}phenyl)pyrimidine-2,4-diamine;
6-[(benzyloxy)methyl]-5-{4-[(pyrimidin-5-ylmethyl)amino]phenyl}pyrimidine-2,4-diamine;
6-[(benzyloxy)methyl]-5-{4-[(thien-2-ylmethyl)amino]phenyl}pyrimidine-2,4-diamine;
6-[(benzyloxy)methyl]-5-{4-[(thien-3-ylmethyl)amino]phenyl}pyrimidine-2,4-diamine;
6-[(benzyloxy)methyl]-5-[4-({[1-(4-chlorophenyl)ethyl]amino}methyl)phenyl]pyrimidine-2,4-diamine;
6-[(benzyloxy)methyl]-5-(4-{[2-(4-nitrophenyl)ethyl]amino}phenyl)pyrimidine-2,4-diamine;
6-[(benzyloxy)methyl]-5-(4-{[2-(4-chlorophenyl)ethyl]amino}phenyl)pyrimidine-2,4-diamine;
6-[(benzyloxy)methyl]-5-{4-[(cycloheptylamino)methyl]phenyl}pyrimidine-2,4-diamine;
6-[(benzyloxy)methyl]-5-[4-(pyridin-4-ylmethoxy)phenyl]pyrimidine-2,4-diamine;
6-{[(3-Methylbut-2-enyl)oxy]methyl}-5-(4-{[4-(methylsulfonyl)benzyl]amino}phenyl)pyrimidine-2,4-diamine;
3-[2,6-diamino-5-(4-{[4-(methylsulfonyl)benzyl]amino}phenyl)pyrimidin-4-yl]-N-(3,5-dimethylbenzyl)propanamide;
6-[(benzyloxy)methyl]-5-(4-{[3-fluoro-4-(methylsulfonyl)benzyl]amino}phenyl)pyrimidine-2,4-diamine;
5-(3-amino-4-{[4-(methylsulfonyl)benzyl]amino}phenyl)-6-ethylpyrimidine-2,4-diamine;
1-[4-({[4-(2,4-diamino-6-{[(3-methylbut-2-enyl)oxy]methyl}pyrimidin-5-yl)phenyl]amino}methyl)phenyl]ethanone;
6-[(3-methylbutoxy)methyl]-5-(4-{[4-(methylsulfonyl)benzyl]amino}phenyl)pyrimidine-2,4-diamine;
6-[(benzyloxy)methyl]-5-(4-{[4-(methylsulfonyl)benzyl]amino}phenyl)pyrimidine-2,4-diamine;
5-(3-chloro-4-{[4-(methylsulfonyl)benzyl]amino}phenyl)-6-ethylpyrimidine-2,4-diamine;
6-[(benzyloxy)methyl]-5-(4-{[4-(methylsulfonyl)benzyl]oxy}phenyl)pyrimidine-2,4-diamine;
6-{[methyl(phenyl)amino]methyl}-5-(4-{[4-(methylsulfonyl)benzyl]amino}phenyl)pyrimidine-2,4-diamine;
6-ethyl-5-(4-{methyl[4-(methylsulfonyl)benzyl]amino}phenyl)pyrimidine-2,4-diamine;
6-(anilinomethyl)-5-(4-{[4-(methylsulfonyl)benzyl]amino}phenyl)pyrimidine-2,4-diamine;
6-[(benzyloxy)methyl]-5-(4-{[4-(ethylsulfonyl)benzyl]amino}phenyl)pyrimidine-2,4-diamine;
6-[(2-furylmethoxy)methyl]-5-(4-{[4-(methylsulfonyl)benzyl]amino}phenyl)pyrimidine-2,4-diamine;
5-(4-{[4-(methylsulfonyl)benzyl]amino}phenyl)-6-neopentylpyrimidine-2,4-diamine;
1-[4-({[4-(2,4-diamino-6-isopropylpyrimidin-5-yl)phenyl]amino}methyl)phenyl]ethanol;
6-ethyl-5-{1-[4-(methylsulfonyl)benzyl]-1H-benzimidazol-5-yl}pyrimidine-2,4-diamine;
5-(4-{[3-chloro-4-(methylsulfonyl)benzyl]amino}phenyl)-6-ethylpyrimidine-2,4-diamine;
6-isopropyl-5-(4-{[4-(methylsulfonyl)benzyl]amino}phenyl)pyrimidine-2,4-diamine;
1-(4-{[(4-{2,4-diamino-6-[(benzyloxy)methyl]pyrimidin-5-yl}phenyl)amino]methyl}phenyl)propan-1-one;
6-{[(2,3-dimethoxyphenyl)amino]methyl}-5-(4-{[4-(methylsulfonyl)benzyl]amino}phenyl)pyrimidine-2,4-diamine;
6-ethyl-5-(4-{[3-methyl-4-(methylsulfonyl)benzyl]amino}phenyl)pyrimidine-2,4-diamine;
5-(4-{[3,5-difluoro-4-(methylsulfonyl)benzyl]aamino}phenyl)-6-ethylpyrimidine-2,4-diamine;
6-[(benzyloxy)methyl]-5-[4-({4-[(trifluoromethyl)sulfonyl]benzyl}amino)phenyl]pyrimidine-2,4-diamine;
6-ethyl-5-{1-[4-(methylsulfonyl)benzyl]-1H-indol-5-yl}pyrimidine-2,4-diamine;
6-ethyl-5-(4-{[4-(methylsulfonyl)benzyl]amino}phenyl)pyrimidine-2,4-diamine;
methyl 4-{[(4-{2,4-diamino-6-[(benzyloxy)methyl]pyrimidin-5-yl}phenyl)amino]methyl}benzoate;
6-[(benzylamino)methyl]-5-(4-{[4-(methylsulfonyl)benzyl]amino}phenyl)pyrimidine-2,4-diamine;
1-(4-{[4-(2,4-diamino-6-ethylpyrimidin-5-yl)benzyl]amino}phenyl)ethanone;
6-(methoxymethyl)-5-(4-{[4-(methylsulfonyl)benzyl]amino}phenyl)pyrimidine-2,4-diamine;
6-ethyl-5-(4-{[(4-nitrophenyl)amino]methyl}phenyl)pyrimidine-2,4-diamine;
6-[(benzyloxy)methyl]-5-(4-{[4-(1,3-oxazol-2-yl)benzyl]amino}phenyl)pyrimidine-2,4-diamine;
6-hexyl-5-(4-{[4-(methylsulfonyl)benzyl]amino}phenyl)pyrimidine-2,4-diamine;
6-But-3-enyl-5-(4-{[4-(methylsulfonyl)benzyl]amino}phenyl)pyrimidine-2,4-diamine;
6-ethyl-5-[4-({[4-(methylsulfonyl)phenyl]amino}methyl)phenyl]pyrimidine-2,4-diamine;
6-(2,3-dihydro-1H-indol-1-ylmethyl)-5-(4-{[4-(methylsulfonyl)benzyl]amino}phenyl)pyrimidine-2,4-diamine;
6-(3,4-dihydroisoquinolin-2(1H)-ylmethyl)-5-(4-{[4-(methylsulfonyl)benzyl]amino}phenyl)pyrimidine-2,4-diamine;
5-(4-{[4-(methylsulfonyl)benzyl]aamino}phenyl)-6-{[(thien-2-ylmethyl)amino]methyl}pyrimidine-2,4-diamine;
6-bicyclo[2.2.1]hept-2-yl-5-(4-{[4-(methylsulfonyl)benzyl]amino}phenyl)pyrimidine-2,4-diamine;
N-4-benzyl-5-(4-{[4-(methylsulfonyl)benzyl]amino}phenyl)pyrimidine-2,4,6-triamine;
6-cyclobutyl-5-(4-{[4-(methylsulfonyl)benzyl]amino}phenyl)pyrimidine-2,4-diamine;
6-cyclopropyl-5-(4-{[4-(methylsulfonyl)benzyl] amino}phenyl)pyrimidine-2,4-diamine;
6-(2,3-dihydro-1,4-benzodioxin-2-yl)-5-(4-{[4-(methylsulfonyl)benzyl]amino}phenyl)pyrimidine-2,4-diamine;
6-[(1R)-1-(benzyloxy)ethyl]-5-(4-{[4-(methylsulfonyl)benzyl]amino}phenyl)pyrimidine-2,4-diamine;
6-tert-butyl-5-(4-{[4-(methylsulfonyl)benzyl]amino}phenyl)pyrimidine-2,4-diamine;
6-cyclopentyl-5-(4-{[4-(methylsulfonyl)benzyl]amino}phenyl)pyrimidine-2,4-diamine;
6-cyclohexyl-5-(4-{[4-(methylsulfonyl)benzyl]amino}phenyl)pyrimidine-2,4-diamine;
6-(2,3-dihydro-1-benzofuran-2-yl)-5-(4-{[4-(methylsulfonyl)benzyl]amino}phenyl)pyrimidine-2,4-diamine;
6-(cyclopentylmethyl)-5-(4-{[4-(methylsulfonyl)benzyl]amino}phenyl)pyrimidine-2,4-diamine;
5-(4-{[4-(methylsulfonyl)benzyl]amino}phenyl)-6-tetrahydro-2H-pyran-4-ylpyrimidine-2,4-diamine;
6-ethyl-5-{3-ethyl-1-[4-(methylsulfonyl)benzyl]-1H-indol-5-yl}pyrimidine-2,4-diamine;
5-(4-{[4-(methylsulfonyl)benzyl]amino}phenyl)-6-[(2S)-tetrahydrofuran-2-yl]pyrimidine-2,4-diamine;
5-(4-{[4-(methylsulfonyl)benzyl]amino}phenyl)-6-[(2R)-tetrahydrofuran-2-yl]pyrimidine-2,4-diamine;
6-ethyl-5-{2-methyl-1-[4-(methylsulfonyl)benzyl]-1H-benzimidazol-5-yl}pyrimidine-2,4-diamine;
5-(4-{[4-(methylsulfonyl)benzyl]amino}phenyl)-N-4-(2-phenylethyl)pyrimidine-2,4,6-triamine;
5-{2-cyclopentyl-1-[4-(methylsulfonyl)benzyl]-1H-benzimidazol-5-yl}-6-ethylpyrimidine-2,4-diamine;
6-ethyl-5-{1-[4-(methylsulfonyl)benzyl]-2-phenyl-1H-benzimidazol-5-yl}pyrimidine-2,4-diamine; and
6-ethyl-5-(3-methyl-4-{[4-(methylsulfonyl)benzyl]amino}phenyl)pyrimidine-2,4-diamine.

12. A pharmaceutical composition comprising a therapeutically effective amount of a compound of formula (I) and a pharmaceutically suitable carrier.

13. A method of treating a disorder regulated by GHS-Rs in a mammal, comprising administring of a compound of formula (I).

14. A method of treating disorders regulated by the activation of GHS-R, including Prader-Willi syndrome, eating disorder, weight gain, weight-loss maintainance following diet and exercise, obesity, and disorders associated with obesity such as non-insulin dependent diabetes mellitus in a mammal comprising administrating a compound of formula (I).

15. A pharmaceutical composition comprising a therapeutically effective amount of a compound of formula (I) coadminstered with an effective amount of a folate supplement.
